(19) United States
(12) Patent Application Publication
(10) Pub. No.: US 2026/0055144 A1
(43) Pub. Date: Feb. 26, 2026

(54) RECOMBINANT ADENO-ASSOCIATED VIRUSES AND USES THEREOF

(71) Applicant: REGENXBIO INC., Rockville, MD (US)

(72) Inventors: Elad Firnberg, Rockville, MD (US); Andrew Mercer, Rockville, MD (US); April R. Tepe, Boston, MA (US); Samantha Yost, Rockville, MD (US); Ye Liu, Rockville, MD (US)

(21) Appl. No.: 19/105,376

(22) PCT Filed: Aug. 24, 2023
(86) PCT No.: PCT/US2023/072875

§ 371 Date: Feb. 21, 2025

Related U.S. Application Data
(60) Provisional application No. 63/400,724, filed on Aug. 24, 2022.

(51) Int. Cl. C07K 14/005; C12N 15/86

(52) U.S. Cl. CPC C07K 14/005; C12N 15/86; C12N 2750/14122; C12N 2750/14143; C12N 2750/14151

(57) ABSTRACT

The present invention relates to recombinant adeno-associated viruses (rAAVs) having capsid proteins engineered to include amino acid sequences that confer and/or enhance desired properties. In particular, the invention provides engineered capsid proteins comprising peptide insertions from heterologous proteins inserted within or near variable region IV (VR-IV) of the virus capsid, such that the insertion is surface exposed on the AAV particle. The invention also provides capsid proteins that direct rAAVs to target tissues, in particular, capsid proteins comprising peptides that are inserted into surface-exposed variable regions using a method for replacing a stop codon in the capsid gene with such peptide insert. The invention provides such methods for making the rAAV vectors into vector libraries and selecting the rAAV vectors having peptide inserts that target the rAAV to particular tissues of interest, and thus deliver therapeutics for treating disorders.

### SEQUENCE LISTING

The Sequence Listing submitted herewith as an XML file named 38013_0030P1_SL.xml, created on Aug. 24, 2023, and having a size of 530,415 bytes is hereby incorporated by reference pursuant to 37 C.F.R. §§ 1.831-1.835.

### 1. FIELD OF THE INVENTION

The present invention relates to recombinant adeno-associated viruses (rAAVs) having capsid proteins engineered to include amino acid sequences that confer and/or enhance desired properties when incorporated into an rAAV capsid. In particular, the invention provides engineered capsid proteins comprising peptide insertions inserted within or near variable region IV (VR-IV) of the virus capsid, such that the insertion is surface exposed on the AAV particle. The invention also provides capsid proteins that direct rAAVs to target tissues, in particular, capsid proteins derived from rAAV libraries, and provides such libraries constructed to reduce the parental vector production and thus overrepresentation of the parental capsid in the library and comprising random peptides inserted into surface-exposed variable regions to target rAAVs to and/or improve transduction of tissues of interest, including the muscle tissue, and deliver therapeutics for treating muscle disorders.

### 2. BACKGROUND

The use of recombinant adeno-associated viruses (AAV) as gene delivery vectors is a promising avenue for the treatment of many patients with unmet needs and/or rare disease. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Our current understanding of these capsids, their utility and function has allowed for efforts to further hone the efficiency and effectiveness of carrying therapeutic genome DNA, such as directing tissue tropism to deliver such DNA into target cells, while reducing the tropism to tissues where vector transduction and/or expression of transgene is undesirable, in order to safely ameliorate serious diseases.

Due to low pathogenicity and the promise of long-term, targeted gene expression, recombinant AAVs (rAAVs) have been used as gene transfer vectors, in which therapeutic sequences are packaged into capsids. Such vectors have been used to deliver a variety of therapeutic genes, thus many gene therapy products are currently in clinical development. Recombinant AAVs, such as AAV9, have demonstrated desirable muscle and neurotropic properties and clinical trials using recombinant AAV9 for treatment of muscle diseases, such as dystrophinopathies, are underway. However, attempts to identify rAAV capsids with desirable properties in human subjects are limited by the methods that select for them.

There remains a need for rAAV vectors with enhanced tropism to particular tissues and properties for use, e.g., in high transduction to muscles to delivery therapies in treating disorders such as dystrophinopathies. There also is a need for improved methods for identifying such rAAV vectors with enhanced tissue-specific targeting and/or enhanced tissue-specific transduction to deliver therapies using lower dosing than is currently available.

### 3. SUMMARY OF THE INVENTION

Provided are recombinant adeno-associated viruses (rAAVs) having capsid proteins engineered to include amino acid sequences that confer and/or enhance desired properties such as tissue targeting, transduction or expression of the rAAV genome. In particular, provided are engineered capsid proteins comprising peptide insertions, derived from peptide libraries, inserted within or near variable region IV (VR-IV) of the virus capsid, or, in certain embodiments, within or near variable region VIII (VR-VIII), such that the peptide insertion is surface exposed on the AAV particle when the engineered capsid protein is incorporated into an rAAV particle. In embodiments, the peptide is 4 to 7 amino acids of one of the peptides having an amino acid sequence of SEQ ID NO: 1-138 (Tables 4, 5, 14, 15 or 16). In embodiments, the insertion is immediately after an amino acid residue corresponding to one of the amino acids 451 to 461 of the AAV9 capsid protein (SEQ ID NO: 151 and as numbered, e.g., in FIG. 1), including after the amino acid 454 (i.e., between amino acid 454 and 455) of the AAV9 capsid or in the AAV9.AAA (e.g., SEQ ID NO: 158) capsid protein; or in a capsid protein of a different AAV type after a residue that corresponds to the amino acid 454 of AAV9, see alignment in FIG. 1 or for AAV types not included in FIG. 1, a similar amino acid sequence alignment of the AAV9 capsid protein sequence (SEQ ID NO: 151), such as 451-461 of AAV9 is the same as AAVhu.32 capsid protein sequence (SEQ ID NO: 148) as aligned in FIG. 1; and the AAV capsid protein as would be well known in the art). The capsid protein may be an AAV9 capsid protein but may also be any AAV capsid protein, such as AAV type 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype hu.31 (AAVhu.31), serotype hu.32 (AAVhu.32), serotype rh10 (AAVrh10), serotype rh20 (AAVrh20), serotype hu.37 (AVVhu.37), serotype rh39 (AAVrh39), and serotype rh74 (AAVrh74), or a variant AAV9 capsid protein AAV9.AAA (496NNN/AAA498 amino acid substitutions), (see, e.g., alignments presented in FIG. 1) or may be a capsid protein which has 90%, 95% or 99% amino acid sequence identity to one of the foregoing capsid proteins. Thus, provided is an engineered capsid protein comprising a peptide insertion from a heterologous protein (i.e., not an AAV capsid protein) inserted immediately after or near an amino acid corresponding to the amino acid residue at position 454 of AAV9, as numbered in FIG. 1.

Also provided are engineered capsid proteins that direct rAAVs to target tissues, in particular, capsid proteins comprising peptides (derived from peptide libraries) or a peptide that promotes tissue targeting and/or cellular uptake and/or expression of the rAAV genome, that are inserted into surface-exposed variable regions and that target rAAVs to muscle tissue (skeletal muscle and/or heart), including to the central nervous system, and neurons within the CNS, and to retinal tissue, and deliver therapeutics for treating neurological and ocular disorders. These peptides, including 4, 5, 6, or all 7 contiguous amino acids of one of the peptides in Tables 4, 5, 14, 15, or 16 (SEQ ID Nos: 1-138) are advantageously inserted into the amino acid sequence of the capsid protein such that, when the capsid protein is incorporated into the AAV particle, the inserted peptide is surface exposed. These peptides are inserted immediately after one of the amino acid residues of, or after one of the amino acids corresponding to the amino acid, 585-593 of AAV9 capsid (SEQ ID NO: 151) or AAV9.AAA capsid (SEQ ID NO: 158, e.g. see FIG. 1 for alignment), or immediately after one of the amino acid residues of, or after one of the amino acids corresponding to 451-461 of the AAV9 capsid or AAV9.AAA capsid and amino acids corresponding to any one of positions 451-461 of the AAV9 capsid (SEQ ID NO: 151, see, e.g. FIG. 1 for alignment) or AAV9.AAA capsid. Exemplary modified capsid sequences are provided in Table 17, including capsid proteins having amino acid sequence of one of SEQ ID Nos: 159 to 266 (AAV9.AAA parental capsid) and 268 to 375 (AAVhu.32.AAA parental capsid). In other embodiments, a 4, 5, 6, or 7 amino acid portion of a peptide having an amino acid sequence of SEQ ID NO; 113, 114 or 115 is inserted immediately after position 577 or 578 of AAV5 (SEQ ID NO: 143). In embodiments, provided are capsid proteins and capsid incorporating capsid proteins having an amino acid sequence of SEQ ID NO; 376, 377 or 378 (AAV5 parental capsid).

Provided are engineered capsid proteins comprising peptides that target specific tissues, including, when incorporated into an rAAV vector as a capsid to promote or increase cellular uptake and/or integration of an rAAV genome and/or expression of a transgene within the rAAV genome wherein the peptides are inserted into surface-exposed variable regions of the capsid protein. In certain embodiments, the peptides target and/or promote transduction or genome integration in cells of muscle, for example, at least 4 contiguous amino acids or at least 5, 6, or 7 contiguous amino acids of any of the peptides in Tables 4, 5, 14, 15 or 16, and capsids containing one of these peptides, for example, immediately after one of the positions 451-461, including after position 454, of AAV9, AAV9.AAA, AAVhu.32, or AAVhu.32.AAA, preferentially target the rAAV with the capsid to muscle tissue, and, in embodiments detargeting the liver. In embodiments, the capsid having the peptide insert targets and transduces neurons within the CNS, including relative to the transduction of astrocytes in the CNS. In other embodiments, the inserted peptide is at least 4 contiguous amino acids, and in addition the capsid is engineered to have one or more amino acid substitutions which may improve tropism, transduction or reduce immune neutralizing activity. Such amino acid modifications include amino acid substitutions of the NNN (asparagines) at 496 to 498 with AAA (alanines) in the AAV9 capsid. Other parental capsids having the peptide insertion may also have substitutions of the NNN (asparagines) at 498 to 500 with AAA (alanines) in the AAV8 capsid or of the NNN (asparagines) at 497 to 499 with AAA (alanines) in the AAVhu.32 capsid or corresponding substitutions in other AAV type capsids. Still other parental capsids having the peptide insertion may also have substitutions of S263F/S269T/A273T in AAV9, and corresponding substitutions in other AAV type capsids, W530R or Q474A in AAV9, and corresponding substitutions in other AAV type capsids, and/or A269S in AAV8, and corresponding substitutions in other AAV type capsids.

Also provided are engineered capsid proteins that promote transduction of the rAAV in one or more tissues, including one or more cell types, upon systemic, intravenous, intraperitoneal, or intramuscular administration, wherein the capsid proteins comprise a peptide of Tables 4, 5, 14, 15 or 16 (SEQ ID Nos: 1 to 138) that is inserted into a surface-exposed variable region (VR) of the capsid, e.g. VR-I, VR-IV or VR-VIII, or after the first amino acid of VP2, e.g., immediately after residue 138 of the AAV9 capsid (amino acid sequence of SEQ ID NO: 151) or immediately after the corresponding residue of another AAV capsid, or alternatively is engineered with one or more of the amino acid substitutions described herein, and transduction of the AAV having the engineered capsid in the at least one tissue (such as muscle (skeletal and/or heart, retina, or CNS, including CNS neurons) is increased upon said administration compared (for example, 1 fold, 2 fold, 5 fold, 10 fold or 20 fold greater) to the transduction of the AAV having the corresponding unengineered capsid (parental capsid) or a reference capsid such as AAV9, AAV9.AAA, AAVhu.32 or AAVhu.32.AAA. Such capsids may also exhibit reduced transduction of one or more tissues, including liver, heart or astrocytes upon administration compared (for example, 1 fold, 2 fold, 5 fold, 10 fold or 20 fold greater) to the transduction of the AAV in tissues such as muscle, retina or CNS or having the corresponding unengineered capsid (parental capsid) or a reference capsid such as AAV9, AAV9.AAA, AAVhu.32 or AAVhu.32.AAA In certain embodiments, transduction is measured by detection of transgene, such as DNA, RNA transcript or expressed protein in the cell, e.g. reporter transgenes may be utilized and measured such as GFP fluorescence.

In certain embodiments, provided are rAAVs incorporating the engineered capsids described herein, including rAAVs with genomes comprising a transgene of therapeutic interest. Plasmids and cells for production of a pool (stock) of plasmids for the production of the rAAVs are described herein. Packaging cells and methods for the production of the rAAVs comprising the engineered capsids are also provided herein. Method of treatment by delivery of, and pharmaceutical compositions comprising, the engineered rAAVs described herein are provided. Also provided are methods of manufacturing the rAAVs with the engineered capsids described herein.

Also provided are methods of making the capsid libraries having the peptide inserts. In embodiments, the method comprises providing a starting plasmid that contains a gene expression cassette encoding a capsid gene, wherein a stop codon is placed at a target insertion site within the capsid gene, randomizing a repertoire of nucleic acids encoding randomized peptides to produce a peptide library; creating individual plasmids based on the starting plasmid 1) each having a nucleic acid encoding a random peptide from the peptide library inserted at the target insertion site of the capsid gene thus replacing the stop codon, and 2) each encoding a barcode for identification of the capsid gene having the insert placed before the 5′- or after the 3′-end of the capsid gene; collecting the individual plasmids to form a population or collection of plasmids encoding the capsids with peptide inserts and transfecting with the collection of plasmids with plasmids encoding a recombinant rAAV genome containing a transgene, including one that is detectable, and necessary genes to produce a collection of rAAV vectors encapsidating the rAAV genome containing the transgene, wherein the rAAV vectors have capsids with the encoded capsid protein containing a library peptide insert. In embodiments, the parental AAV is AAV9, AAV9.AAA, AAVhu.32, AAVhu.32.AAA, or AAV5 or any other suitable AAV serotype, for example, as in Table 3. The insertion site may be in VR-IV, including immediately after one of amino acids 451-461 of AAV9 or corresponding to one of those residues or in VR-VIII, including immediately after amino acid 588 of AAV9 or corresponding to that position in a different AAV capsid type (see FIG. 1 for alignment).

The library of modified capsids is harvested from these cells. In embodiments, the rAAV library population produced has high levels of capsids having the peptide inserts, including 85%, 90%, 95% or 98%, 99% or even 100%.

The invention is illustrated by way of examples infra describing the construction of engineered rAAV9 capsids having peptide inserts designed from rAAV libraries enabling the detection of desirable properties such as tissue targeting.

### 3.1. Embodiments

1. A recombinant adeno-associated virus (rAAV) capsid protein comprising a peptide insertion of at least 4 and up to 7 contiguous amino acids, said peptide insertion being immediately after an amino acid residue corresponding to one of amino acids 451 to 461 of AAV9 capsid protein of FIG. 1, wherein said peptide insertion has an amino acid sequence of one of SEQ ID NOs: 1-138,
   and wherein said capsid protein is a wild-type capsid protein or a variant capsid protein having up to three amino acid substitutions, and
   wherein an rAAV vector prepared from the capsid protein comprising the peptide insertion has enhanced tropism to a target tissue compared to an rAAV vector prepared from a capsid protein without the peptide insertion or a reference capsid protein.
2. The rAAV capsid protein of embodiment 1, wherein said capsid protein is from at least one AAV type selected from AAV type 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype hu.31 (AAVhu.31), serotype hu.32 (AAVhu.32), serotype rh10 (AAVrh10), serotype rh20 (AAVrh20), serotype hu.37 (AVVhu.37), serotype rh39 (AAVrh39), and serotype rh74 (AAVrh74), or said variant capsid protein is AAV9.AAA or AAVhu.32.AAA or a capsid protein that has 90%, 95%, or 99% sequence identity thereto.
3. The rAAV capsid protein of embodiment 1 or 2, wherein said peptide insertion occurs immediately after one of the amino acid residues within:
   (a) 450-459 of AAV1 capsid amino acid sequence (SEQ ID NO: 139);
   (b) 449-458 of AAV2 capsid amino acid sequence (SEQ ID NO: 140);
   (c) 449-459 of AAV3 capsid amino acid sequence (SEQ ID NO: 141);
   (d) 443-453 of AAV4 capsid amino acid sequence (SEQ ID NO: 142);
   (e) 442-445 of AAV5 capsid amino acid sequence (SEQ ID NO: 143);
   (f) 450-459 of AAV6 capsid amino acid sequence (SEQ ID NO: 144);
   (g) 451-461 of AAV7 capsid amino acid sequence (SEQ ID NO: 145);
   (h) 451-461 of AAV8 capsid amino acid sequence (SEQ ID NO: 146);
   (i) 451-461 of AAV9 capsid amino acid sequence (SEQ ID NO: 151);
   (j) 451-461 of AAVhu.32 capsid amino acid sequence (SEQ ID NO: 148);
   (k) 452-461 of AAVrh10 capsid amino acid sequence (SEQ ID NO: 152);
   (l) 452-461 of AAVrh20 capsid amino acid sequence (SEQ ID NO: 155);
   (m) 452-461 of AAVhu.37 capsid amino acid sequence (SEQ ID NO: 153);
   (n) 452-461 of AAVrh39 capsid amino acid sequence (SEQ ID NO: 150);
   (o) 452-461 of AAVrh74 capsid amino acid sequence (SEQ ID NO: 156 or SEQ ID NO: 157); or
   (p) 452-461 of AAV9.AAA capsid amino acid sequence (SEQ ID NO: 158) as depicted in FIG. 1.
4. The rAAV capsid protein of embodiment 3, wherein said peptide insertion occurs after an amino acid residue corresponding to one of amino acids I451, N452, G453, S454, G455, Q456, N457, Q458, Q459, T460, or L461 of the AAV9 capsid or variant AAV9.AAA capsid.
5. The rAAV capsid protein of embodiment 4, wherein said peptide insertion occurs after an amino acid residue corresponding to amino acid S454 of the AAV9 capsid protein, the AAV.32 capsid protein, variant AAV9.AAA capsid protein, or variant AAVhu.32.AAA capsid protein.
6. The rAAV capsid protein of any one of embodiments 1-5 wherein the peptide is 7 amino acids.
7. The rAAV capsid protein of any one of embodiments 1-6 wherein the reference capsid protein is AAV9, AAV9.AAA, AAVhu32, or AAVhu32.AAA.
8. The rAAV capsid protein of any one of embodiments 1 through 7, wherein said peptide insertion comprises an amino acid sequence of at least 4 and up to 7 contiguous amino acids of SEQ ID NO: 135, SEQ ID NO: 136, SEQ ID NO: 137 or SEQ ID NO: 138, where SEQ ID NO: 135 is X₁-Q-V-X₂-X₃-X₄-X₅, wherein X₁ is V or A, X₂ is S, G, V or A, X₃ is R or H, X₄ is any amino acid and X₅ is S, G, V or A, where SEQ ID NO: 136 is X₁-Q-V-X₂-X₃-X₄-X₅, wherein X₁ is V or A, X₂ is S, G, V or A, X₃ is R or H, X₄ is any amino acid and X₅ is S or A, where SEQ ID NO: 137 is X₁-Q-V-X₂-X₃-X₄-X₅, wherein X₁ is V or A, X₂ is S, G, V or A, X₃ is R or H, X₄ is T, S, V, Y, A or P and X₅ is S, G, V or A, or where SEQ ID NO: 138 is X₁-Q-V-X₂-X₃-X₄-X₅, wherein X₁ is V or A, X₂ is S, G, V or A, X₃ is R or H, X₄ is T, S, V, Y, A or P and X₅ is S or A.
9. The rAAV capsid protein of embodiment 8, wherein the peptide insertion has an amino acid sequence of VQVGRAA (SEQ ID NO: 5), VQVGRTS (SEQ ID NO: 8), AQVGRAS (SEQ ID NO: 24), VQVGRVS (SEQ ID NO: 36), VQVGRSS (SEQ ID NO: 44), VQVGRYS (SEQ ID NO: 130), VQVGRAS (SEQ ID NO: 131), VQVGRPS (SEQ ID NO: 132), VQVVRPS (SEQ ID NO: 133) or VQVGHAS (SEQ ID NO: 134).
10. The rAAV capsid protein of embodiment 8 or 9 which has an amino acid sequence of SEQ ID No. 263, 264, 265 or 266.
11. The rAAV capsid protein of any one of embodiments 1-7, wherein said peptide insertion comprises an amino acid sequence of at least 4 and up to 7 contiguous amino acids of SGTVIRS (SEQ ID NO: 1), TRQYVPG (SEQ ID NO: 4), VQVGRTS (SEQ ID NO: 8), RGAVQKV (SEQ ID NO: 13), GQVHQAR (SEQ ID NO: 32), TSGGQIR (SEQ ID NO: 38), HMGHSGK (SEQ ID NO: 88), MRAVSQL (SEQ ID NO: 109), PRQYVPG (SEQ ID NO: 110), RSSSGR (SEQ ID NO: 111), VVKSTKS (SEQ ID NO: 112), RHVSASD (SEQ ID NO: 113), VRSDRDQ (SEQ ID NO: 114), or TVVTSIN (SEQ ID NO: 115).
12. The rAAV capsid protein of embodiment 11 wherein said peptide insertion is SGTVIRS (SEQ ID NO: 1), TRQYVPG (SEQ ID NO: 4), VQVGRTS (SEQ ID NO: 8), RGAVQKV (SEQ ID NO: 13), GQVHQAR (SEQ ID NO: 32), TSGGQIR (SEQ ID NO: 38), HMGHSGK (SEQ ID NO: 88), MRAVSQL (SEQ ID NO: 109), PRQYVPG (SEQ ID NO: 110), RSSSGR (SEQ ID NO: 111), VVKSTKS (SEQ ID NO: 112), RHVSASD (SEQ ID NO: 113), VRSDRDQ (SEQ ID NO: 114), or TVVTSIN (SEQ ID NO: 115).
13. The rAAV capsid protein of any one of embodiments 1 through 7, wherein said peptide insertion comprises an amino acid sequence of at least 4 and up to 7 contiguous amino acids of AQVGRAS (SEQ ID NO: 24), SVTSVRV (SEQ ID NO: 37), SIAKNSA (SEQ ID NO: 76), DGRRIGV (SEQ ID NO: 116), TSGERRG (SEQ ID NO: 117), PSSVQHR (SEQ ID NO: 118), SSSVQHR (SEQ ID NO: 119), SGMQERR (SEQ ID NO: 120), LERGNLE (SEQ ID NO: 121), YRDVRQT (SEQ ID NO: 122), PSAVQHR (SEQ ID NO: 123), YVGGRAV (SEQ ID NO: 124), IGSRGVA (SEQ ID NO: 125), SDVSRPR (SEQ ID NO: 126), GSVRQAA (SEQ ID NO: 127), QSPHTSQ (SEQ ID NO: 128), or ASQAYHG (SEQ ID NO: 128).
14. The rAAV capsid protein of embodiment 13, wherein said peptide insertion is AQVGRAS (SEQ ID NO: 24), SVTSVRV (SEQ ID NO: 37), SIAKNSA (SEQ ID NO: 76), DGRRIGV (SEQ ID NO: 116), TSGERRG (SEQ ID NO: 117), PSSVQHR (SEQ ID NO: 118), SSSVQHR (SEQ ID NO: 119), SGMQERR (SEQ ID NO: 120), LERGNLE (SEQ ID NO: 121), YRDVRQT (SEQ ID NO: 122), PSAVQHR (SEQ ID NO: 123), YVGGRAV (SEQ ID NO: 124), IGSRGVA (SEQ ID NO: 125), SDVSRPR (SEQ ID NO: 126), GSVRQAA (SEQ ID NO: 127), QSPHTSQ (SEQ ID NO: 128), or ASQAYHG (SEQ ID NO: 128).
15. The rAAV capsid protein of embodiment 14, wherein said peptide insertion is AQVGRAS (SEQ ID NO: 24), PSSVQHR (SEQ ID NO: 118), YVGGRAV (SEQ ID NO: 124), IGSRGVA (SEQ ID NO: 125) or GSVRQAA (SEQ ID NO: 127).
16. The rAAV capsid protein of embodiment 14, wherein said peptide insertion is DGRRIGV (SEQ ID NO: 116), SSSVQHR (SEQ ID NO: 119), YRDVRQT (SEQ ID NO: 122) or PSAVQHR (SEQ ID NO: 123).
17. The rAAV capsid protein of embodiment 1, having the amino acid sequence of any one of the amino acid sequences of SEQ ID NO: 159-266 and 268-375.
18. The rAAV capsid protein of any one of the preceding embodiments, wherein said target tissue with enhanced tropism is skeletal muscle or heart muscle.
19. The rAAV capsid protein of embodiment 18, wherein an rAAV vector prepared from the capsid protein exhibits an at least 2 fold, 5 fold, 10 fold, 15 fold, 20 fold, or 25 fold greater transduction of skeletal muscle and/or heart muscle than an rAAV vector prepared from the capsid protein without the peptide insert or the reference capsid protein.
20. The rAAV capsid protein of any one of the preceding embodiments wherein the target tissue is CNS neurons.
21. The rAAV capsid protein of embodiment 20 wherein an rAAV vector prepared from the capsid protein has an at least 2 fold, 5 fold, 10 fold, 15 fold, 20 fold, or 25 fold greater transduction of CNS neurons than an rAAV vector prepared from the capsid protein without the peptide insert or the reference capsid protein.
22. The rAAV capsid protein of any one of the preceding embodiments wherein an rAAV vector prepared from the capsid protein has reduced transduction of liver, heart or astrocytes than an rAAV vector prepared from the capsid protein without the peptide insert or the reference capsid protein.
23. The rAAV capsid protein of embodiment 22 wherein the rAAV vector prepared from the capsid protein has an at least 2 fold, 5 fold, 10 fold, 15 fold, 20 fold, 25 fold or 40 fold lower transduction of liver than an rAAV vector prepared from the capsid protein without the peptide insert or the reference capsid protein.
24. The rAAV capsid protein of any one of embodiments 19, 21, 22, or 23 wherein the reference capsid protein is AAV9 capsid or AAVhu.32 capsid.
25. A nucleic acid comprising a nucleotide sequence encoding the rAAV capsid protein of any one of the preceding embodiments or embodiments 42 to 48, or encoding an amino acid sequence sharing at least 80% identity therewith.
26. The nucleic acid of embodiment 25 which encodes the rAAV capsid protein of any one of the preceding embodiments.
27. A packaging cell capable of expressing the nucleic acid of embodiment 25 or 26 to produce AAV vectors comprising the capsid protein encoded by said nucleotide sequence.
28. A rAAV vector comprising the capsid protein of any one of embodiments 1-24 or embodiments 42 to 48.
29. The rAAV vector of embodiment 28, further comprising a rAAV genome comprising a transgene flanked by AAV ITR sequences.
30. A pharmaceutical composition comprising the rAAV vector of embodiment 28 or 29 and a pharmaceutically acceptable carrier.
31. A method of delivering a transgene to a cell, said method comprising contacting said cell with the rAAV vector of embodiment 28 or 29; or the rAAV vector of embodiment 28 or 29 for use in delivering a transgene to a cell, wherein said cell is contacted with the vector.
32. A method of delivering a transgene to a target tissue of a subject in need thereof, said method comprising administering to said subject the rAAV vector of embodiment 28 or 29; or the rAAV vector of embodiment 28 or 29 for use in delivering a transgene to a target tissue of a subject in need thereof, wherein the vector is administered to said subject.
33. The method, or rAAV vector for use, according to embodiment 32, wherein said rAAV vector is administered systemically, intravenously, intrathecally, intra-nasally, intra-peritoneally, intravitreally, via lumbar puncture or via the cisterna magna.
34. The method, or rAAV vector for use, according to embodiment 32 or embodiment 33, wherein said target tissue is muscle, retina or CNS.
35. A method of making a recombinant AAV (rAAV) vector library comprising
   (i) producing a starting plasmid have a gene expression cassette comprising a nucleic acid sequence encoding an AAV capsid protein, wherein a stop codon is placed at a target insertion site within the nucleic acid sequence encoding the AAV capsid protein;
   (ii) providing a population of nucleic acids which encode a repertoire of peptides to produce a peptide library;
   (iii) creating individual plasmids based on the starting plasmid 1) each comprising the nucleic acid encoding the AAV capsid with a nucleic acid encoding a random peptide from the peptide library inserted at the target insertion site of the nucleic acid encoding the capsid protein thus replacing the stop codon, and 2) each plasmid further containing a nucleic acid encoding a barcode for identification of the nucleic acid encoding the capsid and peptide insert placed before the 5′- or after the 3′-end of the nucleic acid encoding the capsid;
   (iv) collecting the individual plasmids;
   (v) transfecting a population of cells with the collection of individual plasmids and one or more plasmids carrying an rAAV genome comprising a transgene and necessary genes;
   (vi) culturing the population of transfected cells under appropriate conditions to produce a collection of rAAV vectors each comprising the AAV capsid having the peptide insert encapsidating the rAAV genome comprising the transgene; and
   (vii) harvesting the rAAV vector library.
36. The method of embodiment 35, wherein the capsid gene encodes an AAV1 capsid protein (SEQ ID NO: 139), an AAV4 capsid protein (SEQ ID NO: 142), an AAV5 capsid protein (SEQ ID NO: 143), an AAV8 capsid protein (SEQ ID NO: 146), an AAV9 capsid protein (SEQ ID NO: 151), an AAV9.AAA capsid protein (SEQ ID No: 158), AAVhu.32 (SEQ ID NO: 148), AAVhu.32.AAA (SEQ ID NO. 267), an AAV3B capsid protein (SEQ ID NO: 154) or an AAVhu37 capsid protein (SEQ ID NO: 153).
37. The method of any one of embodiments 34 to 36, wherein the target insertion site is at or after an amino acid residue within VR-4 or VR-8 of the capsid protein
38. The method of embodiment 37, wherein after the target insertion site is after an amino acid corresponding to 5454 of AAV9 (SEQ ID NO: 151).
39. The method of any one of embodiments 34 to 38 wherein the peptide is a 7 amino acid peptide.
40. An rAAV vector library made by the method of any one of embodiments 34 to 39.
41. An rAAV vector library of embodiment 40 wherein at least 80%, 85%, 90%, 95%, 98%, 99% or 100% of the rAAV vectors in the library have a capsid with a peptide insert.
42. A recombinant adeno-associated virus (rAAV) capsid protein comprising a peptide insertion of at least 4 and up to 7 contiguous amino acids selected from the group consisting of SEQ ID NOs: 1-138, wherein said peptide insertion is surface exposed when said capsid protein is packaged as an AAV particle, and
   wherein said capsid protein is a wild-type capsid protein or a variant capsid protein having up to three amino acid substitutions, and
   wherein an rAAV vector prepared from the capsid protein comprising the peptide insertion provides enhanced tropism to a target tissue compared to an rAAV vector prepared from a capsid protein without the peptide insertion or a reference capsid protein.
43. The rAAV capsid protein of embodiment 42, wherein said capsid protein is serotype 5 (AAV5) (SEQ ID NO: 143) or a capsid protein that has 90%, 95%, or 99% sequence identity thereto.
44. The rAAV capsid protein of embodiment 42 or 43, wherein said peptide insertion occurs immediately after one of amino acid residues 574 to 584 of the AAV5 capsid protein.
45. The rAAV capsid protein of any one of embodiments 42 to 44, wherein said peptide insertion occurs immediately after amino acid residue 578 of the AAV capsid protein.
46. The rAAV capsid protein of any one of embodiments 42 to 45, wherein said peptide insertion is RHVSASD (SEQ ID NO: 113), VRSDRDQ ((SEQ ID NO: 114) or TVVTSIN (SEQ ID NO: 115).
47. The rAAV capsid protein of any one of embodiments 42 to 46 wherein the reference capsid protein is AAV5, AAV9, AAV9.AAA, AAVhu32, or AAVhu32.AAA.
48. The rAAV capsid protein of any one of embodiments 42 to 47 which has an amino acid sequence of SEQ ID No. 376, 377 or 378.

### 4. BRIEF DESCRIPTION OF THE FIGURES

FIG. 1 depicts alignment of AAVs 1-9, hu31, hu32, and rh10 capsid sequences highlighting the VR-IV insertion site for these capsids (see VR4 corresponding to amino acids 451 to 461 of the AAV9 capsid protein and VR8 corresponding to amino acids 585 to 593 of the AAV9 capsid protein). A Clustal Multiple Sequence Alignment of AAV capsids was performed and illustrates that amino acid substitutions (shown in bold in the bottom rows) can be made to AAV9, AAVhu32 or other capsids by “recruiting” amino acid residues from the corresponding position of other aligned AAV capsids. Sequences shown in gray are hypervariable regions (HVR). The amino acid sequences of the AAV capsids are assigned SEQ ID NOs as follows: AAV1 is SEQ ID NO: 139; AAV2 is SEQ ID NO: 140; AAV3 is SEQ ID NO: 141; AAV4 is SEQ ID NO: 142; AAV5 is SEQ ID NO: 143; AAV6 is SEQ ID NO: 144; AAV7 is SEQ ID NO: 145; AAV8 is SEQ ID NO: 146; AAV9 is SEQ ID NO: 151; AAVrh10 is SEQ ID NO: 152; hu31 is SEQ ID NO: 147; and hu32 is SEQ ID NO: 148.

FIG. 2 illustrates a protein model of variable region four and eight of the adeno-associated virus type 9 (AAV9 VR-IV and AAV9 VR-VIII, respectively).

FIG. 3 depicts a representative genome construct of the capsid gene for use in construction of rAAV libraries having from 5′ to 3′: 5′-inverted terminal repeat (ITR), CMV enhancer-promoter, Rep intron, the AAV Cap gene of interest, polyA sequence, and 3′-ITR. The illustration depicts insertion of a random peptide library in the place of a stop codon (see arrow) that was inserted into the Cap gene variable region before construction of the library (to reduce expression of wildtype sequence in the library).

FIG. 4 demonstrates the PCR amplicon obtained from libraries A1 to G1 used for NGS analysis of library diversity.

FIGS. 5A-5B show graphed results of the % wildtype and % stop codon sequences by NGS analysis of plasmid and vector libraries. FIG. 5A depicts a library having higher parental vector levels following vector production (vector % wt), compared to the percent parental plasmid (plasmid % wt) in the initial plasmid preparation of the library. FIG. 5B depicts NGS analysis of a library in which a stop codon was inserted into the template plasmid before plasmid library construction. The results indicate a reduction in stop parental sequence in the vector library (vector % stop) compared to the initial plasmid library(plasmid % stop).

FIGS. 6A-6B. Liquid chromatography-mass spectrometry (LC-MS) of VP3 proteins following generation of an AAV5 vector library with or without stop codon in template.

FIG. 7 depicts the peptides analyzed by NGS in various tissues with various shading representing enrichment score. SEQ ID NOs: 1-24 (top to bottom) are listed on the left side of the density plot.

FIGS. 8A-8C depicts the relative abundance (RA), represented as fold change, for certain rAAV vectors with peptide insertions (24=peptide 24=SEQ ID NO: 24, and so on), relative to the control parental vector, AAV9.AAA. Comparisons of RA are made in heart (FIG. 8A), skeletal muscle (FIG. 8B), and liver (FIG. 8C) tissue.

FIGS. 9A-9B depicts the nRAAFI in NHP for certain rAAV vectors with peptide insertions (NVG01 to NVG14) compared to AAV9, AAVhu32, and AAV5 in skeletal muscle (A) and heart (B).

FIGS. 10A-10B are bar graphs depicting the mRNA/DNA in NHP of certain rAAV vectors with peptide insertions (NVG01 to NVG14) compared to AAV9, AAVhu32, and AAV5 in skeletal muscle (A) and heart (B).

FIGS. 11A-11B are bar graphs depicting the nRAAFI of mRNA (A) and DNA (B) in liver of NHP injected with pooled AAV with peptide insertions (NVG01 to NVG14).

FIGS. 12A-C are bar graphs showing the relative abundance (RA) of mRNA in skeletal muscle (A) liver (B) or the ratio of mRNA in muscle/liver (C) in individual NHP (numbered 1001, 1002, 1003, 4001, and 4003) injected with either AAV9 or AAV9.AAA.NVG07 (SEQ ID NO: 8).

FIGS. 13A-B are bar graphs showing the RNA (TdTom/TBP) or DNA (TdTom GC/diploid genome) of CB57Bl6 mice injected with 1E14 GC/kg IV dose of AAV (AAVhu.32, AAV9.AAA.NVG07, or AAVhu.32.AAA.NVG09) expressing the CAG.TdTomato transgene at three weeks. RNA and DNA were measured in the heart, gastrocnemius, quadricep, bicep, tibialis anterior (TA), brain, liver and diaphragm.

### 5. DETAILED DESCRIPTION

Provided are recombinant adeno-associated viruses (rAAVs) having capsid proteins engineered to include amino acid sequences that confer and/or enhance desired properties, such as tissue targeting, transduction and integration of the rAAV genome. In particular, provided are engineered capsid proteins comprising peptide insertions of 4 to 7 contiguous amino acids, from random peptide libraries, inserted within or near variable region IV (VR-IV) or VR-VIII of the virus capsid, such that the peptide insertion is surface exposed when the capsid protein is packaged as an AAV particle. Also provided are recombinant capsid proteins, and libraries of the individual rAAVs comprising them, that have inserted peptides that target specific tissues and/or promote rAAV cellular uptake, transduction and/or transgene expression, for example, see Table 17.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “subject”, “host”, and “patient” are used interchangeably. As used herein, a subject is a mammal such as a non-primate (e.g., cows, pigs, horses, cats, dogs, rats etc.) or a primate (e.g., monkey and human), or, in certain embodiments, a human.

“Library” or “libraries” generally refer to a repertoire of capsid genes (each unique and usually placed recombinantly into a vehicle, such as in a plasmid) or rAAV vectors produced from the unique capsids.

As used herein, the term “conservative amino acid substitution” means substitutions made in accordance with Tables A and B.

TABLE A

| Amino Acid Substitutions | |
|---|---|
| | Non-limiting Exemplary |
| Original Residue | Conservative Substitutions |
| Ala | Ser |
| Arg | Gly; Gln; Lys |
| Asn | Gln; His |
| Asp | Glu |
| Cys | Ser |
| Gln | Asn; Lys |
| Glu | Asp |
| Gly | Ala |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

TABLE B

| Amino Acid Abbreviations | |
|---|---|
| Amino Acid | Abbreviations |
| Alanine | Ala (A) |
| Allosoleucine | AIle |
| Arginine | Arg (R) |
| Asparagine | Asn (N) |
| Aspartic Acid | Asp (D) |
| Cysteine | Cys (C) |
| Glutamic Acid | Glu (E) |
| Glutamine | Gln (Q) |
| Glycine | Gly (G) |
| Histidine | His (H) |
| Isolelucine | Ile (I) |
| Leucine | Leu (L) |
| Lysine | Lys (K) |
| Phenylalanine | Phe (F) |
| Praline | Pro (P) |
| Pyroglutamic Acid | PGlu (U) |
| Serine | Ser (S) |
| Threonine | Thr (T) |
| Tyrosine | Tyr (Y) |
| Tryptophan | Trp (W) |
| Valine | Val (V) |

It is understood that one way to define the variants and derivatives of the disclosed capsid proteins herein is to define them in terms of homology/identity to specific known sequences. Specifically disclosed are variants of capsids herein disclosed which have at least, 70% or at least 75% or at least 80% or at least 85% or at least 90% or at least 91% or at least 92% or at least 93% or at least 94% or at least 95% or at least 96% or at least 97% or at least 98% or at least 99% identity to the capsid sequences specifically recited herein. Those of skill in the art readily understand how to determine the identity of two proteins. Variants of capsids described herein may have 1, 2, 3, 4, 5, 6, 7, 8, 9, 10, 15 or 20 amino acid substitutions, including conservative amino acid substitutions. Variants of the 4 to 7 amino acid peptides described herein include peptides having 1, 2, or 3 amino acid substitutions, including conservative amino acid substitutions.

The term “therapeutic agent” refers to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

As used herein, the term “prophylactic agent” refers to any agent which can be used in the prevention, delay, or slowing down of the progression of a disease or disorder, where the disease or disorder is associated with a function to be provided by a transgene. As used herein, a “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto.

### 5.2. Recombinant AAV Capsids and Vectors

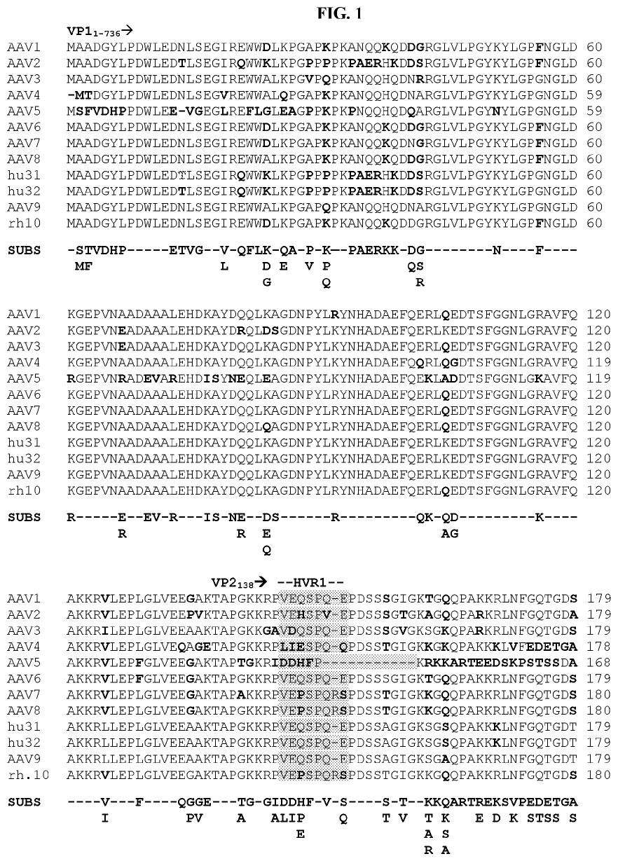

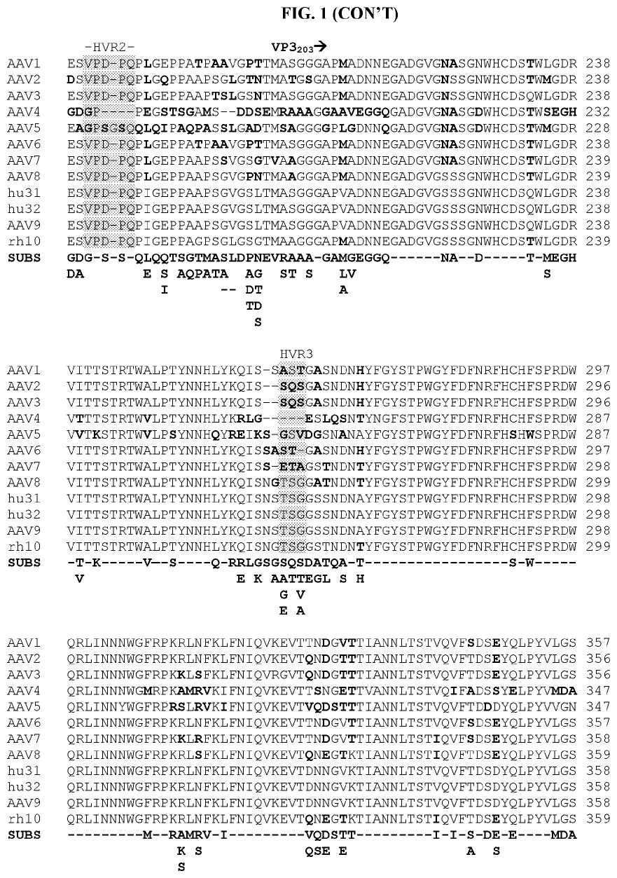

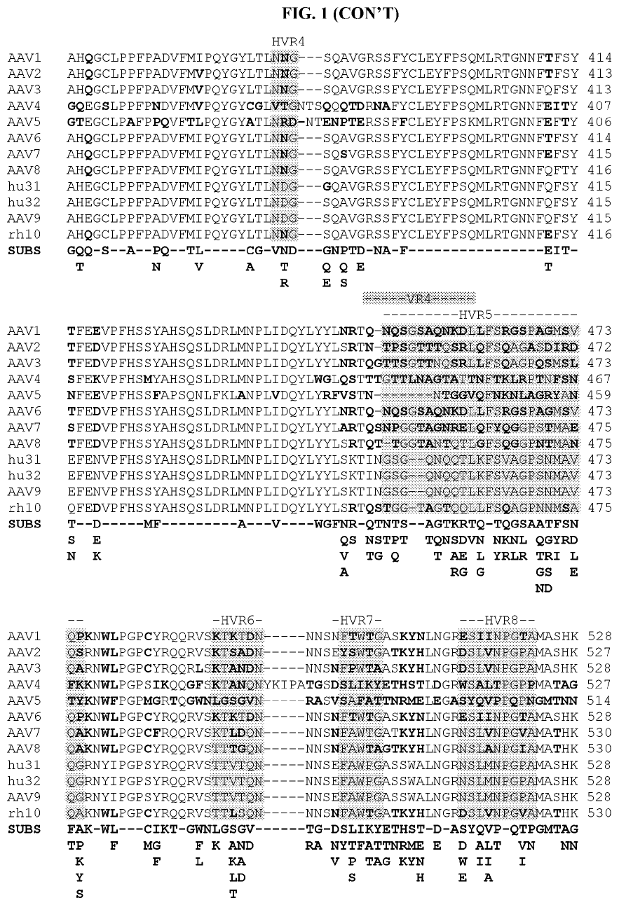

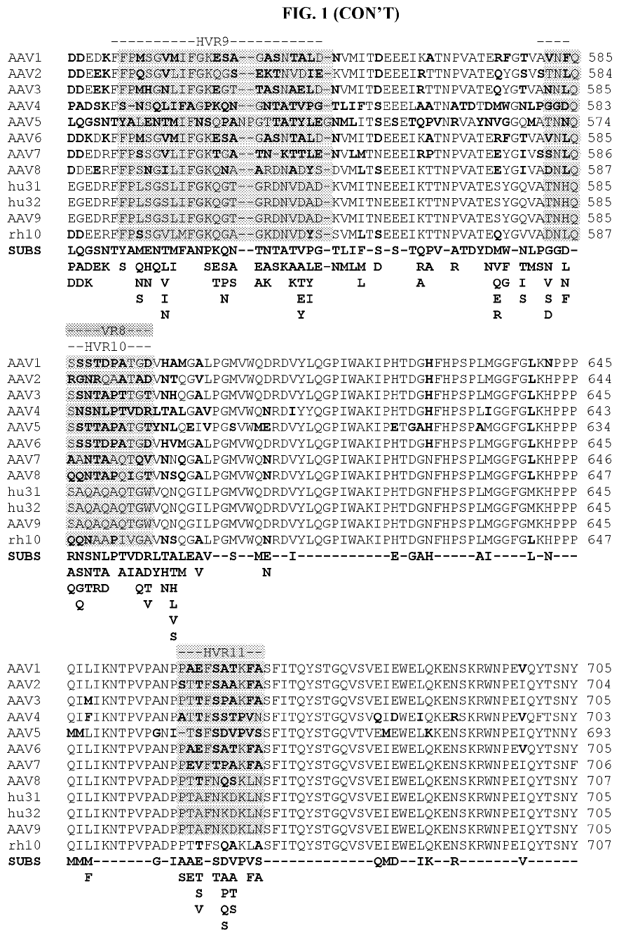

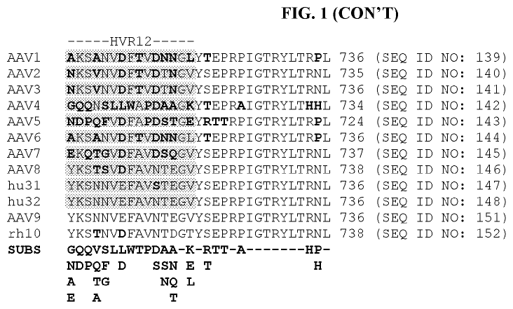

One aspect relates to capsid protein libraries and the recombinant adeno-associated virus (rAAV) vectors thereof, the capsid proteins within the library engineered to comprise a peptide insertion from a random peptide library, wherein the peptide is not an AAV protein or peptide fragment thereof, where the peptide insertion is surface exposed when packaged as an AAV particle. In some embodiments, the peptide insertion occurs within (i.e., between two amino acids without deleting any capsid amino acids) variable region IV (VR-IV) of an AAV9 capsid or AAV9.AAA capsid, or a corresponding region for another type AAV capsid (see alignment in FIG. 1). In some embodiments, the peptide insertion occurs within (i.e., between two amino acids without deleting any capsid amino acids) variable region VIII (VR-VIII) of an AAV9 capsid or AAV9.AAA, or a corresponding region of a capsid for another AAV type (see alignment in FIG. 1). In some embodiments, the peptide insertion is from a heterologous protein or domain (that is not an AAV capsid protein or domain), which directs the rAAV particles to target tissues and/or promote rAAV uptake, transduction and/or transgene expression. Also provided are nucleic acids encoding the engineered capsid proteins and variants thereof, packaging cells for expressing the nucleic acids to produce rAAV vectors, rAAV vectors further comprising a transgene, and pharmaceutical compositions of the rAAV vectors, as well as methods of using the rAAV vectors to deliver the transgene to a target cell type or target tissue of a subject in need thereof.

In the various embodiments, the target tissue may be muscle tissue, such as skeletal muscle or heart muscle, neural tissue, bone, kidney, the eye/retina, or endothelial tissue, and the capsid with the peptide insertion specifically recognizes and/or binds to and/or homes to that tissue, or for example, one or more specific cell types, such as within the target tissue, or cellular matrix thereof. In particular, peptides that can target rAAVs to muscle tissue, including skeletal muscle and heart, can be useful for delivering therapeutics for treating muscle disorders. In embodiments, rAAV capsids containing the engineered capsid protein with the peptide insert have increased uptake, transduction, and/or transgene expression than the parental capsid or a reference capsid, including AAV9, AAV9.AAA, AAVhu.32, AAVhu.32.AAA or AAV5. The engineered capsids may further have reduced transduction in tissues such as liver, heart, or astrocytes or other tissues relative to the targeted tissue or relative to transduction with the parental capsid or a reference capsid, including AAV9, AAV9.AAA, AAVhu.32, AAVhu.32.AAA or AAV5.

In various embodiments, the target tissue may be neural tissue, and particularly neurons in the brain. In embodiments, the peptide insertions target rAAVs preferentially to neurons over astrocytes compared to rAAVs that do not have the peptide insertion.

In embodiments either the parental capsid and/or the peptide insert detarget the rAAV vector prepared from the engineered capsid protein from one or more tissue types, including liver.

### 5.2.1 rAAV Vectors with Peptide Insertions

A peptide insertion described as inserted “at” a given site refers to insertion immediately after, that is having a peptide bond to the carboxy group of, the residue normally found at that site in the wild type virus. For example, insertion at S454 in AAV9 means that the peptide insertion appears between S454 and the consecutive amino acid (G455) in the AAV9 wildtype capsid protein sequence (SEQ ID NO: 151). In embodiments, there is no deletion of amino acid residues at or near (within 5, 10, 15 residues or within the structural loop that is the site of the insertion) the point of insertion.

In embodiments, the capsid protein is an AAV9 capsid protein (SEQ ID NO: 151, or a capsid protein having an amino acid sequence that is 90%, 95%, or 99% identical to SEQ ID NO: 151) or an AAV9.AAA capsid protein and the insertion occurs immediately after at least one of the amino acid residues 451 to 461. In embodiments, the peptide insertion occurs immediately after amino acid 1451, N452, G453, S454, G455, Q456, N457, Q458, Q459, T460, or L461 of the AAV9 capsid (amino acid sequence SEQ ID NO: 151). In certain embodiments, the peptide is inserted between residues S454 and G455 of AAV9 capsid protein or between the residues corresponding to S454 and G455 of an AAV capsid protein other than an AAV9 capsid protein (amino acid sequence SEQ ID NO: 151). In embodiments, the capsid protein is an AAV9.AAA capsid protein and the insertion occurs immediately after at least one of the amino acid residues 451 to 461. In embodiments, the peptide insertion occurs immediately after amino acid 1451, N452, G453, S454, G455, Q456, N457, Q458, Q459, T460, or L461 of the AAV9.AAA capsid (amino acid sequence SEQ ID NO: 123). In certain embodiments, the peptide is inserted between residues S454 and G455 of AAV9.AAA capsid protein or between the residues corresponding to S454 and G455 of an AAV.AAA capsid protein other than an AAV9 capsid protein (amino acid sequence SEQ ID NO: 123).

In embodiments, the capsid protein is an hu.32 capsid protein (SEQ ID NO: 148, or a capsid protein having an amino acid sequence that is 90%, 95%, or 99% identical to SEQ ID NO: 148) or an AAVhu.32.AAA capsid protein and the insertion occurs immediately after at least one of the amino acid residues 451 to 461. In embodiments, the peptide insertion occurs immediately after amino acid 1451, N452, G453, S454, G455, Q456, N457, Q458, Q459, T460, or L461 of the AAVhu.32 capsid (amino acid sequence SEQ ID NO: 148). In certain embodiments, the peptide is inserted between residues S454 and G455 of AAVhu.32 capsid protein. In embodiments, the capsid protein is an AAVhu.32.AAA capsid protein and the insertion occurs immediately after at least one of the amino acid residues 451 to 461. In embodiments, the peptide insertion occurs immediately after amino acid 1451, N452, G453, S454, G455, Q456, N457, Q458, Q459, T460, or L461 of the AAVhu.32.AAA capsid (amino acid sequence SEQ ID NO: 148). In certain embodiments, the peptide is inserted between residues S454 and G455 of AAVhu.32.AAA capsid protein.

In embodiments, the capsid protein is an AAV5 capsid protein (SEQ ID NO: 143, or a capsid protein having an amino acid sequence that is 90%, 95%, or 99% identical to SEQ ID NO: 143) and the insertion occurs immediately after at least one of the amino acid residues 442 to 447. In embodiments, the peptide insertion occurs immediately after amino acid N442, N443, T444, G445, G446, V447 of the AAV5 capsid (amino acid sequence SEQ ID NO: 143). In embodiments, the peptide is inserted after a residue within VR8 of the AAV5 capsid protein In embodiments, the peptide is inserted after residue 578 of AAV5 capsid protein.

In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), and serotype rh74 (AAVrh74, versions 1 and 2) (see FIG. 1) or a capsid that is at least 90%, 95% or 99% identical in amino acid sequence to the amino acid sequence of the VP1, VP2, or VP3 of the foregoing capsid proteins, and the insertion occurs immediately after an amino acid residue corresponding to at least one of the amino acid residues 451 to 461 of AAV9. The alignments of these different AAV serotypes, as shown in FIG. 1, indicates “corresponding” amino acid residues in the different capsid amino acid sequences such that a “corresponding” amino acid residue is lined up at the same position in the alignment as the residue in the reference sequence. In some embodiments, the peptide insertion occurs immediately after one of the amino acid residues within: 450-459 of AAV1 capsid (SEQ ID NO: 139); 449-458 of AAV2 capsid (SEQ ID NO: 140); 449-459 of AAV3 capsid (SEQ ID NO: 141); 443-453 of AAV4 capsid (SEQ ID NO: 142); 442-445 of AAV5 capsid (SEQ ID NO: 143); 450-459 of AAV6 capsid (SEQ ID NO: 144); 451-461 of AAV7 capsid (SEQ ID NO: 145); 451-461 of AAV8 capsid (SEQ ID NO: 146); 451-461 of AAV9 capsid (SEQ ID NO: 151); 451-461 of AAVhu.32 capsid (SEQ ID NO: 148); 452-461 of AAVrh10 capsid (SEQ ID NO: 152); 452-461 of AAVrh20 capsid (SEQ ID NO: 155); 452-461 of AAVhu.37 (SEQ ID NO: 149); 452-461 of AAVrh74 (SEQ ID NO: 156 or SEQ ID NO: 157); or 452-461 of AAVrh39 (SEQ ID NO: 150), in the sequences depicted in FIG. 1. In certain embodiments, the rAAV capsid protein comprises a peptide insertion immediately after (i.e., C-terminal to) amino acid 588 of AAV9 capsid protein (having the amino acid sequence of SEQ ID NO: 151 and see FIG. 1) or AAV9.AAA (having SEQ ID NO: 158), where said peptide insertion is surface exposed when the capsid protein is packaged as an AAV particle. In other embodiments, the rAAV capsid protein has a peptide insertion that is not immediately after amino acid 588 of AAV9 or AAV9.AAA, or corresponding to amino acid 588 of AAV9.

Also provided are AAV vectors comprising the engineered capsids. In some embodiments, the AAV vectors are non-replicating and do not include the nucleotide sequences encoding the rep or cap proteins (these are supplied by the packaging cells in the manufacture of the rAAV vectors). In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more serotypes. In some embodiments, rAAV particles comprise a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. These engineered AAV vectors may comprise a genome comprising a transgene encoding a therapeutic protein.

In embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In embodiments, the AAV for use in compositions and methods herein is any AAV disclosed in U.S. Pat. No. 9,585,971, such as AAV-PHP.B. In embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) may be used. In certain embodiments, a self-complementary vector, e.g., scAAV, may be used (see, e.g., Wu, 2007, Human Gene Therapy, 18(2):171-82; McCarty et al, 2001, Gene Therapy, 8(16):1248-1254; U.S. Pat. Nos. 6,596,535; 7,125,717; and 7,456,683, each of which is incorporated herein by reference in its entirety).

Generally, the peptide insertion is sequence of contiguous amino acids from a heterologous protein or domain thereof. The peptide to be inserted typically is long enough to retain a particular biological function, characteristic, or feature of the protein or domain from which it is derived. The peptide to be inserted typically is short enough to allow the capsid protein to form a coat, similarly or substantially similarly to the native capsid protein without the insertion. In preferred embodiments, the peptide insertion is from about 4 to about 30 amino acid residues in length, about 4 to about 20, about 4 to about 15, about 5 to about 10, or about 7 amino acids in length. The peptide sequences for insertion are at least 4 amino acids in length and may be 5, 6, 7, 8, 9, 10, 11, 12, 13, 14, or 15 amino acids in length. In some embodiments, the peptide sequences are 16, 17, 18, 19, or 20 amino acids in length. In embodiments, the peptide is no more than 7 amino acids, 10 amino acids or 12 amino acids in length.

A “peptide insertion from a heterologous protein” in an AAV capsid protein refers to an amino acid sequence that has been introduced into the capsid protein and that is not native to the AAV serotype capsid into which it is inserted.

As used herein, the terms “homing” and “targeting” are used interchangeably. These peptides may also or alternatively promote rAAV cell uptake, transduction and/or genome integration in cells of the target tissue.

Examples of peptides for use as peptide insertions at any of the AAV capsid sites described herein are presented in Tables 4, 5, 14, 15 and 16 below in the Examples (including the peptides having amino acid sequences of SEQ ID Nos 1-138), and include at least 4 amino acid contiguous portions thereof, or 7 amino acid contiguous portions thereof (and includes variants having 1, 2 or 3 amino acid substitutions, including conservative amino acid substitutions) and have the functional attribute of the peptide being inserted alters the properties of the capsid, particularly its tropism. In certain embodiments, the recombinant AAV capsids and AAV vectors are engineered to include a peptide, or at least 4, 5, 6, or 7 amino acid contiguous portion thereof, from any of Tables 4, 5, 14, 15 and 16 below (including peptides of amino acid sequences of SEQ ID Nos 1-138), inserted into the AAV capsid sequence in such a way that the peptide insertion is displayed. In other embodiments, the peptides are inserted after an amino acid residue at positions 138, 262-273, 451-461, or 585-593 of the amino acid sequence of the AAV9 capsid (SEQ ID NO: 151) or of the AAV9.AAA capsid (SEQ ID NO: 158) a position corresponding thereto in any other AAV serotype (see FIG. 1 for capsid sequence alignments).

In another aspect, provided are heterologous peptide insertion libraries. A heterologous peptide insertion library refers to a collection of rAAV vectors that carry the same random peptide insertion at the same insertion site in the virus capsid to make the particular library, e.g., at a position within a given variable region of the capsid. Generally, the capsid proteins used comprise AAV genomes that contain modified rep and cap sequences to prevent the replication of the virus under conditions in which it could normally replicate (co-infection of a mammalian cell along with a helper virus such as adenovirus). The members of the peptide insertion libraries may then be assayed for functional display of the peptide on the rAAV surface, tissue targeting and/or gene transduction. Enhanced properties or desirable properties may be assessed upon comparison with the parental capsid from which the insertion library was made.

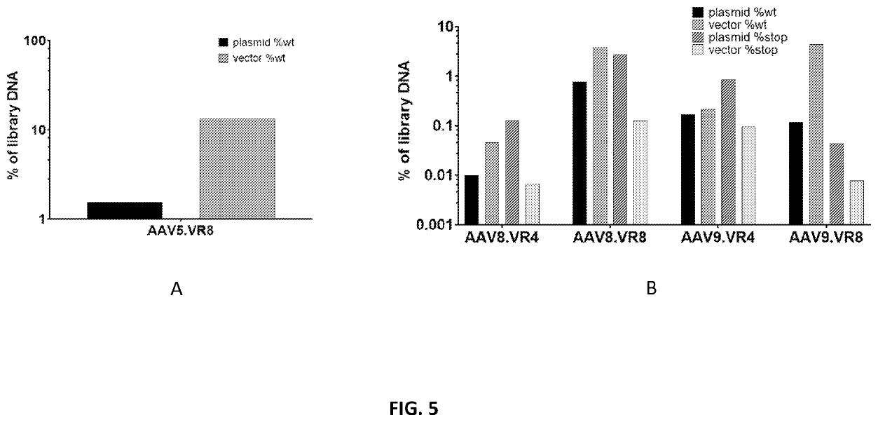

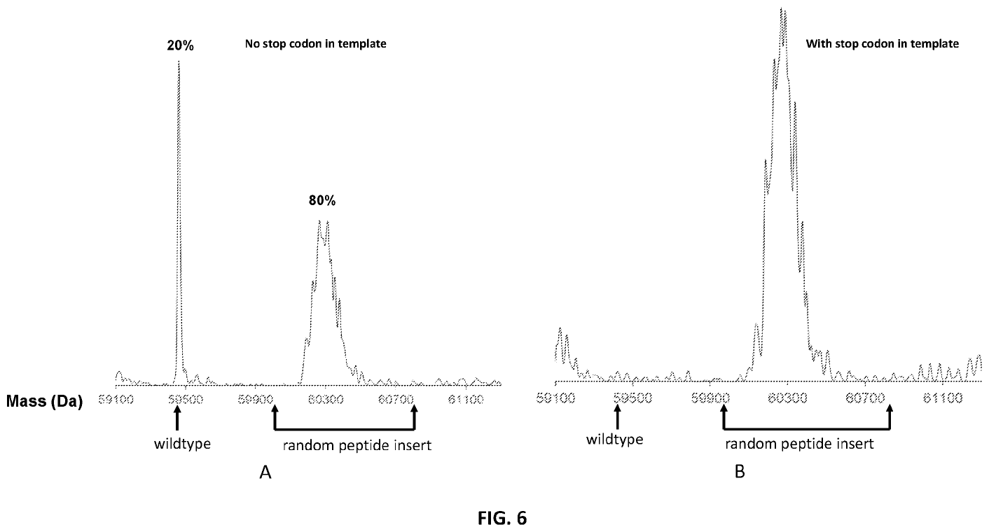

Provided are peptide insertion libraries and methods of making these libraries. An exemplary method of producing a capsid library with peptide inserts is described herein in Example 2 and such libraries are screened herein. In such libraries, the nucleic acid encoding the parental capsid has a stop codon at the target insertion site for the population of nucleic acids encoding the peptides such that a capsid protein will only be expressed if the peptide insert is present, otherwise, translation will terminate prematurely. Production of the populations of rAAVs from the expression constructs results in an rAAV population with a high percentage of capsids with peptide inserts (including 80%, 85%, 90%, 95%, 98%, 99% or even 100%) (see, for example, FIGS. 5A, 5B, 6A, and 6B).

Also provided are methods of making the capsid libraries having the variable peptide inserts. In embodiments, the method comprises (1) providing a starting plasmid that contains a gene expression cassette comprising a nucleic acid sequence encoding an AAV capsid, wherein a stop codon is placed at the target insertion site within the capsid gene, (2) providing a repertoire of nucleic acids encoding randomized peptides to produce a peptide library; (3) creating individual plasmids based on the starting plasmid a) each having a nucleic acid encoding a random peptide from the peptide library inserted at the target insertion site of the capsid gene thus replacing the stop codon, and b) each encoding a barcode for identification of the capsid gene having the insert placed before the 5′- or after the 3′-end of the capsid gene; (4) collecting the individual plasmids to form a population or collection of plasmids encoding the capsids with peptide inserts and transfecting cells with the collection of plasmids and with plasmids encoding a recombinant rAAV genome containing a transgene, including one that is detectable, and constructs having the necessary genes to produce, under appropriate conditions, a collection of rAAV vectors encapsidating the rAAV genome containing the transgene, wherein the rAAV vectors have capsids with the encoded capsid protein containing a library peptide insert. In embodiments, the parental AAV is AAV9, AAV9.AAA, AAVhu.32, AAVhu.32.AAA, or AAV5 or any other suitable AAV serotype. The insertion site may be in VR-IV, including immediately after one of amino acids 451-461 of AAV9 or corresponding to one of those residues or in VR-VIII, including immediately after amino acid 588 of AAV9 or corresponding to that position in a different AAV capsid type (see FIG. 1 for alignment). The peptides may be 4, 5, 6, or 7 amino acids in length.

In embodiments, the rep gene is on a separate expression plasmid from the plasmid encoding the cap gene with the inserts.

The library of modified capsids is harvested from these cells. In embodiments, the rAAV library population produced has high levels of capsids having the peptide inserts, including 85%, 90%, 95% or 98%, 99% or even 100%.

### 5.2.2 Capsids Containing Muscle-Homing and Other Targeting Peptides

The present inventors also have surprisingly discovered peptides that inserted into rAAV vectors “re-target” or enhance targeting properties of such AAV vectors to specific tissues, organs, or cells; in particular, providing peptides that cause rAAV vectors to target muscle tissue and/or other target tissues of interest, such as retinal tissue, or to cross the blood-brain barrier and target neural tissue of the CNS. This can provide enhanced transport of rAAV particles encapsidating a transgene for optimizing distribution of the vector upon administration to the body. Such peptides, and modified vectors, are described below.

Another aspect of the present invention relates to capsid proteins selected from the random peptide insertion libraries comprising peptide insertions selected to confer or enhance muscle-homing properties, or “muscle tropism”, including homing to muscle tissue, muscle cells or muscle cell matrix. Also included are capsids and rAAV vectors having capsids comprising these peptide-containing capsid proteins. In other aspects, the peptides may target other tissues, such as, retinal, CNS, including preferentially neurons within the CNS and may also detarget tissues such as liver, heart or astrocytes.

In certain embodiments, the peptide insertion consists of 4 to 7 contiguous amino acids (that is, 4, 5, 6, or 7 contiguous amino acids) of a peptide sequence of Tables 4, 5, 14, 15 and 16 (SEQ ID Nos 1-138). The peptides disclosed herein were identified by screening libraries of peptides inserted in AAV capsids, which are screened for properties such as tropism for muscle tissue (skeletal and/or heart), retinal tissue, CNS neuronal tissue and other tissues, for example, through mouse and NHP biodistribution studies as described in the examples. In embodiments, the capsids having the peptide insertions have increased tropism for a target tissue, including muscle, retinal tissue, CNS, including neuronal tissue, relative to the parental capsid (that is having the capsid protein without the peptide insert) or a reference capsid, such as AAV9, AAV9.AAA, AAVhu.32, or AAVhu.32.AAA. The capsids with the peptide inserts may also distribute to liver tissue at levels less than target tissues such as muscle, retina, CNS or other target tissue and/or less than a reference capsid, such as AAV9 or AAVhu.32.

In embodiments, the peptides that may be inserted into capsid proteins include those listed in tables 4, 5, 14, 15, and 16 and include peptides having amino acid sequences of 4, 5, 6, or 7 contiguous amino acids of one of the amino acid sequences of SEQ ID Nos: 1-134. In embodiments, the peptide is a 4, 5, 6, or 7 contiguous amino acid sequence of one of SGTVIRS (SEQ ID NO: 1), TRQYVPG (SEQ ID NO: 4), VQVGRTS (SEQ ID NO: 8), RGAVQKV (SEQ ID NO: 13), GQVHQAR (SEQ ID NO: 32), TSGGQIR (SEQ ID NO: 38), HMGHSGK (SEQ ID NO: 88), MRAVSQL (SEQ ID NO: 109), PRQYVPG (SEQ ID NO: 110), RSSSGR (SEQ ID NO: 111), VVKSTKS (SEQ ID NO: 112), RHVSASD (SEQ ID NO: 113), VRSDRDQ (SEQ ID NO: 114), or TVVTSIN (SEQ ID NO: 115). In embodiments, the peptide is inserted in VR-IV (including immediately after one of amino acids 451-461 or 454 of AAV9, AAV9.AAA, AAVhu.32, AAVhu.32.AAA).

In embodiments, the peptide is a 4, 5, 6, or 7 contiguous amino acid sequence of one of AQVGRAS (SEQ ID NO: 24), SVTSVRV (SEQ ID NO: 37), SIAKNSA (SEQ ID NO: 76), DGRRIGV (SEQ ID NO: 116), TSGERRG (SEQ ID NO: 117), PSSVQHR (SEQ ID NO: 118), SSSVQHR (SEQ ID NO: 119), SGMQERR (SEQ ID NO: 120), LERGNLE (SEQ ID NO: 121), YRDVRQT (SEQ ID NO: 122), PSAVQHR (SEQ ID NO: 123), YVGGRAV (SEQ ID NO: 124), IGSRGVA (SEQ ID NO: 125), SDVSRPR (SEQ ID NO: 126), GSVRQAA (SEQ ID NO: 127), QSPHTSQ (SEQ ID NO: 128), or ASQAYHG (SEQ ID NO: 128). In embodiments, the peptide is inserted in VR-IV (including immediately after one of amino acids 451-461 or 454 of AAV9, AAV9.AAA, AAVhu.32, AAVhu.32.AAA).

In embodiments, provided are capsids having an amino acid sequence of SEQ ID NO 159-266 or 268-375 (see Table 17).

In other embodiments, the peptide is a variant of one of the amino acid sequences of SEQ ID Nos 1 to 134 which has 1, 2 or 3 amino acid substitutions, including conservative amino acid substitutions, while the peptide, when inserted into a capsid protein, retains its biological activity.

The inventors have also identified certain consensus sequences of peptides that when inserted into a capsid protein (for example in VR-IV of AAV9, AAV9.AAA, AAVhu.32, or AAVhu.32.AAA) exhibit increased muscle tropism, including AAV uptake, transduction, and/or transgene expression, relative to the parental capsid or a reference capsid such as AAV9, AAVhu.32, AAV9.AAA, or AAVhu.32.AAA. The peptides analyzed include the peptides of SEQ ID NOs: 5, 8, 24, 36, 44, 130, 131, 132, 133 or 134 (see Table 15, supra) all of which exhibited significant increase in muscle tropism compared to AAV9.AAA capsids. Accordingly, provided are capsids with peptide inserts, where the peptide is a 4, 5, 6, or 7 contiguous amino acid sequence of SEQ ID NO: 135, which is X₁-Q-V-X₂-X₃-X₄-X₅, wherein X₁ is V or A, X₂ is S, G, V or A, X₃ is R or H, X₄ is any amino acid and X₅ is S, G, V or A. In embodiments, provided are capsids with peptide inserts, where the peptide is a 4, 5, 6, or 7 contiguous amino acid sequence of SEQ ID NO: 136 is X₁-Q-V-X₂-X₃-X₄-X₅, wherein X₁ is V or A, X₂ is S, G, V or A, X₃ is R or H, X₄ is any amino acid and X₅ is S or A. In embodiments, provided are capsids with peptide inserts, where the peptide is a 4, 5, 6, or 7 contiguous amino acid sequence of SEQ ID NO: 137 is X₁-Q-V-X₂-X₃-X₄-X₅, wherein X₁ is V or A, X₂ is S, G, V or A, X₃ is R or H, X₄ is T, S, V, Y, A or P and X₅ is S, G, V or A. And, in embodiments, provided are capsids with peptide inserts, where the peptide is a 4, 5, 6, or 7 contiguous amino acid sequence of SEQ ID NO: 138 is X₁-Q-V-X₂-X₃-X₄-X₅, wherein X₁ is V or A, X₂ is S, G, V or A, X₃ is R or H, X₄ is T, S, V, Y, A or P and X₅ is S or A. In embodiments, the peptide is a 4, 5, 6, or 7 contiguous amino acid sequence of one of SEQ ID NOs: 5, 8, 24, 36, 44, 130, 131, 132, 133 or 134. In embodiments, the peptide is inserted in region VR-IV (immediately after one of positions 451-461, including immediately after 454 of or corresponding to AAV9) of one of capsids of AAV9, AAV9.AAA, AAVhu.32, or AAVhu.32.AAA. In embodiments, the capsid has an amino acid sequence of SEQ ID No. 263-266 (see Table 17).

As detailed herein, the peptides may be inserted into wild type or variant capsid protein amino acid sequences at positions such that the peptide is surface displayed when the capsid protein is incorporated into an AAV capsid, for example, at sites that allow surface exposure of the peptide, such as within variable surface-exposed loops, and, in more examples, sites described herein corresponding to VR-I, VR-IV, or VR-VIII, or may be inserted after the first amino acid of VP2, e.g. after amino acid 137 (AAV4, AAV4-4, and AAV5) or at amino acid 138 (AAV1, AAV2, AAV3, AAV3-3, AAV6, AAV7, AAV8, AAV9, AAV9e, rh.10, rh.20, rh.39, rh.74v1, rh.74v2, AAVhu.32 and hu.37) (FIG. 1). In embodiments, the capsid protein is an AAV9 capsid protein or an AAV9.AAA capsid protein (or a capsid protein with 90%, 95% or 99% amino acid sequence identity to AAV9 or AAV9.AAA) and the peptide insertion occurs immediately after at least one of (or corresponding to) the amino acid residues 451 to 461 of the AAV9 capsid. In embodiments, the capsid protein is an AAVhu.32 capsid protein or an AAVhu.32.AAA capsid protein (or a capsid protein with 90%, 95% or 99% amino acid sequence identity to AAVhu.32 or AAVhu.32.AAA) and the peptide insertion occurs immediately after at least one of (or corresponding to) the amino acid residues 451 to 461 of the AAVhu.32 capsid. In other embodiments, the capsid protein is from at least one AAV type selected from AAV1, AAV2, AAV3, AAV4, AAV5, AAV6, AAV7, AAV8, AAVrh8, AAV9, AAV9e, AAVrh10, AAVrh20, AAVhu.31, AAVhu.32, AAVhu.37, AAVrh39, and AAVrh74 (versions 1 and 2) (see, for example, FIG. 1) or a capsid protein that has 90%, 95% or 99% amino acid identity to the capsid protein of AAV1, AAV2, AAV3, AAV4, AAV5, AAV6, AAV7, AAV8, AAVrh8, AAV9, AAV9e, AAVrh10, AAVrh20, AAVhu.31, AAVhu.32, AAVhu.37, AAVrh39, and AAVrh74 (versions 1 and 2). In embodiments, the peptide insertion occurs immediately after an amino acid residue corresponding to at least one of the amino acid residues 451 to 461 of AAV9 (SEQ ID NO: 151) or, alternatively, 588 of AAV9. In embodiments, the peptide insertion occurs immediately after an amino acid residue corresponding to 578 of AAV5 (SEQ ID NO: 143). The alignments of different AAV serotypes, as shown in FIG. 1, indicates corresponding amino acid residues in the different amino acid sequences.

In embodiments, the capsid protein having the peptide insert is one of the capsid proteins in Table 17, with amino acid sequence SEQ ID Nos: 159 to 266 and 268-375. In embodiments, the capsid protein is 90%, 95% or 99% identical to SEQ ID Nos: 159 to 266 and 268-375, except that it is identical with respect to the peptide insert and retains its biological activity.

In certain embodiments, the peptide is inserted within VR-VIII of AAV5 (SEQ ID NO: 143), immediately after one of amino acids 574-584 (or to 582), including immediately after position 578 (see FIG. 1 for alignment). The peptides include any of peptides having an amino acid sequence of SEQ ID Nos: 1-134, and may include one of peptides having an amino acid sequence SEQ ID NO: 113, 114 or 115. In embodiments, the capsid has the amino acid sequence of SEQ ID NO: 376, 377 or 378.

In embodiments, the capsids with peptide inserts as described herein have increased tropism for target tissues, such as muscle, retina, CNS and other target tissue relative to the parental capsid, i.e., containing the capsid protein that is identical except that it does not have the peptide insert, or a reference capsid, which may include AAV9, AAV9.AAA, AAVhu.32, or AAVhu.32.AAA, or other capsid of interest that does not contain the peptide insert. In embodiments, the engineered capsid may have reduced tropism or is detargeted for tissues such as liver, including relative to target tissues, including muscle, retina, CNS or others, and/or relative to the parental capsid or a reference capsid, which may include AAV9, AAV9.AAA, AAVhu.32, or AAVhu.32.AAA. In embodiments, the reference capsid does not contain the 496NNN/AAA498 amino acid substitution—for example if the parental capsid is AAV9.AAA, then the liver tropism/detargeting may be relative to AAV9 without the peptide insert.

For example, as described in the Examples herein, the tissue tropism may be assessed by introducing an rAAV vector having the engineered capsid and a genome with a detectable transgene into a test animal, such as a mouse or NHP, for example by systemic, intravenous, intramuscular, intrathecal, subcutaneous, ocular, or other administration, at an appropriate dosage (for example 1E12, 1E13 or 1E14 vg/kg) and then after an appropriate period of time harvesting the tissues of the animal and assessing the presence of the vector genome, mRNA transcribed from the genome, the ratio of the mRNA to the vector DNA, transgene protein product or activity, including relative to the parental or reference capsid.

Accordingly, provided are capsids as described herein which, when administered (for example, IV, IM, subcutaneous administration) to an animal, including a mouse or NHP, exhibit at least 2-fold, 5-fold, 10-fold, 15 fold, 20-fold, or 25 fold, or greater tropism for muscle, including skeletal or heart, or other tissue such as retina or CNS tissue relative to a parental capsid or reference, such as AAV9, AAV9.AAA, AAVhu.32, or AAVhu.32.AAV, as measured by vector genome DNA, transgene mRNA, the ratio of mRNA to vector genome DNA, transgene protein product, including protein product activity. In embodiments, the capsid preferentially transduces neurons over astrocytes or other CNS tissue in the CNS of the animal. In embodiments, provided also are capsids as described herein which when administered (for example, IV, IM, subcutaneous administration) to an animal, including a mouse or NHP, exhibit at least 2-fold, 5-fold, 10-fold, 15 fold, 20-fold, 25 fold, 40-fold or 50-fold, less tropism for liver, either relative to a target tissue such as muscle (skeletal and/or heart), retina or CNS (including the ratio of target tissue to liver) and/or relative to a parental capsid or reference, such as AAV9, AAV9.AAA, AAVhu.32, or AAVhu.32.AAV, as measured by amount of vector genome DNA, transgene mRNA, the ratio of mRNA to vector genome DNA, transgene protein product, including protein product activity.

### 5.2.3 Additional AAV Capsid Insertion Sites

The following summarizes insertion sites for the peptides described herein, including the peptides in Tables 4, 5, 14, 15 and 16 set forth hereinbelow (see also, FIG. 1):
AAV1: 138; 262-272; 450-459; 595-593; and in an embodiment, between 453-454 (SEQ ID NO: 139).
AAV2: 138; 262-272; 449-458; 584-592; and in an embodiment, between 452-453 (SEQ ID NO: 140).
AAV3: 138; 262-272; 449-459; 585-593; and in an embodiment, between 452-453 (SEQ ID NO: 141).
AAV4: 137; 256-262; 443-453; 583-591; and in an embodiment, between 446-447 (SEQ ID NO: 142).
AAV5: 137; 252-262; 442-445; 574-582; and in an embodiment, between 445-446 (SEQ ID NO: 143).
AAV6: 138; 262-272; 450-459; 585-593; and in an embodiment, between 452-453 (SEQ ID NO: 144).
AAV7: 138; 263-273; 451-461; 586-594; and in an embodiment, between 453-454 (SEQ ID NO: 145).
AAV8: 138; 263-274; 451-461; 587-595; and in an embodiment, between 453-454 (SEQ ID NO: 146).
AAV9: 138; 262-273; 452-461; 585-593; and in an embodiment, between 454-455 (SEQ ID NO: 151).
AAV9.AAA: 138; 262-273; 452-461; 585-593; and in an embodiment, between 454-455 (SEQ ID NO: 158).
AAVrh10: 138; 263-274; 452-461; 587-595; and in an embodiment, between 454-455 (SEQ ID NO: 152).
AAVrh20: 138; 263-274; 452-461; 587-595; and in an embodiment, between 454-455 (SEQ ID NO: 155).
AAVrh74: 138; 263-274; 452-461; 587-595; and in an embodiment, between 454-455 (SEQ ID NO: 156 or SEQ ID NO: 157).
AAVhu.32: 138; 262-273; 452-461; 585-593; and in an embodiment, between 454-455 (SEQ ID NO: 148).
AAVhu.37: 138; 263-274; 452-461; 587-595; and in an embodiment, between 454-455 (SEQ ID NO: 153)

In embodiments, the peptide insertion occurs between amino acid residues 588-589 of the AAV9 capsid, or between corresponding residues of another AAV type capsid as determined by an amino acid sequence alignment (for example, as in FIG. 1). In embodiments, the peptide insertion occurs immediately after amino acid residue 1451 to L461, S268 and Q588 of the AAV9 capsid sequence, or immediately after corresponding residues of another AAV capsid sequence (FIG. 1).

In some embodiments, the capsid is chosen and/or further modified to reduce recognition of the AAV particles by the subject's immune system, such as avoiding pre-existing antibodies in the subject. In some embodiments. In some embodiments, the capsid is chosen and/or further modified to enhance desired tropism/targeting.

### 5.2.4 Generation of Modified Capsids

In some embodiments, AAV capsids were modified by introducing selected single to multiple amino acid substitutions which increase effective gene delivery to the CNS, detarget the liver, and/or reduce immune responses of neutralizing antibodies, prior to making the rAAV library with random peptide insertions.

Some modifications to the parental rAAV vector (capsid protein) prior to making the rAAV libraries may be effective for gene delivery to the CNS when intravenously administered rAAV vectors requires crossing the blood brain barrier. Key clusters of residues on the AAVrh.10 capsid that enabled transport across the brain vasculature and widespread neuronal transduction in mice have recently been reported. Specifically, AAVrh.10-derived amino acids N262, G263, T264, S265, G267, S268, T269, and T273 were identified as key residues that promote crossing the BBB (Albright et al, 2018, Mapping the Structural Determinants Required for AAVrh.10 Transport across the Blood-Brain Barrier). Amino acid substitutions in capsids, such as AAV8 and AAV9 capsids that promote rAAV crossing of the blood brain barrier, transduction, detargeting of the liver and/or reduction in immune responses have been identified.

In some embodiments, provided are capsids having one or more amino acid substitutions that further promote transduction and/or tissue tropism of the rAAV having the modified capsid. In embodiments, provided are capsids having a single mutation at amino acid 269 of the AAV8 capsid replacing alanine with serine (A269S) (see FIG. 1) and amino acid substitutions at corresponding positions in other AAV types. In some embodiments, provided are capsids having multiple substitutions at amino acids 263, 269, and 273 of the AAV9 capsid resulting in the following substitutions: S263G, S269T, and A273T see FIG. 1) or substitutions corresponding to these positions in other AAV types.

Exposure to the AAV capsid can generate an immune response of neutralizing antibodies. One approach to overcome this response is to map the AAV-specific neutralizing epitopes and rationally design an AAV capsid able to evade neutralization. A monoclonal antibody, specific for intact AAV9 capsids, with high neutralizing titer has recently been described (Giles et al, 2018, Mapping an Adeno-associated Virus 9-Specific Neutralizing Epitope To Develop Next-Generation Gene Delivery Vectors). The epitope was mapped to the 3-fold axis of symmetry on the capsid, specifically to residues 496-NNN-498 and 588-QAQAQT-592 of, e.g., AAV9, SEQ ID NO: 151. Capsid mutagenesis demonstrated that single amino acid substitution within this epitope markedly reduced binding and neutralization. In addition, in vivo studies showed that mutations in the epitope conferred a “liver-detargeting” phenotype to the mutant vectors, suggesting that the same residues are also responsible for AAV9 tropism. Liver detargeting has also been associated with substitution of amino acid 503 replacing tryptophan with arginine. Presence of the W503R mutation in the AAV9 capsid was associated with low glycan binding avidity (Shen et al, 2012, Glycan Binding Avidity Determines the Systemic Fate of Adeno-Associated Virus Type 9).

In some embodiments, provided are capsids which were further modified by substituting asparagines at amino acid positions 498, 499, and 500 (such as AAV8.AAA) or 496, 497, and 498 (herein referred to as AAV9.AAA, SEQ ID NO: 158) with alanines. In some embodiments, the AAVrh10 capsid can also be modified by substituting three asparagines at amino acid positions 498, 499, and 500 to alanines (AAVrh10.AAA).). In some embodiments, the AAVhu32 capsid can also be modified by substituting three asparagines at amino acid positions 496, 497, and 498 to alanines (herein referred to as AAVhu.32.AAA, SEQ ID NO: 148).

In some embodiments, provided are capsids having three asparagines at amino acid positions 496, 497, and 498 of the AAV9 capsid replaced with alanines and also tryptophan at amino acid 503 of the AAV9 capsid with arginine or capsids with substitutions corresponding to these positions in other AAV types. In some embodiments, provided are capsids having glutamine at amino acid position 474 of the AAV9 capsid substituted with alanine or capsids with substitutions corresponding to this position in other AAV types.

In some embodiments, the rAAVs described herein increase tissue-specific (such as, but not limited to, muscle) cell transduction in a subject (a human, non-human-primate, or mouse subject) or in cell culture, compared to the rAAV not comprising the peptide insertion. In some embodiments, the increase in tissue specific cell transduction is at least 2, 10, 20, 30, 40, 50, 60, 70, 80, 90, or 100 fold more than that without the peptide insertion. For example, in some embodiments, there is a 50-80 fold increase in tissue specific cell transduction compared to transduction with the same AAV type without a peptide insert. The increase in transduction may be assessed using methods described in the Examples herein and known in the art.

### 5.3. Methods of Making rAAV Molecules

Another aspect of the present invention involves making molecules disclosed herein. In some embodiments, a molecule according to the invention is made by providing a nucleotide comprising the nucleic acid sequence encoding any of the capsid protein molecules herein; and using a packaging cell system to prepare corresponding rAAV particles with capsid coats made up of the capsid protein. In some embodiments, the nucleic acid sequence encodes a sequence having at least 60%, 70%, 80%, 85%, 90%, 91%, 92%, 93%, 94%, 95%, 96%, 97%, 98%, 99% or 99.9%, identity to the sequence of a capsid protein molecule described herein, and retains (or substantially retains) biological function of the capsid protein and the inserted peptide from a heterologous protein or domain thereof. In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the AAV9 capsid protein (SEQ ID NO: 151 and see FIG. 1), while retaining (or substantially retaining) biological function of the AAV9 capsid protein and the inserted peptide.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In other embodiments, the cap and rep genes are provided by a packaging cell and not present in the viral genome.

In some embodiments, the nucleic acid encoding the engineered capsid protein is cloned into an AAV Rep-Cap helper plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In some embodiments, the rAAVs provide transgene delivery vectors that can be used in therapeutic and prophylactic applications, as discussed in more detail below. In some embodiments, the rAAV vector also includes regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue. In specific embodiments, the AAV vector comprises a regulatory sequence, such as a promoter, operably linked to the transgene that allows for expression in target tissues. The promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter, opsin promoter, the TBG (Thyroxine-binding Globulin) promoter, the APOA2 promoter, SERPINA1 (hAAT) promoter, or MIR122 promoter. In some embodiments, particularly where it may be desirable to turn off transgene expression, an inducible promoter is used, e.g., hypoxia-inducible or rapamycin-inducible promoter.

Provided in embodiments are AAV9 vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV9 capsid protein (see FIG. 1), while retaining the biological function of the engineered AAV9 capsid. In certain embodiments, the encoded AAV9 capsid has the sequence of wild type AAV9, with the peptide insertion as described herein, with, in addition, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions with respect to the wild type AAV sequence and retains biological function of the AAV9 capsid. Also provided are engineered AAV vectors other than AAV9 vectors, such as engineered AAV1, AAV2, AAV3, AAV4, AAV5, AAV6, AAV7, AAV8, AAV9, AAV9e, AAVrh10, AAVrh20, AAVhu.37, AAVrh39, or AAVrh74 vectors, with the peptide insert as described herein and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to the wild type or unengineered sequence for that AAV type and that retains its biological function.

The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety

The rAAV vector for delivering the transgene to target tissues, cells, or organs, has a tropism for that particular target tissue, cell, or organ. Tissue-specific promoters may also be used. The construct further can include expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV9 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) transgene providing (e.g., coding for) a nucleic acid or protein product of interest. In a specific embodiment, the constructs described herein comprise the following components: (1) AAV9 inverted terminal repeats that flank the expression cassette; (2) control elements, which include a) a hypoxia-inducible promoter, b) a chicken β-actin intron and c) a rabbit β-globin poly A signal; and (3) transgene providing (e.g., coding for) a nucleic acid or protein product of interest.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or other cell types may be used, for example, but not limited, to HuH-7, HEK293, fibrosarcoma HT-1080, HK3-11, and CAP cells. Once expressed, characteristics of the expressed product (i.e., transgene product) can be determined, including determination of the glycosylation and tyrosine sulfation patterns, using assays known in the art.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 1A-1B below provides an exemplary list of transgenes that may be used in any of the rAAV vectors described herein, in particular, to treat or prevent muscle-related disease.

TABLE 1A

| Disease | Transgene |
|---|---|
| Spinal Muscular Atrophy (SMA) | SMN |
| Duchenne Muscular Dystrophy | Mini-/Microdystrophin Gene |
| Limb Girdle Muscular Dystrophy Type | human-alpha-sarcoglycan |
| 2C\|Gamma-sarcoglycanopathy | |
| Limb Girdle Muscular Dystrophy Type | gamma-sarcoglycan |
| 2C\|Gamma-sarcoglycanopathy | |
| Becker Muscular Dystrophy and Sporadic | huFollistatin344 |
| Inclusion Body Myositis | |
| Duchenne Muscular Dystrophy | GALGT2 |
| Limb-Girdle Muscular Dystrophy, Type 2E | hSGCB |

TABLE 1B

| ANTIGENS | | TRANSGENE/ANTIBODIES | INDICATIONS |
|---|---|---|---|
| Repulsive guidance molecule-A | | elezanumab | multiple sclerosis |
| Complement Component 5 | | ravulizumab | Myasthenia Gravis |
| Connective tissue growth factor | | pamrevlumab | fibrotic diseases, e.g. |
| (CTGF) | | | diabetic nephropathy, |
| | | | liver fibrosis, idiopathic |
| | | | pulmonary fibrosis |
| Integrin beta 7 | | etrolizumab | ulcerative colitis, |
| | | | Crohn's disease |
| Sclerostin | | romosozumab | Osteoporosis, |
| | | (EVENITY ®) | abnormal bone loss or |
| | | | weakness |
| Interleukin receptor 6 (IL6R) or | | Satralizumab | Adverse immune |
| Interleukin 6 (IL6) | | Sarilumab | responses (e.g. cytokine |
| | | Tocilizumab | storm, CAR-T therapy) |
| | | siltuximab, | |
| | | clazakizumab | |
| | | sirukumab | |
| | | olokizumab | |
| | | gerilimzumab | |
| Immunoglobin E (IgE) | | omolizumab | Asthma, COPD, |
| | | | eosinophilic asthma, |
| | | | chronic idiopathic |
| | | | urticaria |
| Thymic stromal lymphopoietin | | tezelipumab | Asthma, COPD |
| (TSLP) | | | |
| Interleukin 5 (IL5) | | benralizumab | Asthma, COPD |
| Interleukin 5 receptor (IL5R) | | reslizumab | Asthma, COPD, |
| | | | eosinophilic asthma |
| Interleukin 13 (IL13) | | tralokinumab | Atopic dermatitis |
| Interleukin 31 recptor alpha | | nemolizumab | Atopic dermatitis |
| (IL31RA) | | | |
| IL17A | | Ixekizumab | Plaque psoriasis, |
| | | secukinumab | psoriatic arthritis, |
| | | | ankylosing sponylitis |
| Interleukins or | IL-17A | ixekizumab (TALTZ ®) | Plaque psoriasis, |
| | | secukinumab | psoriatic arthritis, |
| | | (COSENTYX ®) | ankylosing sponylitis |
| interleukin | IL-5 | mepolizumab | Asthma |
| | | (NUCALA ®) | |
| receptors | IL-12/IL-23 | ustekinumab | Psoriasis & Crohn's |
| | | (STELARA ®) | disease |
| | IL-4R | dupilumab | Atopic dermatitis |
| Integrin | | vedolizumab | Ulcerative colitis & |
| | | (ENTYVIO ®) | Crohn's disease |
| | | Natalizumab (anti-integrin | Multiple sclerosis & |
| | | alpha 4) | Crohn's disease |
| Cardiovascular | PCSK9 | alirocumab | HeFH & HoFH |
| Targets | | (PRALUENT ®) | |
| | | evolucomab (REPATHA ®) | |
| | ANGPTL3 | evinacumab | HoFH & severe forms |
| | | | of dyslipidema |
| | Proinflammatory/ | E06-scFv | Cardiovascular diseases |
| | proatherogenic | | such as atherosclerosis |
| | phospholipids | | |
| RANKL | | denosumab (XGEVA ® and | Osteoporosis, |
| | | PROLIA ® | increasing bone mass in |
| | | | breast and prostate |
| | | | cancer patients, & |
| | | | preventing skeletal- |
| | | | related events due to |
| | | | bone metastasis |
| PD-1, or PD-L1 or PD-L2 | | nivolumab (OPDIVO ® | Metastatic melanoma, |
| | | pembrolizumab | lymphoma, non-small |
| | | (KEYTRUDA ®) | cell lung carcinoma |
| BLyS (B-lymphocyte stimulator, | | belimumab | Systemic lupus |
| also known as B-cell activating | | (BENLYSTA ®) | erythromatosis |
| factor (BAFF)) | | | |
| TNF-alpha | | adalimumab (HUMIRA ®) | Rheumatoid arthritis, |
| | | and | psoriatic arthritis, |
| | | infliximab (REMICADE ®) | askylosing spondylitis, |
| | | | Crohn's disease, plaque |
| | | | psoriasis, ulcerative |
| | | | colitis |
| Plasma Protein | C5, C5a | eculizumab (SOLIRIS ®) | Paroxysmal nocturnal |
| targets | | | hemoglobinuria, |
| | | | atypical hemolytic |
| | | | uremic syndrome, |
| | | | complement-mediated |
| | | | thrombotic |
| | | | microangiopathy |
| | Plasma kallikrein | lanadelumab | Hereditary angioedema |
| | | | (HAE) |

For example, a rAAV vector comprising a transgene encoding a microdystrophin finds use treating/preventing/managing Duchenne Muscular Dystrophy. The microdystrophin may be, for example, a microdystrophin found in WO 2021/108755. In other embodiments, the microdystrophin has an amino acid sequence of SEQ ID NO: 133 (human MD1 (R4-R23/ΔCT), SEQ ID NO: 134 (microdystrophin), SEQ ID NO: 135 (Dys3978), SEQ ID NO: 136 (MD3) or SEQ ID NO: 137 (MD4) as described in WO 2023/004331. In other embodiments, the microdystrophin is SEQ ID NO: 7 of WO 2017/181015 A1. Generally, the rAAV vector is administered systemically. For example, the rAAV vector may be provided by intravenous, intramuscular, intra-nasal, and/or intra-peritoneal administration.

In aspects, the rAAVs of the present invention find use in delivery to target tissues, or target cell types, including cell matrix associated with the target cell types, associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject tan rAAV where the peptide insertion is a homing peptide. In the case of dystrophinopathies, for example, a rAAV vector comprising a peptide insertion that directs the rAAV to muscle tissue can be used, in particular, where the peptide insertion facilitates the rAAV in transducing muscle cells with high efficiency, including satellite cells, yet results in lower transduction of liver cells.

For a disease or disorder associated with muscle, rAAV vectors can be selected from the libraries herein that comprise a peptide insertion that directs muscle transduction, relative to the parental rAAV vector without a peptide insertion.

The rAAV vectors of the invention also can facilitate delivery, in particular, targeted delivery, of oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. Also, the rAAV molecule of the invention may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising a dual antigen-binding molecule of the invention, may increase or decrease over the course of treatment.

In some embodiments, ongoing treatment is indicated, e.g., on a long-term basis, such as in the ongoing treatment and/or management of chronic diseases or disorders. For example, in embodiments, an agent of the invention is administered over a period of time, e.g., for at least 6 months, at least one year, at least two years, at least five years, at least ten years, at least fifteen years, at least twenty years, or for the rest of the lifetime of a subject in need thereof.

The rAAV molecules of the invention may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In embodiments, such as where the transgene is intended to be expressed in the CNS, the vector is administered via lumbar puncture or via cisterna magna.

In certain embodiments, the agents of the invention are administered intravenously and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref Biomed. Eng., 14:20, 1987; Buchwald et al., Surgery, 88:507, 1980; and Saudek et al., N. Engl. J Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, rAAVs can be used for in vivo delivery of transgenes for scientific studies such as optogenetics, gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In certain embodiments, the agent of the invention is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

The following examples report an method of making rAAV libraries containing numerous rAAV capsids having surface-exposed peptides inserted at designated insertion sites of the capsid. The recombinantly engineered capsids are screened to identify candidates for particularly properties, such as tissue tropism. The invention is illustrated by way of examples, describing the construction of rAAV capsids engineered to contain 7-mer peptides (or 4-, 5-, 6-mer peptides), wherein the method is designed to remove bias of the parental capsid formation during manufacture, thereby artificially increasing its abundance and therefore representation in the library. Several libraries of peptide insertion mutants were constructed and the pooled mutants were screened for viable capsid assembly, titer, and biodistribution. The top candidates were then further evaluated for use to re-target rAAV vectors to tissues of interest. Further examples, demonstrate the increased transduction and tissue tropism for certain of the modified AAV capsids described herein.

### 6.1. Example 1—Insertion Sites of AAV9 Capsid or Capsids Having Corresponding Sites For Insertion

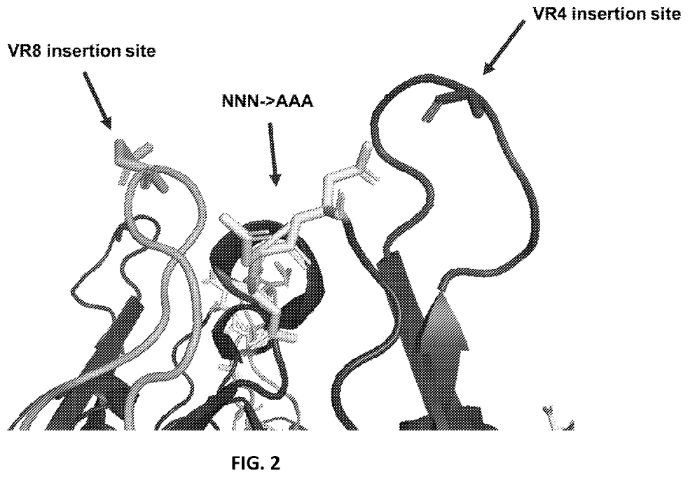

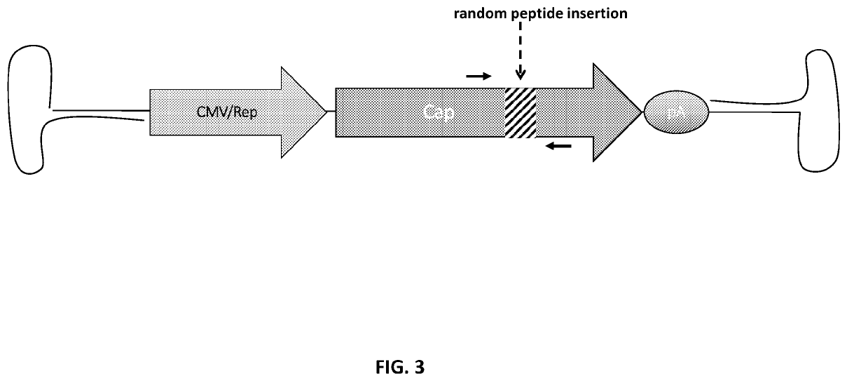

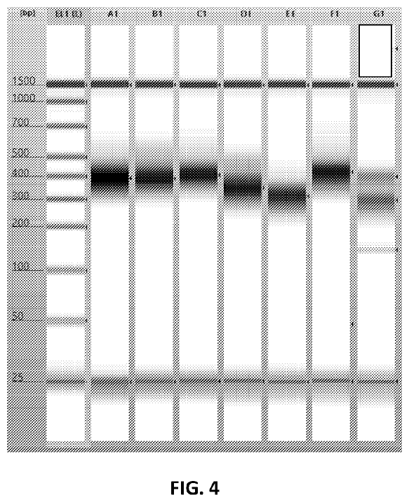

FIGS. 1 and 2 depict analysis of variable region four of the adeno-associated virus type 9 (AAV9 VR-IV) by amino acid sequence comparison to other AAVs VR-IV (FIG. 1) and protein model (FIG. 2). As seen, AAV9 VR-IV is exposed on the surface at the tip or outer surface of the 3-fold spike. AAV9 VR-VIII is also exposed on the surface. Further analysis has indicated that there are polar interactions between VR-TV and VR-V and that the sequence and structure of VR-IV is variable amongst AAV serotypes, and further that there is potential for interrupting a commonly-targeted neutralizing antibody epitope and thus, reducing immunogenicity of the modified capsid. (See PCT International Publication No. WO2020206189A1, which is hereby incorporated by reference in its entirety.)

### 6.2. Example 2—Construction of rAAV Libraries

For generation of rAAV libraries, a custom Rep-only trans-plasmid was created in which the Cap sequence was removed. A custom cis-plasmid was created in which the CMV enhancer-promoter and Rep intron precedes the AAV Cap gene of interest, followed by the RBG polyA. A stop codon was inserted into the Cap gene variable region where the peptide library would be later inserted to reduce expression of wildtype (or parental) sequence in the library and provide capsids capable of “intelligent, guided adaptation” (Novel AAV Vector Intelligent Guided Adaptation Through Evolution=NAVIGATE.)

AAV peptide insertion libraries were created by synthesis of a DNA fragment incorporating a randomized 21 nucleotide sequence synthesized using column-based Trimer-19 oligos (Genscript). The library fragment was cloned into the desired AAV variable region in the library cis-plasmid, such that the stop codon inserted in the parental capsid of interest would be removed. Following transformation of E. coli, library size was quantified by dilution plate colony counts. Library size generally exceeded 10\hat{ }7 variants. Next-generation sequencing of the libraries was used to characterize library diversity and the wildtype fraction. A plasmid maxiprep was used to generate 0.5-1 mg of library cis-plasmid.

To produce the library vector, a custom triple transfection of suspension-adapted HEK293 cells (NAVXCell™, REGENXBIO Inc.) was used in which the library cis-plasmid was limited to 100-copies per viable cell (typically ˜3,000) to reduce the likelihood of variant cross-packaging and capsid mosaicism. A 20L- or 8L-scale transfection was used to generate libraries. Three days post-transfection, cells were harvested, lysed, clarified, PEG-precipitated, and purified using Iodixanol-gradient ultracentrifugation.

Vector titer was quantified using digital PCR with polyA primers/probe. Library variant diversity and parental/wildtype fraction following AAV packaging was characterized by next-generation sequencing (NGS) and LC-MS of the VP3 protein. Several libraries were made starting from different parental capsids, and evaluation indicated a high level of diversity in each capsid library. See, e.g. Table 2.

Each library was produced at 20L scale, as described above, by the vector core group at REGENXBIO Inc. Library diversity and titer measurements determined that diversity ranged from 1E7-1E8 in such production lots having final BDS titer ranging from 1E12-1E13 (Table 2). Using deep NGS techniques (NovaSeq™, Illumina) to understand the input variant distribution, NGS testing obtained approximately 300 million reads per library.

TABLE 2

| | | BDS Titer | |
|---|---|---|---|
| Lane | Library | (GC/mL) | Library diversity |
| A1 | AAV8.VR4 | 3.51E+13 | 4.32E+07 |
| B1 | AAV8.VR8 | 8.15E+13 | 1.15E+07 |
| C1 | AAV9.VR4 | 1.50E+13 | 1.02E+07 |
| D1 | AAV9.VR8 | 4.55E+12 | 1.45E+07 |
| E1 | AAV4.VR4 | 4.25E12 | 2.26E+07 |
| F1 | AAV3B.VR4 | 2.84E+12 | 2.70E+08 |
| G1 | AAV5.VR8 | 3.97E+13 | 1.36E+07 |

NGS analysis of initial vector libraries had high parental vector levels (FIG. 5A), as indicated by the larger representation of parental vector following production, compared to the percent parental plasmid in the initial preparation of the library. However, the stop codon inserted into the template plasmid prevented wildtype (parental) vector production from carryover template present in the production culture after library cloning. The drop-in vector stop codon abundance levels indicate selection against cross-packaging (FIG. 5B).

Parental fraction following AAV packaging was further characterized by LC-MS of the VP3 protein. Following generation of an AAV5 vector library with no stop codon in template, the parental (AAV5) VP3 theoretical wildtype mass is 59,551.64 Da and AAV5 VP3 with peptide insertion is 59,951 to 60,855 Da. FIG. 6A and FIG. 6B illustrate (by LC-MS analysis of VP3 proteins in each version of the library) that the addition of stop codon (FIG. 6B) significantly reduces the packaging of parental vector.

### 6.3. Example 3—Evaluation and Selection of Capsids from Various Libraries

Samples of vector libraries that were made according to the above methods are listed in Table 3 and were dosed accordingly to non-human primates (NHPs; Cynomolgous macaques) of approximately 2-3 years at study start date. As such, three NHPs per study were dosed at 9.3e10 to 2.9e13 GC/kg by different routes of administration under ketamine sedation, as indicated in Table 3.

TABLE 3

| | Round 1 | Insertion | | |
|---|---|---|---|---|
| Library # | Library | Loop | ROA | Dosc |
| 1 | AAV5 | VR8 | IV | 2.9E13 GC/kg |
| 2 | AAV4 | VR4 | | 9.30E10 |
| | | | | GC/kg |
| 3 | AAV8 | VR4 | | 9.7E12 GC/kg |
| | | | SCS | 2.11E12 |
| | | | | GC/eye |
| 4 | AAV8 | VR8 | IV | 1.8E13 GC/kg |
| | | | SCS | 9.8E11 GC/eye |
| 5 | AAV9.NNN | VR4 | IV | 1.0E13 GC/kg |
| 6 | AAV9.NNN | VR8 | | 2.2E12 GC/kg |
| 7 | AAV3B | VR4 | | 6.7E11 GC/kg |
| | | | IVT | 1.42E11 |
| | | | | GC/eye |
| 8 | AAV1 | VR4 | ICV | |
| 9 | AAVhu37 | VR4 | | |

Blood was collected immediately prior to dosing, and serum collected and processed. Twenty-one (21) days following dosing, animals were euthanized and blood and target tissues were collected. Tissues were examined, weighed, and then placed into RNAse/DNase-free cryovials and flash frozen in liquid nitrogen. Based on the size of the specified tissue/region, up to five samples of approximately 50-100 mg per sample were collected from each location. Tissue samples were collected using aseptic technique and RNAse-free instruments and workspaces, with care taken to ensure no cross contamination between tissues. Tissues were flash frozen in liquid nitrogen and then maintained on dry ice prior to storage at −70 to −90° C. DNA and RNA was extracted from all tissues by standard techniques and next generation sequencing (NGS) and quantitative PCR (mRNA transcript expression) was performed, respectively. A custom bioinformatics platform was employed utilizing open-source software packages and R-scripts to analyze cDNA counts and RNA-seq data. For example, merge_counts.R was utilized to merge peptide counts from all samples into a single dataframe, based on methods well-known in the art and available on github.com. Analysis of the vector repertoire in multiple tissues revealed that 1795 peptides in the AAV9.AAA.VR4 library were detected in one or more muscle tissue samples. The pool of top hits from this library were further evaluated in a similar NHP study.

### 6.4. Example 4—Evaluation and Selection of Capsids Transducing Muscle from an AAV9.AAA Library

The 496NNN498→AAA mutation in AAV9 (SEQ ID NO: 151) provides greater than 100-fold reduced liver transduction. The AAV9.AAA parental capsid was used as a starting template for producing a vector library having random peptide insertions after amino acid residue S454, to generate a AAV9.AAA.VR4 vector library, using the methods described in the Example hereinabove.

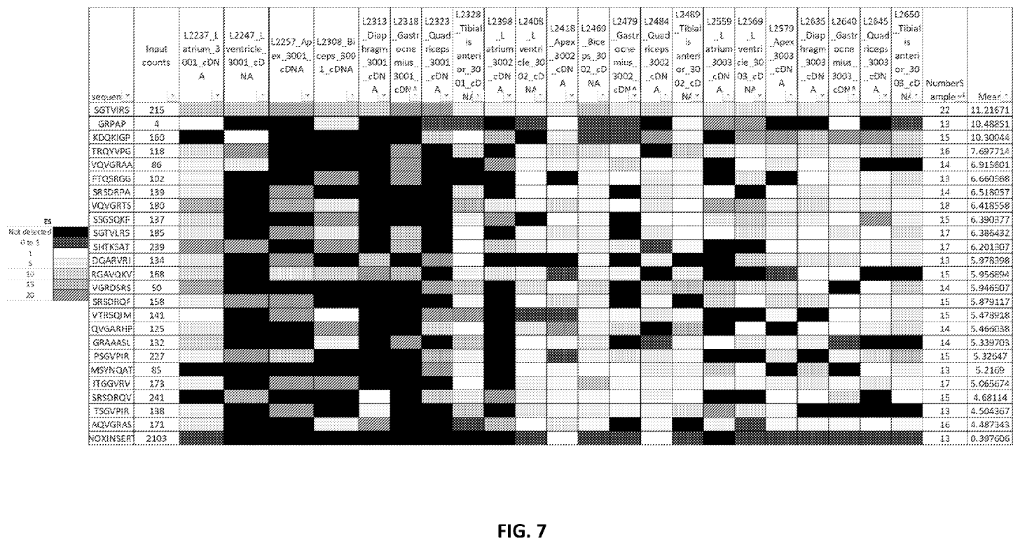

Following the analysis of a vector repertoire containing a diverse set of peptides having tropism for one or more muscle tissue samples in the first round of animal testing, a pool from AAV9.AAA.VR4 was further analyzed by dosing NHPs at 1.52e13 GC/kg. The bioinformatics tools were used to determine vector performance ranking based on readouts such as relative abundance (RA), ES counts, etc. (e.g. using Dunnett's multiple comparison test p-values) to determine an enrichment score. A representative list of 134 hits (peptides) and 4 consensus sequences emerged, as in Tables 4, 5, 14, 15 and 16 for example, based on selection in muscle, compared to the parental spiked-in vector control (AAV9.AAA). Top 24 hits (peptides) analyzed by NGS in various tissues are represented in FIG. 7.

TABLE 4

| SEQ | |
|---|---|
| ID NO: | Peptide sequence |
| 1 | SGTVIRS |
| 2 | GRPAP |
| 3 | KDQKIGP |
| 4 | TRQYVPG |
| 5 | VQVGRAA |
| 6 | FTQSRGG |
| 7 | SRSDRPA |
| 8 | VQVGRTS |
| 9 | SSGSQKF |
| 10 | SGTVLRS |
| 11 | SHTKSAT |
| 12 | DQARVRI |
| 13 | RGAVQKV |
| 14 | VGRDSRS |
| 15 | SRSDRQF |
| 16 | VTRSQIM |
| 17 | QVGARHP |
| 18 | GRAAASL |
| 19 | PSGVPIR |
| 20 | MSYNQAT |
| 21 | ITGGVRV |
| 22 | SRSDRQV |
| 23 | TSGVPIR |
| 24 | AQVGRAS |
| 25 | VKASHHS |
| 26 | SHTKTVT |
| 27 | GSVQTVR |
| 28 | DTRIRSM |
| 29 | SRTKTAT |
| 30 | QVGARQS |
| 31 | PSGQRLH |
| 32 | GQVHQAR |
| 33 | TSGVQIR |
| 34 | SIAKTSS |
| 35 | SRADRHV |
| 36 | VQVGRVS |
| 37 | SVTSVRV |
| 38 | TSGGQIR |
| 39 | SHIKTAT |
| 40 | SHIKTST |
| 41 | GNIKYGS |
| 42 | SRSDRHV |
| 43 | TSGAPIR |
| 44 | VQVGRSS |
| 45 | MKYRGTD |
| 46 | HFAGKSV |
| 47 | IKYRGTD |
| 48 | SIAKHSS |
| 49 | SHTRTAT |
| 50 | SGTGIRA |
| 51 | SIAKHGA |
| 52 | GRAAATL |
| 53 | SGTVLRA |
| 54 | ASAKHAA |
| 55 | AHTKTAT |
| 56 | VKASHAS |
| 57 | SNTKHAT |
| 58 | VPVGRAS |
| 59 | SISKHSA |
| 60 | QKGRNPD |
| 61 | QSAVRHP |
| 62 | VQGGRAS |
| 63 | VKNSSGTF |
| 64 | HFTHQSK |
| 65 | DKRGVIV |
| 66 | HMGQSGK |
| 67 | GQVHQSR |
| 68 | SHTKPAT |
| 69 | KDSGGYR |
| 70 | RRVSNVD |
| 71 | ARVTASI |
| 72 | GNIKSGS |
| 73 | DKRGVIG |
| 74 | SIAKTAA |
| 75 | QSVGRYS |
| 76 | SIAKNSA |
| 77 | VSGAVRVT |
| 78 | PSSKARQ |
| 79 | MARPQSI |
| 80 | TVRGREQ |
| 81 | SHTRTST |
| 82 | HMGQYGK |
| 83 | TVATSKI |
| 84 | RAVTLGV |
| 85 | FHTKTAT |
| 86 | QQAQR |
| 87 | SIARHSA |
| 88 | HMGHSGK |
| 89 | ATRGVIG |
| 90 | SKTFAAS |
| 91 | VVAGAMR |
| 92 | SSGAQKF |
| 93 | EMTSRHV |
| 94 | RRVSTVD |
| 95 | ITGEVRG |
| 96 | IQGVHSK |
| 97 | SPARA |
| 98 | AGEGP |
| 99 | AGPGA |
| 100 | RQARR |
| 101 | GNIQSRS |
| 102 | IIHNQAK |
| 103 | PKNVKTT |
| 104 | DMNSRHV |
| 105 | ENRGIIG |
| 106 | YTKTSSV |
| 107 | LVNQQKS |
| 108 | PSSKDRH |

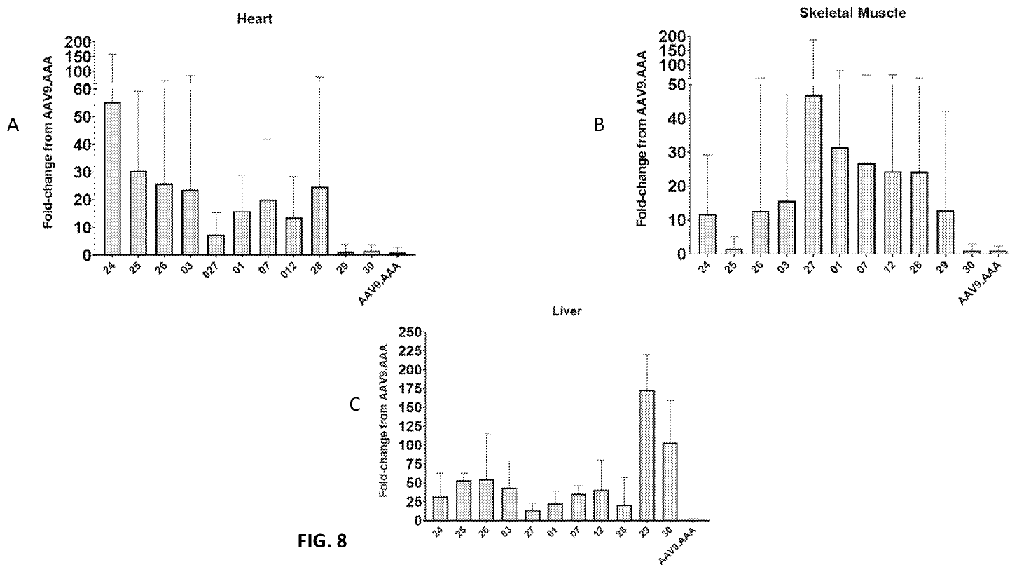

Skeletal muscle and heart tissue data was compared to liver as in FIG. 8A-C, represented as fold change RA relative to the control parental vector. Vector comprising capsid having peptide 01 (SEQ ID NO: 1) exhibited 32-fold increase in relative abundance (mRNA expression) in skeletal muscle and 16-fold in heart. Peptide 24 (SEQ ID NO: 24) insertion resulted in vector that exhibited 55-fold in heart and 12-fold in skeletal muscle. Whereas vectors having insertion of peptide 29 (SEQ ID NO: 29) or peptide 30 (SEQ ID NO: 29) showed increased levels in liver 173-fold and 103-fold, respectively, relative to the parental capsid.

Following the analysis of a vector repertoire in the second round of animal testing, a third pool (see Table 5) of AAV9.AAA.VR4 capsids as well as capsids from the AAV5.VR8 library (including peptides SEQ ID NO: 118, SEQ ID NO: 119 and SEQ ID NO: 120 in AAV5) was further analyzed in NHP. All study animals were screened for the presence of AAV2, AAV8 and AAV9 neutralizing antibodies in serum by in vitro neutralizing antibody assay (Precision for Medicine) and only animals with titers below a defined threshold were enrolled in the study. Pre-dose blood collections were taken (following ketamine sedation) and placed into serum separator tubes. After a minimum 15 minutes at ambient temperature, blood was centrifuged (2500×g for 10 minutes) and serum harvested.

TABLE 5
Peptides analyzed in Round 3

| Designation | SEQ ID NO: | Sequence | AAV Capsid |
|---|---|---|---|
| NVG01 | SEQ ID NO: 32 | GQVHQAR | AAV9.AAA.VR4 |
| NVG02 | SEQ ID NO: 38 | TSGGQIR | AAV9.AAA.VR4 |
| NVG03 | SEQ ID NO: 109 | MRAVSQL | AAV9.AAA.VR4 |
| NVG04 | SEQ ID NO: 4 | TRQYVPG | AAV9.AAA.VR4 |
| NVG05 | SEQ ID NO: 88 | HMGHSGK | AAV9.AAA.VR4 |
| NVG06 | SEQ ID NO: 1 | SGTVIRS | AAV9.AAA.VR4 |
| NVG07 | SEQ ID NO: 8 | VQVGRTS | AAV9.AAA.VR4 |
| NVG08 | SEQ ID NO: 13 | RGAVQKV | AAV9.AAA.VR4 |
| NVG09 | SEQ ID NO: 110 | PRQYVPG | AAV9.AAA.VR4 |
| NVG10 | SEQ ID NO: 111 | RSSSGR | AAV9.AAA.VR4 |
| NVG11 | SEQ ID NO: 112 | VVKSTKS | AAV9.AAA.VR4 |
| NVG12 | SEQ ID NO: 113 | RHVSASD | AAV5.(578)VR8 |
| NVG13 | SEQ ID NO: 114 | VRSDRDQ | AAV5.(578)VR8 |
| NVG14 | SEQ ID NO: 115 | TVVTSIN | AAV5.(578)VR8 |

Test article (pooled capsids) were administered IV (2e13 GC/kg) to each of 3 animals. Test article were administered by IV bolus under ketamine sedation (ketamine hydrochloride (HCl) delivered by intramuscular (IM) injection) followed by a flush of at least 5 mL saline. Following 21 days, animals were anesthetized with ketamine HCl delivered by IM injection. Blood was collected and placed into serum separator tubes. After a minimum 15 minutes at ambient temperature, blood was centrifuged (2500×g for 10 minutes) and serum harvested. Collected tissues were examined, weighed, and then placed into RNAse/DNase-free cryovials. Based on the size of the specified tissue/region, up to five samples of approximately 50-100 mg per sample were collected from each location. Tissue samples were collected using aseptic technique and RNAse-free instruments and workspaces. Tissues were flash frozen in liquid nitrogen and then maintained on dry ice prior to storage at −70 to −90° C.

DNA (Kingfisher DNA) and mRNA (Dynabeads mRNA direct) were extracted from the tissues and the vector barcode sequences were amplified by PCR and then detected by NGS on the Illumina Miseq. Units of “nRAAFI”, normalized relative abundance adjusted for input, were calculated. Essentially, the relative abundance of each barcode was divided by the input relative abundance and then normalized to a sum of one in each sample to identify the nRAAFI for each barcoded transgene.

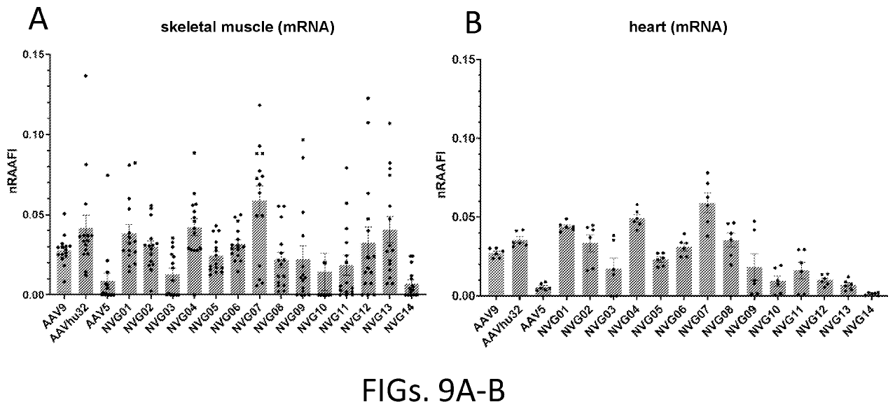

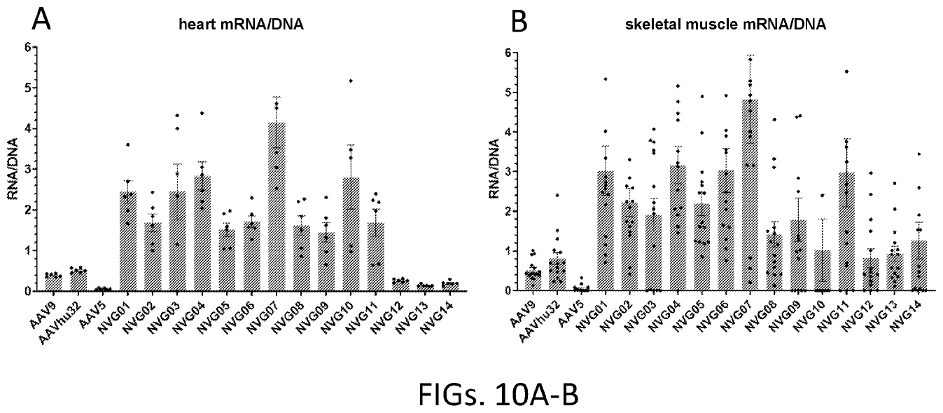

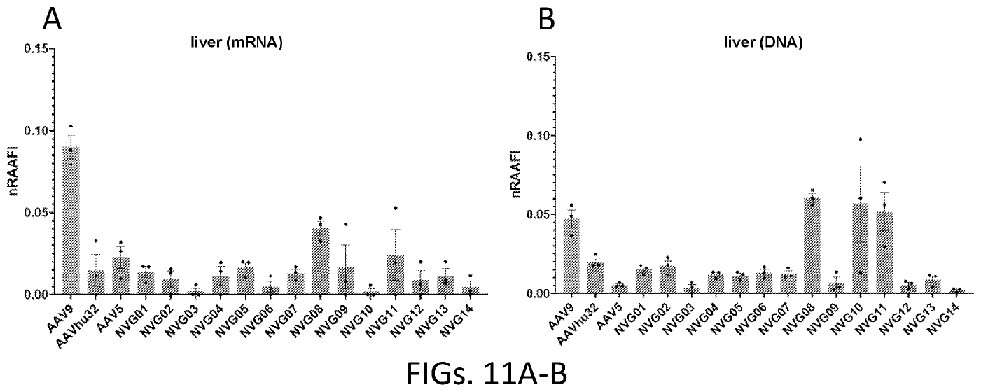

FIG. 9 shows that AAV9.AAA.NVG07 mediated a 2-fold increase in mRNA expression in skeletal muscle (FIG. 9A) and heart (FIG. 9B) compared with AAV9. FIG. 10 shows that AAV9.AAA.NVG07 provided a 10 fold greater ratio of mRNA/DNA in heart (FIG. 10A) and skeletal muscle (FIG. 10B) compared with AAV9. FIG. 11 shows the levels of transgene mRNA (A) compared to DNA (B) in liver. AAV9.AAA.NVG07 is greater than 7 fold liver de-targeted in mRNA compared to AAV9. Several capsids with peptide insertions (from the AAV9.AAA.VR4 or AAV5.VR8 vector library) displayed a favorable biodistribution with respect to muscle vs. liver transduction, some capsids even displaying lower heart transduction which may be desirable for certain transgenes.

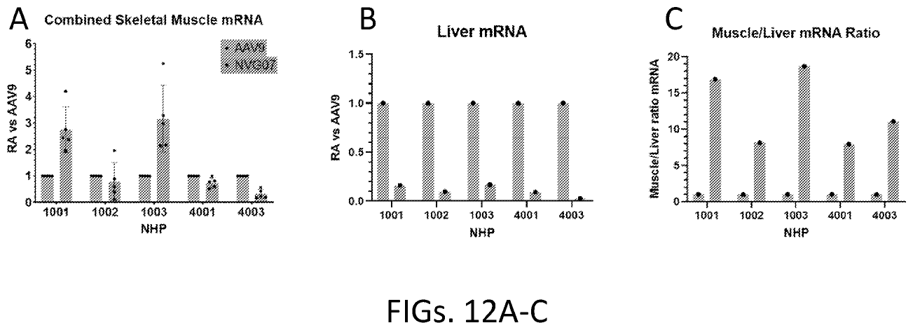

The data from the present NHP study for NVG07 mRNA distribution in skeletal muscle and liver was selected and combined with the NVG07 data from animals 4001 and 4003 from the study described in Example 6, below, that tested the same capsids. AAV9 data values were also taken from these NHP studies and were normalized to 1 to show the fold difference of NVG07 compared to AAV9. FIG. 12A (Skeletal muscle) and FIG. 12B (liver). The ratio of muscle mRNA to liver mRNA was also calculated for each animal. As shown in FIG. 12C, the NVG07 transduction of muscle tissue over liver tissue is approx. 8 to 18 times better than AAV9.

### 6.5. Example 5: Assessment of Selected Capsids In Vitro and In Vivo

AAV capsid sequences modified by peptide insertions with or without additional substitutions are further evaluated in an in vitro assay, as well as for in vivo bio-distribution in test animals using next generation sequencing (NGS) and quantitative PCR.

Studies are performed where selected vectors from the library are produced by method described in these Examples, or by standard methods of triple transfection utilizing Rep/cap trans plasmid (containing the engineered capsid gene sequence), cis plasmid encoding a transgene, and helper gene plasmid. The engineered vectors of interest are injected individually into test animals, e.g. NHPs, mice or rats, and DNA and RNA determinations from extracted tissues are compared to the parental vectors. Quantitative PCR (qPCR) can be done on a QuantStudio 5 (Life Technologies, Inc.) or any standard method such as ddPCR, using primer-probe combinations specific for the transgene.

From the studies where individual vectors are injected into test animals for characterization, formalin fixed brains may be, e.g. sectioned at 40 μm thickness on a vibrating blade microtome (VT1000S, Leica) and the floating sections probed with antibodies against transgene (or viewed for fluorescence if fluorescent transgene) to look at the cellular distribution of the delivered vectors.

C57B16 mice (n=5 per test article group) were administered AAV.hu32, AAV9.AAA.VQVGRTS (NVG07) or AAVhu.32.AAA.VQVGRTS capsids carrying a CAG.TdTomato transgene, 1E14 GC/kg IV (tail vein) administration per animal. Following 3 weeks, animals were sacrificed and heart, gastrocnemius, quadriceps, bicep, brain, liver and diaphragm tissues were collected.

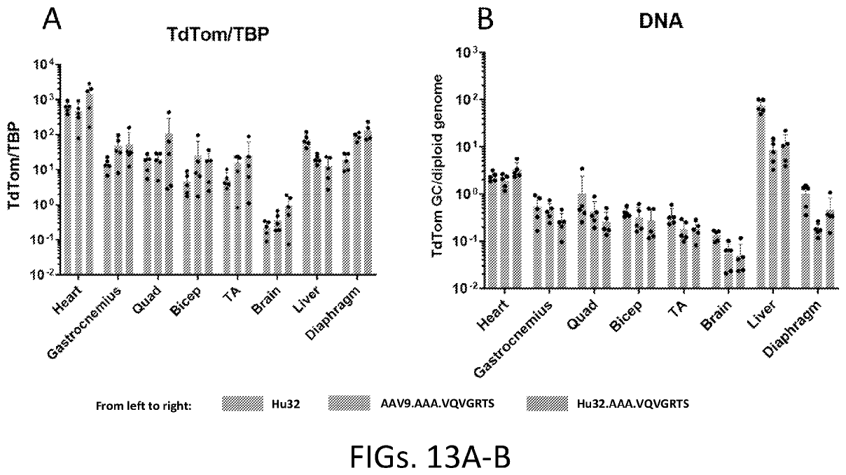

TdTomato genome (cDNA) and transcripts (mRNA) were detected by digital PCR and plotted against a reference gene, TATA-box Binding protein (TBP). Results show that AAV9.AAA.VQVGRTS (NVG07) had lower expression in the liver. FIGS. 13 A-B.

C57B16 mice (n=4 per administration group) were administered AAV9 capsids carrying a CAG.TdTomato transgene, 1E13 or 1E14 GC/kg IV administration per animal. Following 3 weeks, animals were sacrificed and brain, liver, bicep and gastrocnemius tissues were collected.

Separately, C57B16 mice (n=5 per administration group) were administered NVG07 capsids carrying a CAG.TdTomato transgene, 1E13 or 1E14 GC/kg IV administration per animal. Following 3 weeks, animals were sacrificed and brain, liver, tibialis anterior (TA) quadricep, bicep and gastrocnemius tissues were collected.

Brain slices from cortex, hippocampus and striatum tissues from both studies (AAV9 and NVG07) were also prepared for fluorescence imaging (tdTomato/DAPI). AAV9.AAA.NVG07 redirects tropism from astrocytes to neurons in the brain of the mouse (data not shown).

TdTomato genome (cDNA) and transcripts (mRNA) were detected by digital PCR and plotted against a reference gene, TATA-box Binding protein (TBP). Results are shown in Tables 6-13.

TABLE 6
AAV9/1E13/RNA-tdTom transcripts/TBP

| | | | | | |
|---|---|---|---|---|---|
| Brain | 0.036890311 | 0.111015937 | 0.086795623 | 0.199962374 | 0.284954529 |
| Liver | Not done (Nd) | Nd | Nd | Nd | Nd |
| Bicep | 0.434204944 | 0.692850013 | 0.576478591 | 1.572121792 | 1.280973036 |
| Gas | 0.681902249 | 0.671394813 | 0.254783067 | 2.665095667 | 0.33763115 |

TABLE 7
AAV9/1E13/DNA-tdTom/diploid cell

| | | | | | |
|---|---|---|---|---|---|
| Brain | 0.013450943 | 0.011279439 | 0.021593898 | 0.038522416 | 0.018567217 |
| Liver | Nd | Nd | Nd | Nd | Nd |
| Bicep | 0.058739945 | 0.095610192 | 0.101944179 | 0.549394202 | 0.107221812 |
| Gas | 0.077698166 | 0.048297601 | 0.03348689 | 0.073418602 | 0.085699073 |

TABLE 8
AAV9/1E14/RNA-tdTom transcripts/TBP

| | | | | | |
|---|---|---|---|---|---|
| Brain | 1.178050241 | 1.217263137 | 0.895701935 | 2.134230949 | 1.218719168 |
| Liver | Nd | Nd | Nd | Nd | Nd |
| Bicep | 15.35463383 | 2.358943003 | 10.23422082 | 60.35947847 | 29.1243009 |
| Gas | 40.62668523 | 18.44852392 | 5.587096873 | 60.87078632 | 10.39744051 |

TABLE 9
AAV9/1E14/DNA-tdTom/Diploid cell

| | | | | | |
|---|---|---|---|---|---|
| BRAIN | 0.278629609 | 0.431457095 | 0.228405767 | 0.333674887 | 0.220072005 |
| LIVER | 240.8114 | 328.9249 | 220.2679 | 125.229 | 226.3849 |
| BICEP | 1.08169672 | 0.743156382 | 0.676354039 | 0.912752148 | 1.058356597 |
| GAS | 0.394223444 | 2.054787079 | 0.932967195 | 0.606069664 | 1.117394992 |

TABLE 10
NVG07/1E13/RNA-tdTom Transcripts/TBP

| | | | | | |
|---|---|---|---|---|---|
| BRAIN | 0.00375462 | 0.028063924 | 0.020756832 | 0.032251626 | 0.01364307 |
| LIVER | 3.047034456 | 8.11774917 | 2.214509582 | 5.013711529 | 1.927788473 |
| TA | 0.078822571 | 0.126637776 | 0.367988038 | 0.696732748 | 0.053506641 |
| QUAD | 0.144743958 | 0.18249131 | 0.834414732 | 0.760439126 | 0.124937345 |
| BICEP | 0.028115576 | 0.131026526 | 0.174242523 | 0.152152957 | 0.056497243 |
| GAS | 0.101910779 | 0.727130082 | 1.050164729 | 0.382196398 | 0.173578122 |

TABLE 11
NVG07/1E13/DNA-tdTom/Diploid cell

| | | | | | |
|---|---|---|---|---|---|
| BRAIN | 0.000488948 | 0.002704717 | 0.002265579 | 0.003374586 | 0.000431437 |
| LIVER | 0.047667814 | 0.217529774 | 0.148715088 | 0.251105783 | 0.069879203 |
| T.A. | 0.003982067 | 0.010817625 | 0.012242956 | 0.027075116 | 0.006865514 |
| QUAD | 0.004530628 | 0.006238941 | 0.058679912 | 0.051015036 | 0.010696398 |
| BICEP | 0.00332028 | 0.014682863 | 0.019468111 | 0.050395935 | 0.005424901 |
| GAS | 0.003626522 | 0.011423566 | 0.014696037 | 0.028799631 | 0.005488395 |

TABLE 12
NVG07/1E14/RNA-tdTom Transcripts/TBP

| | | | | | |
|---|---|---|---|---|---|
| BRAIN | 1.509950773 | 0.415176657 | 0.287061103 | 0.574066385 | 0.02201578* |
| LIVER | 5.921066892 | 9.549919727 | 2.540978226 | 5.791713751 | 1.694483204* |
| TA | 5.29366453 | 11.09910504 | 14.08705972 | 16.80101394 | 0.058042404* |
| QUAD | 34.15055202 | 16.94971485 | 6.788304343 | 22.18649393 | 0.099859085* |
| BICEP | 3.004314619 | 10.03525839 | 6.499347379 | 20.83190632 | 0.040049364* |
| GAS | 13.10956254 | 26.67521715 | 23.65668044 | 59.21139309 | 0.066293391* |

TABLE 13
NVG07/1E14/DNA-tdTom/Diploid cell

| | | | | | |
|---|---|---|---|---|---|
| BRAIN | 0.022000771 | 0.022543271 | 0.011689717 | 0.049770374 | 0.000542091* |
| LIVER | 5.935441167 | 11.24125919 | 0.718609553 | 6.158077504 | 0.055109823* |
| T.A. | 0.19740364 | 0.162904378 | 0.278037585 | 0.285422593 | 0.010024539* |
| QUAD | 0.233550486 | 0.198675241 | 1.27587749 | 0.523329496 | 0.004910094* |
| BICEP | 0.128187391 | 0.196211218 | 0.234569107 | 0.611000095 | 0.005177627* |
| GAS | 0.103334772 | 0.159689351 | 0.158928168 | 0.277316254 | 0.006636086* |

At the high dose (1E14 GC/kg) of single-vector administered to mice in this study, NVG07 yielded 40-fold lower genome DNA than AAV9 in liver, yet had essentially equivalent, or slightly lower DNA and RNA, observed than AAV9 in skeletal muscle RNA. Lower liver expression of certain AAV gene therapy transgenes is desirable. Currently, several challenges for AAV therapeutics include potential adverse effects to liver function and immune responses for the patient. For a more systemic distribution such as for a muscle disease, approved AAV therapeutics are administered intravenously in high doses, typically greater than 1×10¹³ (1E13) genome copies per kilogram of body weight of the patient (vector genomes (vg)=genome copies (GC)), e.g. delandistrogene moxeparvovec administered at 1.33E14 vector genomes (vg)/kg of body weight. An AAV therapeutic having less distribution to the liver, where liver transduction is not warranted or may be the cause of adverse reactions, would prove beneficial to patients, especially if other properties of the AAV therapy convey the intended efficacy. Based on the finding that NVG07 exhibited higher observable neuronal expression (by fluorescent imaging) than AAV9, which transduced mostly astrocytes, the NVG07 capsid also provides an advantage for neuromuscular diseases where neuronal expression of an AAV-delivered transgene would be warranted.

### 6.6. Example 6—Evaluation and Selection of Capsids from a Library

Another round of peptides were selected from the B4 pool and administered to NHP as described above.

TABLE 14

| Capsid Name | Diaphragm (mRNA) | | GAS (mRNA) | | Quad (mRNA) | | TA (mRNA) | | Bicep (mRNA) | |
|---|---|---|---|---|---|---|---|---|---|---|
| (peptide SEQ ID NO) | NHP 4001 | NHP 4003 | NHP 4001 | NHP 4003 | NHP 4001 | NHP 4003 | NHP 4001 | NHP 4003 | NHP 4001 | NHP 4003 |
| AAV5 | 0.020035 | 0 | 0.0000142 | 0 | 0.0000796 | 0 | 0.001013 | 0 | 0.001892 | 0.002466 |
| AAV9.AAA | 0 | 0.014883 | 0.002098 | 0.015461 | 0.001959 | 0.00104 | 0.027122 | 0.032811 | 0.007654 | 0.010425 |
| AAV10 | 0 | 0.007773 | 0.000175 | 0.008254 | 0 | 0.002042 | 0 | 0.005185 | 0 | 0.000915 |
| AAV11 | 0 | 0 | 0.0000292 | 0 | 0.0000256 | 0 | 0.001255 | 0 | 0 | 0 |
| AAV12 | 0.004601 | 0 | 0.005313 | 0 | 0.003358 | 0 | 0 | 0 | 0.003805 | 0 |
| AAV13 | 0 | 0 | 0.00000578 | 0 | 0 | 0 | 0 | 0 | 0 | 0 |
| AAV8 | 0 | 0.009535 | 0.002891 | 0.010214 | 0.0000676 | 0.011017 | 0 | 0 | 0 | 0.009527 |
| AAV9 | 0.092047 | 0.047408 | 0.036513 | 0.041086 | 0.043732 | 0.06434 | 0.115277 | 0.077566 | 0.126715 | 0.081175 |
| AAV9.AAA.NVG07 | 0.073887 | 0.02668 | 0.036421 | 0.017508 | 0.033896 | 0.019427 | 0.028239 | 0.015125 | 0.063989 | 0.015024 |
| (SEQ ID NO: 8) | | | | | | | | | | |
| AAV9.455.DGRRIGV | 0 | 0.012639 | 0.062059 | 0.060865 | 0.056926 | 0.008295 | 0 | 0 | 0.030957 | 0.042943 |
| (SEQ ID NO: 116) | | | | | | | | | | |
| AAV9.455.TSGERRG | 0.018763 | 0.02622 | 0.026409 | 0.017124 | 0.035988 | 0.015875 | 0.012235 | 0.026689 | 0.015389 | 0.017502 |
| (SEQ ID NO: 117) | | | | | | | | | | |
| AAV9.455.PSSVQHR | 0.072458 | 0.063784 | 0.082454 | 0.031916 | 0.099667 | 0.041616 | 0.048056 | 0.0507 | 0.055866 | 0.02608 |
| (SEQ ID NO: 118) | | | | | | | | | | |
| AAV9.455.SSSVQHR | 0.010228 | 0.032451 | 0.037711 | 0.022562 | 0.041583 | 0.029147 | 0.025958 | 0.027321 | 0.026488 | 0.019774 |
| (SEQ ID NO: 119) | | | | | | | | | | |
| AAV9.455.SGMQERR | 0.006042 | 0.030419 | 0.027733 | 0.016933 | 0.029535 | 0.00861 | 0.005449 | 0.010547 | 0.004598 | 0.004968 |
| (SEQ ID NO: 120) | | | | | | | | | | |
| AAV9.455.SIAKNSA | 0 | 0.00745 | 0.006203 | 0.028216 | 0.004649 | 0.011929 | 0.008024 | 0.026454 | 0.004576 | 0.018454 |
| (SEQ ID NO: 76) | | | | | | | | | | |
| AAV9.455.LERGNLE | 0 | 0.003682 | 0.001149 | 0.005734 | 0.001289 | 0.011628 | 0.004441 | 0.014179 | 0.006002 | 0.00535 |
| (SEQ ID NO: 121) | | | | | | | | | | |
| AAV9.455.YRDVRQT | 0 | 0.047833 | 0.011339 | 0.048707 | 0.002079 | 0.021824 | 0.000879 | 0.038864 | 0.000976 | 0.028987 |
| (SEQ ID NO: 122) | | | | | | | | | | |
| AAV9.455.PSAVQHR | 0.026604 | 0.029804 | 0.0506 | 0.027253 | 0.05717 | 0.021541 | 0.007626 | 0.027586 | 0.037618 | 0.009481 |
| (SEQ ID NO: 123) | | | | | | | | | | |
| AAV9.455.AQVGRAS | 0.088632 | 0.046325 | 0.038686 | 0.031259 | 0.049969 | 0.038609 | 0.093481 | 0.024328 | 0.055467 | 0.044654 |
| (SEQ ID NO: 24) | | | | | | | | | | |
| AAV9.455.SVTSVRV | 0.017695 | 0.032461 | 0.027767 | 0.028252 | 0.022977 | 0.029828 | 0.015916 | 0.024275 | 0.009741 | 0.034885 |
| (SEQ ID NO: 37) | | | | | | | | | | |
| AAV9.455.YVGGRAV | 0.047559 | 0.08009 | 0.091561 | 0.056149 | 0.114782 | 0.062423 | 0.089687 | 0.057371 | 0.063298 | 0.061322 |
| (SEQ ID NO: 124) | | | | | | | | | | |
| AAV9.455.MKYRGTD | 0.020127 | 0.013741 | 0.028148 | 0.01385 | 0.035928 | 0.019683 | 0.029912 | 0.021126 | 0.034469 | 0.014141 |
| (SEQ ID NO: 45) | | | | | | | | | | |
| AAV9.455.HMGQYGK | 0 | 0.034439 | 0.014707 | 0.017065 | 0.003132 | 0.028222 | 0 | 0.029524 | 0 | 0.033974 |
| (SEQ ID NO: 82) | | | | | | | | | | |
| AAV9.455.IGSRGVA | 0.025609 | 0.041979 | 0.04564 | 0.055189 | 0.053971 | 0.04909 | 0.063916 | 0.035789 | 0.04762 | 0.054291 |
| (SEQ ID NO: 125) | | | | | | | | | | |
| AAV9.455.SDVSRPR | 0.020171 | 0.01173 | 0.088732 | 0.012306 | 0.099453 | 0.003151 | 0.026286 | 0.003998 | 0.033182 | 0.00384 |
| (SEQ ID NO: 126) | | | | | | | | | | |
| AAV9.455.GSVRQAA | 0.009634 | 0.079906 | 0.035125 | 0.073909 | 0.022702 | 0.067478 | 0.033772 | 0.094574 | 0.006611 | 0.082951 |
| (SEQ ID NO: 127) | | | | | | | | | | |
| AAV9.455.QSPHTSQ | 0.035755 | 0.025078 | 0.006733 | 0.019373 | 0.006917 | 0.035874 | 0.012861 | 0.02567 | 0.016607 | 0.017489 |
| (SEQ ID NO: 128) | | | | | | | | | | |
| AAV9.455.ASQAYHG | 0 | 0.024187 | 0.003581 | 0.058372 | 0.000812 | 0.054513 | 0 | 0.02941 | 0 | 0.043668 |
| (SEQ ID NO: 129) | | | | | | | | | | |

0 = Not Detected

In the B4 study of pooled vectors, several vectors having peptide inserts (e.g. DGRRIGV, YVGGRAV, PSSVQHR, IGSRGVA, GSVRQAA, SDVSRPR, AQVGRAS) exhibited higher mRNA expression in skeletal muscle tissues (such as gastrocnemius) than AAV9.

### 6.7. Example 7—Evaluation of a Consensus Sequence

Highly similar sequences and motifs were found amongst the peptides that transduced muscle efficiently while de-targeting the liver in the AAV9.AAA capsid library.

The “Fold-difference” value in the chart represents the fold-difference in muscle tropism for the capsid with the peptide compared to its parental. As indicated in Table 15, AAV9.AAA capsids comprising these peptides which have similar amino acid motifs convey greater than 2-fold up to 27-fold greater transduction in muscle compared to an AAV9.AAA capsid.

TABLE 15

| | Input | Number | | | Fold- |
|---|---|---|---|---|---|
| Sequence | Counts | Samples | Mean | CV | difference |
| VQVGRTS (SEQ ID NO: 8) | 180 | 7 | 11.52 | 1.43 | 26.84 |
| VQVGRSS (SEQ ID NO: 44) | 199 | 6 | 3.16 | 1.56 | 7.37 |
| VQVGRVS (SEQ ID NO: 36) | 254 | 5 | 4.32 | 1.50 | 10.07 |
| VQVGRYS (SEQ ID NO: 130) | 93 | 2 | 1.12 | 2.56 | 2.61 |
| VQVGRAS (SEQ ID NO: 131) | 172 | 1 | 1.03 | 3.32 | 2.39 |
| VQVGRPS (SEQ ID NO: 132) | 81 | 1 | 0.99 | 3.32 | 2.30 |
| AQVGRAS (SEQ ID NO: 24) | 171 | 8 | 7.47 | 1.65 | 17.4 |
| VQVGRAA (SEQ ID NO: 5) | 86 | 4 | 11.79 | 2.10 | 27.47 |
| VQVVRPS (SEQ ID NO: 133) | 63 | 3 | 6.90 | 2.37 | 16.09 |
| VQVGHAS (SEQ ID NO: 134) | 152 | 3 | 1.27 | 2.92 | 2.96 |
| NO INSERT | 2103 | 5 | 0.43 | 1.35 | 1.00 |

The consensus sequence SEQ ID NO: 135 is X₁-Q-V-X₂-X₃-X₄-X₅, wherein X₁ is V or A, X₂ is S, G, V or A, X₃ is R or H, X₄ is any amino acid and X₅ is S, G, V or A. The consensus sequence SEQ ID NO: 136 is X₁-Q-V-X₂-X₃-X₄-X₅, wherein X₁ is V or A, X₂ is S, G, V or A, X₃ is R or H, X₄ is any amino acid and X₅ is S or A. The consensus sequence SEQ ID NO: 137 is X₁-Q-V-X₂-X₃-X₄-X₅, wherein X₁ is V or A, X₂ is S, G, V or A, X₃ is R or H, X₄ is T, S, V, Y, A or P and X₅ is S, G, V or A. The consensus sequence SEQ ID NO: 138 is X₁-Q-V-X₂-X₃-X₄-X₅, wherein X₁ is V or A, X₂ is S, G, V or A. X₃ is R or H, X₄ is T, S, V, Y, A or P and X₅ is S or A.

TABLE 16
Consensus Peptide Sequences

| SEQ ID | Consensus Peptide | | | | | |
|---|---|---|---|---|---|---|
| NO: | Sequences | X₁ | X₂ | X₃ | X₄ | X₅ |
| 135 | X₁-Q-V-X₂-X₃-X₄-X₅ | V or A | S, G, V or A | R or H | Any Amino | S, G, V or A |
| | | | | | Acid | |
| 136 | X₁-Q-V-X₂-X₃-X₄-X₅ | V or A | S, G, V or A | R or H | Any Amino | S or A |
| | | | | | Acid | |
| 137 | X₁-Q-V-X₂-X₃-X₄-X₅ | V or A | S, G, V or A | R or H | T, S, V, Y, A | S, G, V or A |
| | | | | | or P | |
| 138 | X₁-Q-V-X₂-X₃-X₄-X₅ | V or A | S, G, V or A | R or H | T, S, V, Y, A | S or A |
| | | | | | or P | |

### 6.7.1.1 Conclusions

AAV capsid modifications performed by random peptide insertions in surface exposed loop of VR-IV were able to produce sufficient library titers in the production system described herein for analysis of transduction properties in NHP tissues. The method provided for reducing carryover parental plasmid following cloning of the random insertions and was shown to reduce overrepresentation of the parental vector in the library biodistribution and transduction studies.

Intravenous administration of AAV9.AAA.VR4 to NHPs resulted in a number of higher relative abundance engineered vectors in muscle suitable for use as a gene therapy vector carrying a gene of interest to treat a muscle disorder.

TABLE 17
Capsid Amino Acid Sequences

| Capsid | Insert/ | |
|---|---|---|
| Name | modification | Amino Acid Sequence |
| AAV1 | n/a | MAADGYLPDWLEDNLSEGIREWWDLKPGAPKPKANQQKQDDGRGLVLPGYKYLGPFNGLDKGEPV |
| | | NAADAAALEHDKAYDQQLKAGDNPYLRYNHADAEFQERLQEDTSFGGNLGRAVFQAKKRVLEPLG |
| | | LVEEGAKTAPGKKRPVEQSPQEPDSSSGIGKTGQQPAKKRLNFGQTGDSESVPDPQPLGEPPATP |
| | | AAVGPTTMASGGGAPMADNNEGADGVGNASGNWHCDSTWLGDRVITTSTRTWALPTYNNHLYKQI |
| | | SSASTGASNDNHYFGYSTPWGYFDFNRFHCHFSPRDWQRLINNNWGFRPKRLNFKLFNIQVKEVT |
| | | TNDGVTTIANNLTSTVQVFSDSEYQLPYVLGSAHQGCLPPFPADVFMIPQYGYLTLNNGSQAVGR |
| | | SSFYCLEYFPSQMLRTGNNFTFSYTFEEVPFHSSYAHSQSLDRLMNPLIDQYLYYLNRTQNQSGS |
| | | AQNKDLLFSRGSPAGMSVQPKNWLPGPCYRQQRVSKTKTDNNNSNFTWTGASKYNLNGRESIINP |
| | | GTAMASHKDDEDKFFPMSGVMIFGKESAGASNTALDNVMITDEEEIKATNPVATERFGTVAVNFQ |
| | | SSSTDPATGDVHAMGALPGMVWQDRDVYLQGPIWAKIPHTDGHFHPSPLMGGFGLKNPPPQILIK |
| | | NTPVPANPPAEFSATKFASFITQYSTGQVSVEIEWELQKENSKRWNPEVQYTSNYAKSANVDFTV |
| | | DNNGLYTEPRPIGTRYLTRPL (SEQ ID NO: 139) |
| AAV2 | n/a | MAADGYLPDWLEDTLSEGIRQWWKLKPGPPPPKPAERHKDDSRGLVLPGYKYLGPFNGLDKGEPV |
| | | NEADAAALEHDKAYDRQLDSGDNPYLKYNHADAEFQERLKEDTSFGGNLGRAVFQAKKRVLEPLG |
| | | LVEEPVKTAPGKKRPVEHSPVEPDSSSGTGKAGQQPARKRLNFGQTGDADSVPDPQPLGQPPAAP |
| | | SGLGTNTMATGSGAPMADNNEGADGVGNSSGNWHCDSTWMGDRVITTSTRTWALPTYNNHLYKQI |
| | | SSQSGASNDNHYFGYSTPWGYFDFNRFHCHFSPRDWQRLINNNWGFRPKRLNFKLFNIQVKEVTQ |
| | | NDGTTTIANNLTSTVQVFTDSEYQLPYVLGSAHQGCLPPFPADVFMVPQYGYLTLNNGSQAVGRS |
| | | SFYCLEYFPSQMLRTGNNFTFSYTFEDVPFHSSYAHSQSLDRLMNPLIDQYLYYLSRTNTPSGTT |
| | | TQSRLQFSQAGASDIRDQSRNWLPGPCYRQQRVSKTSADNNNSEYSWTGATKYHLNGRDSLVNPG |
| | | PAMASHKDDEEKFFPQSGVLIFGKQGSEKTNVDIEKVMITDEEEIRTTNPVATEQYGSVSTNLQR |
| | | GNRQAATADVNTQGVLPGMVWQDRDVYLQGPIWAKIPHTDGHFHPSPLMGGFGLKHPPPQISIKN |
| | | TPVPANPSTTFSAAKFASFITQYSTGQVSVEIEWELQKENSKRWNPEIQYTSNYNKSVNVDFTVD |
| | | INGVYSEPRPIGTRYLTR (SEQ ID NO: 140) |
| AAV3 | n/a | MAADGYLPDWLEDNLSEGIREWWALKPGVPQPKANQQHQDNRRGLVLPGYKYLGPGNGLDKGEPV |
| | | NEADAAALEHDKAYDQQLKAGDNPYLKYNHADAEFQERLQEDTSFGGNLGRAVFQAKKRILEPLG |
| | | LVEEAAKTAPGKKGAVDQSPQEPDSSSGVGKSGKQPARKRLNFGQTGDSESVPDPQPIGEPPAAP |
| | | TSLGSNTMASGGGAPMADNNEGADGVGNSSGNWHCDSQWLGDRVITTSTRTWALPTYNNHLYKQI |
| | | SSQSGASNDNHYFGYSTPWGYFDFNRFHCHFSPRDWQRLINNNWGFRPKKLSFKLFNIQVRGVTQ |
| | | NDGTTTIANNLTSTVQVFTDSEYQLPYVLGSAHQGCLPPFPADVFMVPQYGYLTLNNGSQAVGRS |
| | | SFYCLEYFPSQMLRTGNNFQFSYTFEDVPFHSSYAHSQSLDRLMNPLIDQYLYYLNRTQGTTSGT |
| | | TNQSRLLFSQAGPQSMSLQARNWLPGPCYRQQRLSKTANDNNNSNFPWTAASKYHLNGRDSLVNP |
| | | GPAMASHKDDEEKFFPMHGNLIFGKEGTTASNAELDNVMITDEEEIRTTNPVATEQYGTVANNLQ |
| | | SSNTAPTTGTVNHQGALPGMVWQDRDVYLQGPIWAKIPHTDGHFHPSPLMGGFGLKHPPPQIMIK |
| | | NTPVPANPPTTFSPAKFASFITQYSTGQVSVEIEWELQKENSKRWNPEIQYTSNYNKSVNVDFTV |
| | | DTNGVYSEPRPIGTRYLTRNL (SEQ ID NO: 141) |
| AAV4 | n/a | MTDGYLPDWLEDNLSEGVREWWALQPGAPKPKANQQHQDNARGLVLPGYKYLGPGNGLDKGEPVN |
| | | AADAAALEHDKAYDQQLKAGDNPYLKYNHADAEFQQRLQGDTSFGGNLGRAVFQAKKRVLEPLGL |
| | | VEQAGETAPGKKRPLIESPQQPDSSTGIGKKGKQPAKKKLVFEDETGAGDGPPEGSTSGAMSDDS |
| | | EMPAAAGGAAVEGGQGADGVGNASGDWHCDSTWSEGHVTTTSTRTWVLPTYNNHLYKRLGESLQS |
| | | NTYNGFSTPWGYFDFNRFHCHFSPRDWQRLINNNWGMRPKAMRVKIFNIQVKEVTTSNGETTVAN |
| | | NLTSTVQIFADSSYELPYVMDAGQEGSLPPFPNDVFMVPQYGYCGLVTGNTSQQQTDRNAFYCLE |
| | | YFPSQMLRTGNNFEITYSFEKVPFHSMYAHSQSLDRLMNPLIDQYLWGLQSTTTGTTINAGTATT |
| | | NFTKLRPTNFSNFKKNWLPGPSIKQQGFSKTANQNYKIPATGSDSLIKYETHSTLDGRWSALTPG |
| | | PPMATAGPADSKFSNSQLIFAGPKQNGNTATVPGTLIFTSEEELAATNATDTDMWGNLPGGDQSN |
| | | SNLPTVDRLTALGAVPGMVWQNRDIYYQGPIWAKIPHTDGHFHPSPLIGGFGLKHPPPQIFIKNT |
| | | PVPANPATTFSSTPVNSFITQYSTGQVSVQIDWEIQKERSKRWNPEVQFTSNYGQQNSLLWAPDA |
| | | AGKYTEPRAIGTRYLTHHL (SEQ ID NO: 142) |
| AAV5 | n/a | MSFVDHPPDWLEEVGEGLREFLGLEAGPPKPKPNQQHQDQARGLVLPGYNYLGPGNGLDRGEPVN |
| | | RADEVAREHDISYNEQLEAGDNPYLKYNHADAEFQEKLADDTSFCCNLCKAVFQAKKRVLEPFGL |
| | | VEEGAKTAPTGKRIDDHFPKRKKARTEEDSKPSTSSDAEAGPSGSQQLQIPAQPASSIGADTMSA |
| | | GGGGPLGDNNQGADGVGNASGDWHCDSTWMGDRVVTKSTRTWVLPSYNNHQYREIKSGSVDGSNA |
| | | NAYFGYSTPWGYFDFNRFHSHWSPRDWQRLINNYWGFRPRSLRVKIFNIQVKEVTVQDSTTTIAN |
| | | NLTSTVQVFTDDDYQLPYVVGNGTEGCLPAFPPQVFTLPQYGYATLNRDNTENPTERSSFFCLEY |
| | | FPSKMLRTGNNFEFTYNFEEVPFHSSFAPSQNLFKLANPLVDQYLYRFVSTNNTGGVQFNKNLAG |
| | | RYANTYKNWFPGPMGRTQGWNLGSGVNRASVSAFATTNRMELEGASYQVPPQPNGMINNLQGSNT |
| | | YALENTMIFNSQPANPGTTATYLEGNMLITSESETQPVNRVAYNVGGQMATNNQSSTTAPATGTY |
| | | NLQEIVPGSVWMERDVYLQGPIWAKIPETGAHFHPSPAMGGFGLKHPPPMMLIKNTPVPGNITSF |
| | | SDVPVSSFITQYSTGQVTVEMEWELKKENSKRWNPEIQYTNNYNDPQFVDFAPDSTGEYRTTRPI |
| | | GTRYLTRPL (SEQ ID NO: 143) |
| AAV6 | n/a | MAADGYLPDWLEDNLSEGIREWWDLKPGAPKPKANQQKQDDGRGLVLPGYKYLGPFNGLDKGEPV |
| | | NAADAAALEHDKAYDQQLKAGDNPYLRYNHADAEFQERLQEDTSFGGNLGRAVFQAKKRVLEPFG |
| | | LVEEGAKTAPGKKRPVEQSPQEPDSSSGIGKTGQQPAKKRLNFGQTGDSESVPDPQPIGEPPATP |
| | | AAVGPTTMASGGGAPMADNNEGADGVGNASGNWHCDSTWLGDRVITTSTRTWALPTYNNHLYKQI |
| | | SSASTGASNDNHYFGYSTPWGYFDFNRFHCHFSPRDWQRLINNNWGFRPKRLNFKLFNIQVKEVT |
| | | TNDGVTTIANNLTSTVQVFSDSEYQLPYVLGSAHQGCLPPFPADVFMIPQYGYLTLNNGSQAVGR |
| | | SSFYCLEYFPSQMLRTGNNFTFSYTFEDVPFHSSYAHSQSLDRLMNPLIDQYLYYLNRTQNQSGS |
| | | AQNKDLLFSRGSPAGMSVQPKNWLPGPCYRQQRVSKTKTDNNNSNFTWTGASKYNLNGRESIINP |
| | | GTAMASHKDDKDKFFPMSGVMIFGKESAGASNTALDNVMITDEEEIKATNPVATERFGTVAVNLQ |
| | | SSSTDPATGDVHVMGALPGMVWQDRDVYLQGPIWAKIPHTDGHFHPSPLMGGFGLKHPPPQILIK |
| | | NTPVPANPPAEFSATKFASFITQYSTGQVSVEIEWELQKENSKRWNPEVQYTSNYAKSANVDFTV |
| | | DNNGLYTEPRPIGTRYLTRPL (SEQ ID NO: 144) |
| AAV7 | n/a | MAADGYLPDWLEDNLSEGIREWWDLKPGAPKPKANQQKQDNGRGLVLPGYKYLGPFNGLDKGEPV |
| | | NAADAAALEHDKAYDQQLKAGDNPYLRYNHADAEFQERLQEDTSFGGNLGRAVFQAKKRVLEPLG |
| | | LVEEGAKTAPAKKRPVEPSPQRSPDSSTGIGKKGQQPARKRLNFGQTGDSESVPDPQPLGEPPAA |
| | | PSSVGSGTVAAGGGAPMADNNEGADGVGNASGNWHCDSTWLGDRVITTSTRTWALPTYNNHLYKQ |
| | | ISSETAGSTNDNTYFGYSTPWGYFDFNRFHCHFSPRDWQRLINNNWGFRPKKLRFKLFNIQVKEV |
| | | TTNDGVTTIANNLTSTIQVFSDSEYQLPYVLGSAHQGCLPPFPADVFMIPQYGYLTLNNGSQSVG |
| | | RSSFYCLEYFPSQMLRTGNNFEFSYSFEDVPFHSSYAHSQSLDRLMNPLIDQYLYYLARTQSNPG |
| | | GTAGNRELQFYQGGPSTMAEQAKNWLPGPCFRQQRVSKTLDQNNNSNFAWTGATKYHLNGRNSLV |
| | | NPGVAMATHKDDEDRFFPSSGVLIFGKTGATNKTTLENVLMTNEEEIRPTNPVATEEYGIVSSNL |
| | | QAANTAAQTQVVNNQGALPGMVWQNRDVYLQGPIWAKIPHTDGNFHPSPLMGGFGLKHPPPQILI |
| | | KNTPVPANPPEVFTPAKFASFITQYSTGQVSVEIEWELQKENSKRWNPEIQYTSNFEKQTGVDFA |
| | | VDSQGVYSEPRPIGTRYLTRNL (SEçID NO: 145) |
| AAV8 | n/a | MAADGYLPDWLEDNLSEGIREWWALKPGAPKPKANQQKQDDGRGLVLPGYKYLGPFNGLDKGEPV |
| | | NAADAAALEHDKAYDQQLQAGDNPYLRYNHADAEFQERLQEDTSFGGNLGRAVFQAKKRVLEPLG |
| | | LVEEGAKTAPGKKRPVEPSPQRSPDSSTGIGKKGQQPARKRLNFGQTGDSESVPDPQPLGEPPAA |
| | | PSGVGPNTMAAGGGAPMADNNEGADGVGSSSGNWHCDSTWLGDRVITTSTRTWALPTYNNHSYKQ |
| | | ISNGTSGGATNDNTYFGYSTPWGYFDFNRFHCHFSPRDWQRLINNNWGFRPKRLSFKIFNIQVKE |
| | | VTQNEGTKTIANNLTSTIQVFTDSEYQLPYVLGSAHQGCLPPFPADVFMIPQYGYLTINNGSQAV |
| | | GRSSFYCLEYFPSQMLRTGNNFQFTYTFEDVPFHSSYAHSQSLDRLMNPLIDQYLYYLSRTQTTG |
| | | GTANTQTLGFSQGGPNTMANQAKNWLPGPCYRQQRVSTTTGQNNNSNFAWTAGTKYHLNGRNSLA |
| | | NPGIAMATHKDDEERFFPSNGILIFGKQNAARDNADYSDVMLTSEEEIKTTNPVATEEYGIVADN |
| | | LQQQNTAPQIGTVNSQGALPGMVWQNRDVYLQGPIWAKIPHTDGNFHPSPLMGGFGLKHPPPQIL |
| | | IKNTPVPADPPTTFNQSKLNSFITQYSTGQVSVEIEWELQKENSKRWNPEIQYTSNYYKSTSVDF |
| | | AVNTEGVYSEPRPIGTRYLTRNL (SEQ ID NO: 146) |
| AAVhu31 | n/a | MAADGYLPDWLEDTLSEGIRQWWKLKPGPPPPKPAERHKDDSRGLVLPGYKYLGPGNGLDKGEPV |
| | | NAADAAALEHDKAYDQQLKAGDNPYLKYNHADAEFQERLKEDTSFGGNLGRAVFQAKKRLLEPLG |
| | | LVEEAAKTAPGKKRPVEQSPQEPDSSAGIGKSGSQPAKKKLNFGQTGDTESVPDPQPIGEPPAAP |
| | | SGVGSLTMASGGGAPVADNNEGADGVGSSSGNWHCDSQWLGDRVITTSTRTWALPTYNNHLYKQI |
| | | SNSTSGGSSNDNAYFGYSTPWGYFDFNRFHCHFSPRDWQRLINNNWGFRPKRLNFKLFNIQVKEV |
| | | TDNNGVKTIANNLTSTVQVFTDSDYQLPYVLCSAHECCLPPFPADVFMIPQYGYLTLNDGGQAVG |
| | | RSSFYCLEYFPSQMLRTGNNFQFSYEFENVPFHSSYAHSQSLDRLMNPLIDQYLYYLSKTINGSG |
| | | QNQQTLKFSVAGPSNMAVQGRNYIPGPSYRQQRVSTTVTQNNNSEFAWPGASSWALNGRNSLMNP |
| | | GPAMASHKEGEDRFFPLSGSLIFGKQGTGRDNVDADKVMITNEEEIKTTNPVATESYGQVATNHQ |
| | | SAQAQAQTGWVQNQGILPGMVWQDRDVYLQGPIWAKIPHTDGNFHPSPLMGGFGMKHPPPQILIK |
| | | NTPVPADPPTAFNKDKLNSFITQYSTGQVSVEIEWELQKENSKRWNPEIQYTSNYYKSNNVEFAV |
| | | STEGVYSEPRPIGTRYLTRNL (SEQ ID NO: 147) |
| AAVhu32 | n/a | MAADGYLPDWLEDTLSEGIRQWWKLKPGPPPPKPAERHKDDSRGLVLPGYKYLGPGNGLDKGEPV |
| | | NAADAAALEHDKAYDQQLKAGDNPYLKYNHADAEFQERLKEDTSFGGNLGRAVFQAKKRLLEPLG |
| | | LVEEAAKTAPGKKRPVEQSPQEPDSSAGIGKSGSQPAKKKLNFGQTGDTESVPDPGQPIGEPPAA |
| | | PSGVGSLTMASGGGAPVADNNEGADGVGSSSGNWHCDSQWLGDRVITTSTRTWALPTYNNHLYKQ |
| | | ISNSTSGGSSNDNAYFGYSTPWGYFDFNRFHCHFSPRDWQRLINNNWGFRPKRLNFKLFNIQVKE |
| | | VTDNNGVKTIANNLTSTVQVFTDSDYCLPYVLGSAHEGCLPPFPADVFMIPQYGYLTLNDGSQAV |
| | | GRSSFYCLEYFPSQMLRTGNNFQFSYEFENVPEHSSYAHSQSLDRLMNPLIDQYLYYLSKTINGS |
| | | GQNQQTLKFSVAGPSNMAVQGRNYIPGPSYRQQRVSTTVTQNNNSEFAWPGASSWALNGRNSLMN |
| | | PGPAMASHKEGEDRFFPLSGSLIFGKCGTGRDNVDADKVMITNEEEIKTTNPVATESYGQVATNH |
| | | QSAQAQAQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKSNNVEFA |
| | | VNTEGVYSEPRPIGTRYLTRNL (SEQ ID NO: 148) |
| AAVrh39 | | MAADGYLPDWLEDNLSEGIREWWALKPGAPKPKANQQKQDDGRGLVLPGYKYLGPFNGLDKGEPV |
| | | NAADAAALEHDKAYDQQLKAGDNPYLRYNHADAEFQERLQEDTSFGGNLGRAVFQAKKRVLEPLG |
| | | LVEEAAKTAPGKKRPVEPSPQRSPDSSTGIGKKGQQPAKKRLNFGQTGDSESVPDPQPIGEPPAG |
| | | PSGLGSGTMAAGGGAPMADNNEGADGVGSSSGNWHCDSTWLGDRVITTSTRTWALPTYNNHLYKQ |
| | | ISNGTSGGSTNDNTYFGYSTPWGYFDFNRFHCHFSPRDWQRLINNNWGFRPKRLSFKLFNIQVKE |
| | | VTQNEGTKTIANNLTSTIQVFTDSEYQLPYVLGSAHQGCLPPFPADVFMIPQYGYLTLNNGSQAV |
| | | GRSSFYCLEYFPSQMLRTGNNFEFSYTFEDVPFHSSYAHSQSLDRLMNPLIDQYLYYLSRTQSTG |
| | | GTQGTQQLLFSQAGPANMSAQAKNWLPGPCYRQQRVSTTLSQNNNSNFAWTGATKYHLNGRDSLV |
| | | NPGVAMATHKDDEERFFPSSGVLMFGKQGAGRDNVDYSSVMLTSEEEIKTTNPVATEQYGVVADN |
| | | LQQTNTGPIVGNVNSQGALPGMVWQNRDVYLQGPIWAKIPHTDGNFHPSPLMGGFGLKHPPPQIL |
| | | IKNTPVPADPPTTFSQAKLASFITQYSTGQVSVEIEWELQKENSKRWNPEIQYTSNYYKSTNVDF |
| | | AVNTEGTYSEPRPIGTRYLTRNL (SEQ ID NO: 150) |
| AAV9 | n/a | MAADGYLPDWLEDNLSEGIREWWALKPGAPQPKANQQHQDNARGLVLPGYKYLGPGNGLDKGEPV |
| | | NAADAAALEHDKAYDQQLKAGDNPYLKYNHADAEFQERLKEDTSFGGNLGRAVFQAKKRLLEPLG |
| | | LVEEAAKTAPGKKRPVEQSPQEPDSSAGIGKSGAQPAKKRLNFGQTGDTESVPDPQPIGEPPAAP |
| | | SGVGSLTMASGGGAPVADNNEGADGVGSSSGNWHCDSQWLGDRVITTSTRTWALPTYNNHLYKQI |
| | | SNSTSGGSSNDNAYFGYSTPWGYFDFNRFHCHFSPRDWQRLINNNWGFRPKRLNFKLFNIQVKEV |
| | | TDNNGVKTIANNLISTVQVFTDSDYQLPYVLGSAHEGCLPPFPADVFMIPQYGYLTLNDGSQAVG |
| | | RSSFYCLEYFPSQMLRTGNNFQFSYEFENVPFHSSYAHSQSLDRLMNPLIDQYLYYLSKTINGSG |
| | | QNQQTLKFSVAGPSNMAVQGRNYIPGPSYRQQRVSTTVTQNNNSEFAWPGASSWALNGRNSLMNP |
| | | GPAMASHKEGEDRFFPLSGSLIFGKQGTGRDNVDADKVMITNEEEIKTTNPVATESYGQVATNHQ |
| | | SAQAQAQTGWVQNQGILPGMVWQDRDVYLQGPIWAKIPHTDGNFHPSPLMGGFGMKHPPPQILIK |
| | | NTPVPADPPTAFNKDKLNSFITQYSTGQVSVEIEWELQKENSKRWNPEIQYTSNYYKSNNVEFAV |
| | | NTEGVYSEPRPIGTRYLTRNL (SEQ ID NO: 151) |
| AAVrh10 | n/a | MAADGYLPDWLEDNLSEGIREWWDLKPGAPKPKANQQKQDDGRGLVLPGYKYLGPFNGLDKGEPV |
| | | NAADAAALEHDKAYDQQLKAGDNPYLRYNHADAEFQERLQEDTSFCCNLGRAVFQAKKRVLEPLG |
| | | LVEEGAKTAPGKKRPVEPSPQRSPDSSTGIGKKGQQPAKKRLNFGQTGDSESVPDPQPIGEPPAG |
| | | PSGLGSGTMAAGGGAPMADNNEGADGVGSSSGNWHCDSTWLGDRVITTSTRTWALPTYNNHSYKQ |
| | | ISNGTSGGSTNDNTYFGYSTPWGYFDFNRFHCHFSPRDWQRLINNNWGFRPKRLNFKLFNIQVKE |
| | | VTQNEGTKTIANNLTSTIQVFTDSEYQLPYVLGSAHQGCLPPFPADVFMIPQYGYLTLNNGSQAV |
| | | GRSSFYCLEYFPSQMLRTGNNFEFSYQFEDVPFHSSYAHSQSLDRLMNPLIDQYLYYLSRTQSTG |
| | | GTAGTQQLLFSQAGPNNMSAQAKNWLPGPCYRQQRVSTTLSQNNNSNFAWTGATKYHLNGRDSLV |
| | | NPGVAMATHKDDEERFFPSSGVLMFGKQGAGKDNVDYSSVMLTSEEEIKTTNPVATEQYGVVADN |
| | | LQQQNAAPIVGAVNSQGALPGMVWQNRDVYLQGPIWAKIPHTDGNFHPSPLMGGFGLKHPPPQIL |
| | | IKNTPVPADPPTTFSQAKLASFITQYSTGQVSVEIEWELQKENSKRWNPEIQYTSNYYKSTNVDF |
| | | AVNTDGTYSEPRPIGTRYLTRNL (SEQ ID NO: 152) |
| AAVhu37 | n/a | MAADGYLPDWLEDNLSEGIREWWDLKPGAPKPKANQQKQDDGRGLVLPGYKYLGPFNGLDKGEPV |
| | | NAADAAALEHDKAYDQQLKAGDNPYLRYNHADAEFQERLQEDTSFGGNLGRAVFQAKKRVLEPLG |
| | | LVEEAAKTAPGKKRPVEPSPQRSPDSSTGIGKKGQQPAKKRLNFGQTGDSESVPDPQPIGEPPAG |
| | | PSGLGSGTMAAGGGAPMADNNEGADGVGSSSGNWHCDSTWLGDRVITTSTRTWALPTYNNHSYKQ |
| | | ISNGTSGGSTNDNTYFGYSTPWGYFDFNRFHCHFSPRDWQRLINNNWGFRPKRLSFKLFNIQVKE |
| | | VTQNEGTKTIANNLTSTIQVFTDSEYQLPYVLGSAHQGCLPPFPADVFMIPQYGYLTLNNGSQAV |
| | | GRSSFYCLEYFPSQMLRTGNNFEFSYTFEDVPFHSSYAHSQSLDRLMNPLIDQYLYYLSRTQSTG |
| | | GTQGTQQLLFSQAGPANMSAQAKNWLPGPCYRQQRVSTTLSQNNNSNFAWTGATKYHLNGRDSLV |
| | | NPGVAMATHKDDEERFFPSSGVLMFGKQGAGRDNVDYSSVMLTSEEEIKTTNPVATEQYGVVADN |
| | | LQQTNTCPIVCNVNSQGALPCMVWQNRDVYJQCPIWAKIPHTDGNFHPSPLMGGFGLKHPPPQIL |
| | | IKNTPVPADPPTTFSQAKLASFITQYSTGQVSVEIEWELQKENSKRWNPEIQYTSNYYKSTNVDF |
| | | AVNTEGTYSEPRPIGTRYLTRNL (SEQ ID NO: 153) |
| AAV3B | n/a | MAADGYLPDWLEDNLSEGIREWWALKPGVPQPKANQQHQDNRRGLVLPGYKYLGPGNGLDKGEPV |
| | | NEADAAALEHDKAYDQQLKAGDNPYLKYNHADAEFQERLQEDTSFGGNLGRAVFQAKKRILEPLG |
| | | LVEEAAKTAPGKKRPVDQSPQEPDSSSGVGKSGKQPARKRLNFGQTGDSESVPDPQPLGEPPAAP |
| | | TSLGSNTMASGGGAPMADNNEGADGVGNSSGNWHCDSQWLGDRVITTSTRTWALPTYNNHLYKQI |
| | | SSQSGASNDNHYFGYSTPWGYFDFNRFHCHFSPRDWQRLINNNWGFRPKKLSFKLFNIQVKEVTQ |
| | | NDGTTTIANNLTSTVQVFTDSEYQLPYVLGSAHQGCLPPFPADVFMVPQYGYLTLNNGSQAVGRS |
| | | SFYCLEYFPSQMLRTGNNFQFSYTFEDVPFHSSYAHSQSLDRLMNPLIDQYLYYLNRTQGTTSGT |
| | | TNQSRLLFSQAGPQSMSLQARNWLPCPCYRQQRLSKTANDNNNSNFPWTAASKYHLNGRDSLVNP |
| | | GPAMASHKDDEEKFFPMHGNLIFGKEGTTASNAELDNVMITDEEEIRTTNPVATEQYGTVANNLQ |
| | | SSNTAPTTRTVNDQGALPGMVWQDRDVYLQGPIWAKIPHTDGHFHPSPLMGGFGLKHPPPQIMIK |
| | | NTPVPANPPTTFSPAKFASFITQYSTGQVSVELEWELQKENSKRWNPELQYTSNYNKSVNVDFTV |
| | | DTNGVYSEPRPIGTRYLTRNL (SEQ ID NO: 154) |
| AAVrh.20 | n/a | MAADGYLPDWLEDNLSEGIREWWDLKPGAPKPKANQQKQDDGRGLVLPGYKYLGPFNGLDKGEPV |
| | | NAADAAALEHDKAYDQQLKAGDNPYLRYNHADAEFQERLQEDTSFGGNLGRAVFQAKKRVLEPLG |
| | | LVEEGAKTAPGKKRPVEPSPQRSPDSSTGIGKTGQQPAKKRLNFGQTGDSESVPDPQPIGEPPAG |
| | | PSGLGSGTMAAGGGAPMADNNEGADGVGSSSGNWHCDSTWLGDRVITTSTRTWALPTYNNHLYKQ |
| | | ISNGTSGGSTNDNTYFGYSTPWGYFDFNRFHCHFSPRDWQRLINNNWGFRPKRLNFKLFNIQVKE |
| | | VTQNEGTKTIANNLTSTIQVFTDSEYQLPYVLGSAHQGCLPPFPADVFMIPQYGYLTLNNGSQAV |
| | | GRSSFYCLEYFPSQMLRTGNNFEFSYCFEDVPFHSSYAHSQSLDRLMNPLIDQYLYYLSRTQSTG |
| | | GTAGTQQLLFSQAGPNNMSAQAKNWLPGPCYRQQRVSTTLSQNNNSNFAWTGATKYHLNGRDSLV |
| | | NPGVAMATHKDDEERFFPSSGVLMFGKQGAGKDNVDYSSVMLTSEEEIKTTNPVATECYGVVADN |
| | | LQQQNAAPIVGAVNSQGALPGMVWQNRDVYLQGPIWAKIPHTDGNFHPSPLMGGFGLKHPPPQIL |
| | | IKNTPVPADPPTTFSQAKLASFITQYSTGQVSVEIEWELQKENSKRWNPEIQYTSNYYKSTNVDF |
| | | AVNTEGTYSEPRPIGTRYLTRNL (SEQ ID NO: 155) |
| AAVrh.74 | n/a | MAADGYLPDWLEDNLSEGIREWWDLKPGAPKPKANQQKQDNGRGLVLPGYKYLGPFNGLDKGEPV |
| version | | NAADAAALEHDKAYDQQLQAGDNPYLRYNHADAEFQERLQEDTSFGGNLGRAVFQAKKRVLEPLG |
| 1 | | LVESPVKTAPGKKRPVEPSPQRSPDSSTGIGKKGQQPAKKRLNFGQTGDSESVPDPQPIGEPPAG |
| | | PSGLGSGTMAAGGGAPMADNNEGADGVGSSSGNWHCDSTWLGDRVITTSTRTWALPTYNNHSYKQ |
| | | ISNGTSGGSTNDNTYFGYSTPWGYFDFNRFHCHFSPRDWQRLINNNWGFRPKRLNFKLFNIQVKE |
| | | VTQNEGTKTIANNLTSTIQVFTDSEYQLPYVLGSAHQGCLPPFPADVFMIPQYGYLTLNNGSQAV |
| | | GRSSFYCLEYFPSQMLRTGNNFEFSYNFEDVPFHSSYAHSQSLDRLMNPLIDQYLYYLSRTQSTG |
| | | GTAGTQQLLFSQAGPNNMSAQAKNWLPGPCYRQQRVSTTLSQNNNSNFAWTGATKYHLNGRDSLV |
| | | NPGVAMATHKDDEERFFPSSGVLMFGKQGAGKDNVDYSSVMLTSEEEIKTTNPVATECYGVVADN |
| | | LQQQNAAPIVGAVNSQGALPGMVWQNRDVYLQGPIWAKIPHTDGNFHPSPLMGGFGLKHPPPQIL |
| | | IKNTPVPADPPTTFTKAKLASFITQYSTGQVSVEIEWELQKENSKRWNPEIQYTSNYYKSTNVDF |
| | | AVNTEGTYSEPRPIGTRYLTRNL (SEQ ID NO: 156) |
| AAVrh.74 | n/a | MAADGYLPDWLEDNLSEGIREWWDLKPGAPKPKANQQKQDNGRGLVLPGYKYLGPFNGLDKGEPV |
| version | | NAADAAALEHDKAYDQQLQAGDNPYLRYNHADAEFQERLQEDTSFGGNLGRAVFQAKKRVLEPLG |
| 2 | | LVESPVKTAPGKKRPVEPSPQRSPDSSTGIGKKGQQPAKKRLNFGQTGDSESVPDPQPIGEPPAG |
| | | PSGLGSGTMAAGGGAPMADNNEGADGVGSSSGNWHCDSTWLGDRVITTSTRTWALPTYNNHLYKQ |
| | | ISNGTSGGSTNDNTYFGYSTPWGYFDFNRFHCHFSPRDWQRLINNNWGFRPKRLNFKLFNIQVKE |
| | | VTQNECTKTIANNLTSTIQVFTDSEYQLPYVLCSAHQGCLPPFPADVFMIPQYGYLTINNGSQAV |
| | | GRSSFYCLEYFPSQMLRTGNNFEFSYNFEDVPFHSSYAHSQSLDRLMNPLIDQYLYYLSRTQSTG |
| | | GTAGTQQLLFSQAGPNNMSAQAKNWLPGPCYRQQRVSTTLSQNNNSNFAWTGATKYHLNGRDSLV |
| | | NPGVAMATHKDDEERFFPSSGVLMFGKQGAGKDNVDYSSVMLTSEEEIKTTNPVATECYGVVADN |
| | | LQQQNAAPIVGAVNSQGALPGMVWQNRDVYLQGPIWAKIPHTDGNFHPSPLMGGFGLKHPPPQIL |
| | | IKNTPVPADPPTTFNQAKLASFITQYSTGQVSVEIEWELQKENSKRWNPEIQYTSNYYKSTNVDF |
| | | AVNTEGTYSEPRPIGTRYLTRNL (SEQ ID NO: 157) |
| AAV9.AAA | 496NNN498 | MAADGYLPDW LEDNLSEGIR EWWALKPGAP QPKANQQHQD NARGLVLPGY KYLGPGNGLD |
| | substitution | KGEPVNAADA AALEHDKAYD QQLKAGDNPY LKYNHADAEF QERLKEDTSF GGNLGRAVFQ |
| | to AAA | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 158) |
| AAV9.AAA. | SGTVIRS | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 1) | AKKRLLEPLG LVEEAAKTAP GKKRPVEQSP QEPDSSAGIG KSGAQPAKKR LNFGQTGDTE |
| 1 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSGTVIRS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEL EWELQKENSK RWNPELQYTS NYYKSNNVEF |
| | | AVNTEGVYSE PRPIGTRYLT RNL (SEQ ID NO: 159) |
| AAV9.AAA. | GRPAP | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 2) | AKKRLLEPLG LVEEAAKTAP GKKRPVEQSP QEPDSSAGIG KSGAQPAKKR LNFGQTGDTE |
| 2 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGRPAP GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 160) |
| AAV9.AAA. | KDQKIGP | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 3) | AKKRLLEPLG LVEEAAKTAP GKKRPVEQSP QEPDSSAGIG KSGAQPAKKR LNFGQTGDTE |
| 3 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSKDQKIGP GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 161) |
| AAV9.AAA. | TRQYVPG | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 4) | AKKRLLEPLG LVEEAAKTAP GKKRPVEQSP QEPDSSAGIG KSGAQPAKKR LNFGQTGDTE |
| 4 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSTRQYVPG GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 162) |
| AAV9.AAA. | VQVGRA | MAADGYLPDW LEDNLSEGIR EWWALKPGAP QPKANQQHQD NARGLVLPGY KYLGPGNGLD |
| VR4- | A | KGEPVNAADA AALEHDKAYD QQLKAGDNPY LKYNHADAEF QERLKEDTSF GGNLGRAVFQ |
| peptide | (SEQ ID | AKKRLLEPLG LVEEAAKTAP GKKRPVEQSP QEPDSSAGIG KSGAQPAKKR LNFGQTGDTE |
| 5 | NO: 5) | SVPDPQPIGE PPAAPSGVGS LTMASGGGAP VADNNEGADG VGSSSGNWHC DSQWLGDRVI |
| | and | TTSTRTWALP TYNNHLYKQI SNSTSGGSSN DNAYFGYSTP WGYFDFNRFH CHFSPRDWQR |
| | 496NNN498 | LINNNWGFRP KRLNFKLFNI QVKEVTDNNG VKTIANNLTS TVQVFTDSDY QLPYVLGSAH |
| | substitution | EGCLPPFPAD VFMIPQYGYL TLNDGSQAVG RSSFYCLEYF PSQMLRTGNN FQFSYEFENV |
| | to AAA | PFHSSYAHSQ SLDRLMNPLI DQYLYYLSKT INGSVQVGRAA GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 163) |
| AAV9.AAA. | FTQSRGG | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 6) | AKKRLLEPLG LVEEAAKTAP GKKRPVEQSP QEPDSSAGIG KSGAQPAKKR LNFGQTGDTE |
| 6 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSFTQSRGG GQNQQTLKFS VAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 164) |
| AAV9.AAA. | SRSDRPA | MAADGYLPDW LEDNLSEGIR EWWALKPGAP QPKANQQHQD NARGLVLPGY KYLGPGNGLD |
| VR4- | VR4- (SEQ ID | KGEPVNAADA AALEHDKAYD QQLKAGDNPY LKYNHADAEF QERLKEDTSF GGNLGRAVFQ |
| peptide | NO: 7) | AKKRLLEPLG LVEEAAKTAP GKKRPVEQSP QEPDSSAGIG KSGAQPAKKR LNFGQTGDTE |
| 7 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSRSDRPA GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 165) |
| AAV9.AAA. | VQVGRTS | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 8) | AKKRLLEPLG LVEEAAKTAP GKKRPVEQSP QEPDSSAGIG KSGAQPAKKR LNFGQTGDTE |
| 8 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPEPAD VEMIPQYGYL TLNDGSQAVG RSSFYCLEYY PSQMLRTGNN FQFSYEFENV |
| | | PFHSSYAHSQ SLDRLMNPLI DQYLYYLSKT INGSVQVGRTS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQID NO: 166) |
| AAV9.AAA. | SSGSQKF | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 9) | AKKRLLEPLG LVEEAAKTAP GKKRPVEQSP QEPDSSAGIG KSGAQPAKKR LNFGQTGDTE |
| 9 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSSGSQKF GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 167) |
| AAV9.AAA. | SGTVLRS | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 10) | AKKRLLEPLG LVEEAAKTAP GKKRPVEQSP QEPDSSAGIG KSGAQPAKKR LNFGQTGDTE |
| 10 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSGTVLRS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 168) |
| AAV9.AAA. | SHTKSAT | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 11) | AKKRLLEPLG LVEEAAKTAP GKKRPVEQSP QEPDSSAGIG KSGAQPAKKR LNFGQTGDTE |
| 11 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSHTKSAT GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 169) |
| AAV9.AAA. | DQARVRI | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 12) | AKKRLLEPLG LVEEAAKTAP GKKRPVEQSP QEPDSSAGIG KSGAQPAKKR LNFGQTGDTE |
| 12 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSDQARVRI GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 170) |
| AAV9.AAA. | RGAVQK | MAADGYLPDW LEDNLSEGIR EWWALKPGAP QPKANQQHQD NARGLVLPGY KYLGPGNGLD |
| VR4- | V | KGEPVNAADA AALEHDKAYD QQLKAGDNPY LKYNHADAEF QERLKEDTSE GGNLGRAVFQ |
| peptide | (SEQ ID | AKKRLLEPLG LVEEAAKTAP GKKRPVEQSP QEPDSSAGIG KSGAQPAKKR LNFGQTGDTE |
| 13 | NO: 13) | SVPDPQPIGE PPAAPSGVGS LTMASGGGAP VADNNEGADG VGSSSGNWHC DSQWLGDRVI |
| | and | TTSTRTWALP TYNNHLYKQI SNSTSGGSSN DNAYFGYSTP WGYFDFNRFH CHFSPRDWQR |
| | 496NNN498 | LINNNWGFRP KRLNFKLFNI QVKEVTDNNG VKTIANNLTS TVQVFTDSDY QLPYVLGSAH |
| | substitution | EGCLPPFPAD VFMIPQYGYL TLNDGSQAVG RSSFYCLEYF PSQMLRTGNN FQFSYEFENV |
| | to AAA | PFHSSYAHSQ SLDRLMNPLI DQYLYYLSKT INGSRGAVQKV GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 171) |
| AAV9.AAA. | VGRDSRS | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 14) | AKKRLLEPLG LVEEAAKTAP GKKRPVEQSP QEPDSSAGIG KSGAQPAKKR LNFGQTGDTE |
| 14 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVGRDSRS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 172) |
| AAV9.AAA. | SRSDRQF | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 15) | AKKRLLEPLG LVEEAAKTAP GKKRPVEQSP QEPDSSAGIG KSGAQPAKKR LNFGQTGDTE |
| 15 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSRSDRQF GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQCTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 173) |
| AAV9.AAA. | VTRSQIM | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 16) | AKKRLLEPLG LVEEAAKTAP GKKRPVEQSP QEPDSSAGIG KSGAQPAKKR LNFGQTGDTE |
| 16 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVTRSQIM GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 174) |
| AAV9.AAA. | QVGARH | MAADGYLPDW LEDNLSEGIR EWWALKPGAP QPKANQQHQD NARGLVLPGY KYLGPGNGLD |
| VR4- | P | KGEPVNAADA AALEHDKAYD QQLKAGDNPY LKYNHADAEF QERLKEDTSF GGNLGRAVFQ |
| peptide | (SEQ ID | AKKRLLEPLG LVEEAAKTAP GKKRPVEQSP QEPDSSAGIG KSGAQPAKKR LNFGQTGDTE |
| 17 | NO: 17) | SVPDPQPIGE PPAAPSGVGS LTMASGGGAP VADNNEGADG VGSSSGNWHC DSQWLGDRVI |
| | and | TTSTRTWALP TYNNHLYKQI SNSTSGGSSN DNAYFGYSTP WGYFDFNRFH CHFSPRDWQR |
| | 496NNN498 | LINNNWGFRP KRLNFKLFNI QVKEVTDNNG VKTIANNLTS TVQVFTDSDY QLPYVLGSAH |
| | substitution | EGCLPPFPAD VFMIPQYGYL TLNDGSQAVG RSSFYCLEYF PSQMLRTGNN FQFSYEFENV |
| | to AAA | PFHSSYAHSQ SLDRLMNPLI DQYLYYLSKT INGSQVGARHP GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 175) |
| AAV9.AAA. | GRAAASL | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 18) | AKKRLLEPLC LVEEAAKTAP GKKRPVEQSP QEPDSSAGIG KSGAQPAKKR LNFGQTGDTE |
| 18 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGRAAASL GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ IDNO: 176) |
| AAV9.AAA. | PSGVPIR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 19) | AKKRLLEPLG LVEEAAKTAP GKKRPVEQSP QEPDSSAGIG KSGAQPAKKR LNFGQTGDTE |
| 19 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPSGVPIR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 177) |
| AAV9.AAA. | MSYNQA | MAADGYLPDW LEDNLSEGIR EWWALKPGAP QPKANQQHQD NARGLVLPGY KYLGPGNGLD |
| VR4- | T | KGEPVNAADA AALEHDKAYD QQLKAGDNPY LKYNHADAEF QERLKEDTSF GGNLGRAVFQ |
| peptide | (SEQ ID | AKKRLLEPLG LVEEAAKTAP GKKRPVEQSP QEPDSSAGIG KSGAQPAKKR LNFGQTGDTE |
| 20 | NO: 20) | SVPDPQPIGE PPAAPSGVGS LTMASGGGAP VADNNEGADG VGSSSGNWHC DSQWLGDRVI |
| | and | TTSTRTWALP TYNNHLYKQI SNSTSGGSSN DNAYFGYSTP WGYFDFNRFH CHFSPRDWQR |
| | 496NNN498 | LINNNWGFRP KRLNFKLFNI QVKEVTDNNG VKTIANNLTS TVQVFTDSDY QLPYVLGSAH |
| | substitution | EGCLPPFPAD VFMIPQYGYL TLNDGSQAVG RSSFYCLEYF PSQMLRTGNN FQFSYEFENV |
| | to AAA | PFHSSYAHSQ SLDRLMNPLI DQYLYYLSKT INGSMSYNQAT GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPICTRYLT RNL (SEQ ID NO: 178) |
| AAV9.AAA. | ITGGVRV | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 21) | AKKRLLEPLG LVEEAAKTAP GKKRPVEQSP QEPDSSAGIG KSGAQPAKKR LNFGQTGDTE |
| 21 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSITGGVRV GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 179) |
| AAV9.AAA. | SRSDRQV | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 22) | AKKRLLEPLG LVEEAAKTAP GKKRPVEQSP QEPDSSAGIG KSGAQPAKKR LNFGQTGDTE |
| 22 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSRSDRQV GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 180) |
| AAV9.AAA. | TSGVPIR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 23) | AKKRLLEPLG LVEEAAKTAP GKKRPVEQSP QEPDSSAGIG KSGAQPAKKR LNFGQTGDTE |
| 23 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSTSGVPIR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 181) |
| AAV9.AAA. | AQVGRA | MAADGYLPDW LEDNLSEGIR EWWALKPGAP QPKANQQHQD NARGLVLPGY KYLGPGNGLD |
| VR4- | S | KGEPVNAADA AALEHDKAYD QQLKAGDNPY LKYNHADAEF QERLKEDTSF GGNLGRAVFQ |
| peptide | (SEQ ID | AKKRLLEPLG LVEEAAKTAP GKKRPVEQSP QEPDSSAGIG KSGAQPAKKR LNFGQTGDTE |
| 24 | NO: 24) | SVPDPQPIGE PPAAPSGVGS LTMASGGGAP VADNNEGADG VGSSSGNWHC DSQWLGDRVI |
| | and | TTSTRTWALP TYNNHLYKQI SNSTSGGSSN DNAYFGYSTP WGYFDFNRFH CHFSPRDWQR |
| | 496NNN498 | LINNNWGFRP KRLNFKLFNI QVKEVTDNNG VKTIANNLTS TVQVFTDSDY QLPYVLGSAH |
| | substitution | EGCLPPFPAD VFMIPQYGYL TLNDGSQAVG RSSFYCLEYF PSQMLRTGNN FQFSYEFENV |
| | to AAA | PFHSSYAHSQ SLDRLMNPLI DQYLYYLSKT INGSAQVGRAS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ IDNO: 182) |
| AAV9.AAA. | VKASHHS | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 25) | AKKRLLEPLG LVEEAAKTAP GKKRPVEQSP QEPDSSAGIG KSGAQPAKKR LNFGQTGDTE |
| 25 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVKASHHS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTCQVSVEIEWELQKENSK RWNPEIQYTS NYYKSNNVEF |
| | | AVNTEGVYSE PRPIGTRYLT RNL (SEQ IDNO: 183) |
| AAV9.AAA. | SHTKTVT | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 26) | AKKRLLEPLG LVEEAAKTAP GKKRPVEQSP QEPDSSAGIG KSGAQPAKKR LNFGQTGDTE |
| 26 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSHTKTVT GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAEN KDKLNSEITQ YSTGQVSVET EWELQKENSK RWNPELQYTS NYYKSNNVEF |
| | | AVNTEGVYSE PRPIGTRYLT RNL (SEQ IDNO: 184) |
| AAV9.AAA. | GSVQTVR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 27) | AKKRLLEPLG LVEEAAKTAP GKKRPVEQSP QEPDSSAGIG KSGAQPAKKR LNFGQTGDTE |
| 27 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGSVQTVR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 185) |
| AAV9.AAA. | DTRIRSM | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 28) | AKKRLLEPLG LVEEAAKTAP GKKRPVEQSP QEPDSSAGIG KSGAQPAKKR LNFGQTGDTE |
| 28 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSDTRIRSM GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 186) |
| AAV9.AAA. | SRTKTAT | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 29) | AKKRLLEPLG LVEEAAKTAP GKKRPVEQSP QEPDSSAGIG KSGAQPAKKR LNFGQTGDTE |
| 29 | and | SVPDPQPIGE PPAAPSGVGS LTMASGGGAP VADNNEGADG VGSSSGNWHC DSQWLGDRVI |
| | 496NNN498 | ITSTRTWALP TYNNHLYKQI SNSTSGGSSN DNAYFGYSTP WGYFDFNREH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSRTKTAT GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 187) |
| AAV9.AAA. | QVGARQ | MAADGYLPDW LEDNLSEGIR EWWALKPGAP QPKANQQHQD NARGLVLPGY KYLGPGNGLD |
| VR4- | S | KGEPVNAADA AALEHDKAYD QQLKAGDNPY LKYNHADAEF QERLKEDTSF GGNLGRAVFQ |
| peptide | (SEQ ID | AKKRLLEPLG LVEEAAKTAP GKKRPVEQSP QEPDSSAGIG KSGAQPAKKR LNFGQTGDTE |
| 30 | NO: 30) | SVPDPQPIGE PPAAPSGVGS LTMASGGGAP VADNNEGADG VGSSSGNWHC DSQWLGDRVI |
| | and | TTSTRTWALP TYNNHLYKQI SNSTSGGSSN DNAYFGYSTP WGYFDFNRFH CHFSPRDWQR |
| | 496NNN498 | LINNNWGFRP KRLNFKLFNI QVKEVTDNNG VKTIANNLTS TVQVFTDSDY QLPYVLGSAH |
| | substitution | EGCLPPFPAD VFMIPQYGYL TLNDGSQAVG RSSFYCLEYF PSQMLRTGNN FQFSYEFENV |
| | to AAA | PFHSSYAHSQ SLDRLMNPLI DQYLYYLSKT INGSQVGARQS GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPçILIKNTP |
| | | VPADPPTAEN KDKLNSFITQ YSTGQVSVEL EWELQKENSK RWNPELQYTS NYYKSNNVEF |
| | | AVNTEGVYSE PRPIGTRYLT RNL (SEQ ID NO: 188) |
| AAV9.AAA. | SVTSVRV | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 37) | AKKRLLEPLG LVEEAAKTAP GKKRPVEQSP QEPDSSAGIG KSGAQPAKKR LNFGQTGDTE |
| 37 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSVTSVRV GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 189) |
| AAV9.AAA. | MKYRGT | MAADGYLPDW LEDNLSEGIR EWWALKPGAP QPKANQQHQD NARGLVLPGY KYLGPGNGLD |
| VR4- | D (SEQ ID | KGEPVNAADA AALEHDKAYD QQLKAGDNPY LKYNHADAEF QERLKEDTSF GGNLGRAVFQ |
| peptide | NO: 45) | AKKRLLEPLG LVEEAAKTAP GKKRPVEQSP QEPDSSAGIG KSGAQPAKKR LNFGQTGDTE |
| 45 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSMKYRGTD GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 190) |
| AAV9.AAA. | SIAKNSA | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 76) | AKKRLLEPLG LVEEAAKTAP GKKRPVEQSP QEPDSSAGIG KSGAQPAKKR LNFGQTGDTE |
| 76 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSIAKNSA GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPçILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 191) |
| AAV9.AAA. | HMGQYG | MAADGYLPDW LEDNLSEGIR EWWALKPGAP QPKANQQHQD NARGLVLPGY KYLGPGNGLD |
| VR4- | K (SEQ ID | KGEPVNAADA AALEHDKAYD QQLKAGDNPY LKYNHADAEF QERLKEDTSF GGNLGRAVFQ |
| peptide | NO:82) | AKKRLLEPLG LVEEAAKTAP GKKRPVEQSP QEPDSSAGIG KSGAQPAKKR LNFGQTGDTE |
| 82 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSHMGQYGK GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 192) |
| AAV9.AAA. | MRAVSQ | MAADGYLPDW LEDNLSEGIR EWWALKPGAP QPKANQQHQD NARGLVLPGY KYLGPGNGLD |
| VR4- | L (SEQ ID | KGEPVNAADA AALEHDKAYD QQLKAGDNPY LKYNHADAEF QERLKEDTSF GGNLGRAVFQ |
| peptide | NO: 109) | AKKRLLEPLG LVEEAAKTAP GKKRPVEQSP QEPDSSAGIG KSGAQPAKKR LNFGQTGDTE |
| 109 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSMRAVSQL GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 193) |
| AAV9.AAA. | PRQYVPG | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 110) | AKKRLLEPLG LVEEAAKTAP GKKRPVEQSP QEPDSSAGIG KSGAQPAKKR LNFGQTGDTE |
| 110 | and | SVPDPQPIGE PPAAPSGVGS LTMASGGGAP VADNNEGADG VCSSSCNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPRQYVPG GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 194) |
| AAV9.AAA. | RSSSGR | MAADGYLPDW LEDNLSEGIR EWWALKPCAP QPKANQQHQD NARGLVLPCY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 111) | AKKRLLEPLG LVEEAAKTAP GKKRPVEQSP QEPDSSAGIG KSGAQPAKKR LNFGQTGDTE |
| 111 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSRSSSGR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | CWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 195) |
| AAV9.AAA. | VVKSTKS | MAADGYLPDW LEDNLSEGIR EWWALKPGAP QPKANQQHQD NARGLVLPGY KYLGPGNGLD |
| VR4- | (SEQ ID | AKGEPVNAADA ALEHDKAYD QQLKAGDNPY LKYNHADAEF QERLKEDTSF GGNLGRAVFQ |
| peptide | NO: 112) | AKKRLLEPLG LVEEAAKTAP GKKRPVEQSP QEPDSSAGIG KSGAQPAKKR LNFGQTGDTE |
| 112 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVVKSTKS CQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 196) |
| AAV9.AAA. | PSSVQHR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 118) | AKKRLLEPLG LVEEAAKTAP GKKRPVEQSP QEPDSSAGIG KSGAQPAKKR LNFGQTGDTE |
| 118 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPSSVQHR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 197) |
| AAV9.AAA. | SSSVQHR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 119) | AKKRLLEPLG LVEEAAKTAP GKKRPVEQSP QEPDSSAGIG KSGAQPAKKR LNFGQTGDTE |
| 119 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSSSVQHR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGVSVEI EWELQKENSK RWNPEIQYTS NYYKSNNVEF |
| | | AVNTEGVYSE PRPIGTRYLT RNL (SEQ ID NO: 198) |
| AAV9.AAA. | SGMQER | MAADGYLPDW LEDNLSEGIR EWWALKPGAP QPKANQQHQD NARGLVLPGY KYLGPGNGLD |
| VR4- | R (SEQ ID | KGEPVNAADA AALEHDKAYD QQLKAGDNPY LKYNHADAEF QERLKEDTSF GGNLGRAVFQ |
| peptide | NO: 120) | AKKRLLEPLC LVEEAAKTAP CKKRPVEQSP QEPDSSAGIG KSGAQPAKKR LNFGQTGDTE |
| 120 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSGMQERR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 199) |
| AAV9.AAA. | LERGNLE | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 121) | AKKRLLEPLG LVEEAAKTAP GKKRPVEQSP QEPDSSAGIG KSGAQPAKKR LNFGQTGDTE |
| 121 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSLERGNLE GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF CKQCTGRDNV DADKVMITNE EEIKTTNPVA TESYCQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 200) |
| AAV9.AAA. | YRDQRV | MAADGYLPDW LEDNLSEGIR EWWALKPGAP QPKANQQHQD NARGLVLPGY KYLGPGNGLD |
| VR4- | T (SEQ ID | KGEPVNAADA AALEHDKAYD QQLKAGDNPY LKYNHADAEF QERLKEDTSF GGNLGRAVFQ |
| peptide | NO: 122) | AKKRLLEPLG LVEEAAKTAP GKKRPVEQSP QEPDSSAGIG KSGAQPAKKR LNFGQTGDTE |
| 122 | and | SVPDPQPIGE PPAAPSGVGS LTMASGGGAP VADNNEGADG VGSSSGNWHC DSQWLGDRVI |
| | 496NNN498 | ITSTRTWALP TYNNHLYKQL SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSYRDQRVT GQNQQTLKFSVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 201) |
| AAV9.AAA. | PSAVQHR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 123) | AKKRLLEPLG LVEEAAKTAP GKKRPVEQSP QEPDSSAGIG KSGAQPAKKR LNFGQTGDTE |
| 123 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPSAVQHR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAEN KDKLNSFITQ YSTGQVSVEL EWELQKENSK RWNPELQYTS NYYKSNNVEF |
| | | AVNTEGVYSE PRPIGTRYLT RNL (SEQ ID NO: 202) |
| AAV9.AAA. | YVGGRA | MAADGYLPDW LEDNLSEGIR EWWALKPGAP QPKANQQHQD NARGLVLPGY KYLGPGNGLD |
| VR4- | V (SEQ ID | KGEPVNAADA AALEHDKAYD QQLKAGDNPY LKYNHADAEF QERLKEDTSF GGNLGRAVFQ |
| peptide | NO: 124) | AKKRLLEPLG LVEEAAKTAP GKKRPVEQSP QEPDSSAGIG KSGAQPAKKR LNFGQTGDTE |
| 124 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSYVGGRAV GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 203) |
| AAV9.AAA. | IGSRGVA | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 125) | AKKRLLEPLG LVEEAAKTAP GKKRPVEQSP QEPDSSAGIG KSGAQPAKKR LNFGQTGDTE |
| 125 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSIGSRGVA GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 204) |
| AAV9.AAA. | SDVSRPR | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 126) | AKKRLLEPLG LVEEAAKTAP GKKRPVEQSP QEPDSSAGIG KSGAQPAKKR LNFGQTGDTE |
| 126 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSDVSRPR GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 205) |
| AAV9.AAA. | GSVRQA | MAADGYLPDW LEDNLSEGIR EWWALKPGAP QPKANQQHQD NARGLVLPGY KYLGPGNGLD |
| VR4- | A (SEQ ID | KGEPVNAADA AALEHDKAYD QQLKAGDNPY LKYNHADAEF QERLKEDTSF GGNLGRAVFQ |
| peptide | NO: 127) | AKKRLLEPLG LVEEAAKTAP GKKRPVEQSP QEPDSSAGIG KSGAQPAKKR LNFGQTGDTE |
| 127 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGSVRQAA GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 206) |
| AAV9.AAA. | QSPHTSQ | MAADGYLPDW LEDNLSEGIR EWWALKPGAP QPKANQQHQD NARGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 128) | AKKRLLEPLG LVEEAAKTAP GKKRPVEQSP QEPDSSAGIG KSGAQPAKKR LNFGQTGDTE |
| 128 | and | SVPDPQPIGE PPAAPSGVGS LTMASGGGAP VADNNEGADG VGSSSGNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSQSPHTSQ GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 207) |
| AAV9.AAA. | ASQAYH | MAADGYLPDW LEDNLSEGIR EWWALKPGAP QPKANQQHQD NARGLVLPGY KYLGPGNGLD |
| VR4- | G (SEQ ID | KGEPVNAADA AALEHDKAYD QQLKAGDNPY LKYNHADAEF QERLKEDTSF GGNLGRAVFQ |
| peptide | NO: 129) | AKKRLLEPLG LVEEAAKTAP GKKRPVEQSP QEPDSSAGIG KSGAQPAKKR LNFGQTGDTE |
| 129 | and | SVPDPQPIGE PPAAPSCVGS LTMASGGGAP VADNNEGADC VCSSSCNWHC DSQWLGDRVI |
| | 496NNN498 | TTSTRTWALP TYNNHLYKQI SNSTSGGSSN DNAYFGYSTP WGYFDFNRFH CHFSPRDWQR |
| | substitution | LINNNWGFRP KRLNFKLFNI QVKEVTDNNG VKTIANNLTS TVQVFTDSDY QLPYVLGSAH |
| | to AAA | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSASQAYHG GQNQQTLKF SVAGPSNMAV |
| | | QGRNYIPGPS YRQQRVSTTV TQAAASEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 208) |
| AAV9.AAA. | X₁-Q-V- | MAADGYLPDW LEDNLSEGIR EWWALKPGAP QPKANQQHQD NARGLVLPCY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGAQPAKKR LNFGQTGDTE |
| 135 | ID NO: | SVPDPQPIGE PPAAPSGVGS LTMASGGGAP VADNNEGADG VGSSSGNWHC DSQWLGDRVI |
| | 135) | TTSTRTWALP TYNNHLYKQI SNSTSGGSSN DNAYFGYSTP WGYFDFNRFH CHFSPRDWQR |
| | and | LINNNWGFRP KRLNFKLFNI QVKEVTDNNG VKTIANNLTS TVQVFTDSDY QLPYVLGSAH |
| | 496NNN498 | EGCLPPFPAD VFMIPQYGYL TLNDGSQAVG RSSFYCLEYF PSQMLRTGNN FQFSYEFENV |
| | substitution | PFHSSYAHSQ SLDRLMNPLI DQYLYYLSKT INGSX₁QVX₂X₃X₄X₅ GQNQQTLKF |
| | to AAA | SVAGPSNMAV QGRNYIPGPS YRQQRVSTTV TQAAASEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT CWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRYLT RNL (SEQ ID NO: 209) |
| | | wherein X_{1 is} V or A, X_{2 is} S, G, V or A, X_{3 is} R or H, X_{4 is} any |
| | | amino acid and X_{5 is} S, G, V or A |
| AAV9.AAA. | X₁-Q-V- | MAADGYLPDW LEDNLSEGIR EWWALKPGAP QPKANQQHQD NARGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGAQPAKKR LNFGQTGDTE |
| 136 | ID NO: | SVPDPQPIGE PPAAPSGVGS LTMASGGGAP VADNNEGADG VGSSSGNWHC DSQWLGDRVI |
| | 136) | TTSTRTWALP TYNNHLYKQI SNSTSGGSSN DNAYFGYSTP WGYFDFNRFH CHFSPRDWQR |
| | and | LINNNWGFRP KRLNFKLFNI QVKEVTDNNG VKTIANNLTS TVQVFTDSDY QLPYVLGSAH |
| | 496NNN498 | EGCLPPFPAD VFMIPQYGYL TLNDGSQAVG RSSFYCLEYF PSQMLRTGNN FQFSYEFENV |
| | substitution | PFHSSYAHSQ SLDRLMNPLI DQYLYYLSKT INGSX₁QVX₂X₃X₄X₅ GQNQQTLKF |
| | to AAA | SVAGPSNMAV QGRNYIPGPS YRQQRVSTTV TQAAASEFAW PGASSWALNG RNSLMNPGPA |
| | | MASHKEGEDR FFPLSGSLIF DADKVMITNE GKQGTGRDNV EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRYST RNL (SEQ ID NO: 210) |
| | | wherein X_{1 is} V or A, X_{2 is} S, G, V or A, X_{3 is} R or H, X_{4 is} any |
| | | amino acid and X_{5 is} S or A |
| AAV9.AAA. | X₁-Q-V- | MAADGYLPDW LEDNLSEGIR EWWALKPGAP QPKANQQHQD NARGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGAQPAKKR LNFGQTGDTE |
| 137 | ID NO: | SVPDPQPIGE PPAAPSGVGS LTMASGGGAP VADNNEGADG VGSSSGNWHC DSQWLGDRVI |
| | 137) | TTSTRTWALP TYNNHLYKQI SNSTSGGSSN DNAYFGYSTP WGYFDFNRFH CHFSPRDWQR |
| | and | LINNNWGFRP KRLNFKLFNI QVKEVTDNNG VKTIANNLTS TVQVFTDSDY QLPYVLGSAH |
| | 496NNN498 | EGCLPPFPAD VFMIPQYGYL TLNDGSQAVG RSSFYCLEYF PSQMLRTGNN FQFSYEFENV |
| | substitution | PFHSSYAHSQ SLDRLMNPLI DQYLYYLSKT INGSX₁QVX₂X₃X₄X₅ GQNQQTLKF |
| | to AAA | SVAGPSNMAV QGRNYIPGPS YRQQRVSTTV TQAAASEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRYST RNL (SEQ ID NO: 211) |
| | | wherein X_{1 is} V or A, X_{2 is} S, G, V or A, X_{3 is} R or H, X_{4 is} T, S, V, |
| | | Y, A or P and X_{5 is} S, G, V or A |
| AAV9.AAA. | X₁-Q-V- | MAADGYLPDW LEDNLSEGIR EWWALKPGAP QPKANQQHQD NARGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGAQPAKKR LNFGQTGDTE |
| 138 | ID NO: | SVPDPQPIGE PPAAPSGVGS LTMASGGGAP VADNNEGADG VGSSSGNWHC DSQWLGDRVI |
| | 138) | TTSTRTWALP TYNNHLYKQI SNSTSGGSSN DNAYFGYSTP WGYFDFNRFH CHFSPRDWQR |
| | and | LINNNWGFRP KRLNFKLFNI QVKEVTDNNG VKTIANNLTS TVQVFTDSDY QLPYVLGSAH |
| | 496NNN498 | EGCLPPFPAD VFMIPQYGYL TLNDGSQAVG RSSFYCLEYF PSQMLRTGNN FQFSYEFENV |
| | substitution | PFHSSYAHSQ SLDRLMNPLI DQYLYYLSKT INGSX₁QVX₂X₃X₄X₅ GQNQQTLKF |
| | to AAA | SVAGPSNMAV QGRNYIPGPS YRQQRVSTTV TQAAASEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRY_T RNL (SEQ ID NO: 212) |
| | | wherein X_{1 is} V or A, X_{2 is} S, G, V or A, X_{3 is} R or H, X_{4 is} T, S, |
| | | V, Y, A or P and X_{5 is} S or A |
| AAV9.VR4- | SGTVIRS | MAADGYLPDW LEDNLSEGIR EWWALKPGAP QPKANQQHQD NARGLVLPGY KYLGPGNGLD |
| peptide 1 | (SEQ ID | KGEPVNAADA AALEHDKAYD QQLKAGDNPY LKYNHADAEF QERLKEDTSF GGNLGRAVFQ |
| | NO: 1) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSGTVIRS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 213) |
| AAV9.VR4- | GRPAP | MAADGYLPDW LEDNLSEGIR EWWALKPGAP QPKANQQHQD NARGLVLPGY KYLGPGNGLD |
| peptide 2 | (SEQ ID | KGEPVNAADA AALEHDKAYD QQLKAGDNPY LKYNHADAEF QERLKEDTSF GGNLGRAVFQ |
| | NO: 2) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGRPAP GQNQQTLKF SVAGPSNMAV |
| | | QGRNYIPGPS YRQQRVSTTV TQNNNSEFAW PCASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAEN KDKLNSFITQ YSTGQVSVEL EWELQKENSK RWNPELQYTS NYYKSNNVEF |
| | | AVNTEGVYSE PRPIGTRYLT RNL (SEQ ID NO: 214) |
| AAV9.VR4- | KDQKIGP | MAADGYLPDW LEDNLSEGIR EWWALKPGAP QPKANQQHQD NARGLVLPGY KYLGPGNGLD |
| peptide 3 | (SEQ ID | KGEPVNAADA AALEHDKAYD QQLKAGDNPY LKYNHADAEF QERLKEDTSF GGNLGRAVFQ |
| | NO: 3) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSKDQKIGP GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 215) |
| AAV9.VR4- | TRQYVPG | MAADGYLPDW LEDNLSEGIR EWWALKPGAP QPKANQQHQD NARGLVLPGY KYLGPGNGLD |
| peptide 4 | (SEQ ID | KGEPVNAADA AALEHDKAYD QQLKAGDNPY LKYNHADAEF QERLKEDTSF GGNLGRAVFQ |
| | NO: 4) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSTRQYVPG GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 216) |
| AAV9.VR4- | VQVGRA | MAADGYLPDW LEDNLSEGIR EWWALKPGAP QPKANQQHQD NARGLVLPGY KYLGPGNGLD |
| peptide 5 | A | KGEPVNAADA AALEHDKAYD QQLKAGDNPY LKYNHADAEF QERLKEDTSF GGNLGRAVFQ |
| | (SEQ ID | AKKRLLEPLG LVEEAAKTAP GKKRPVEQSP QEPDSSAGIG KSGAQPAKKR LNFGQTGDTE |
| | NO: 5) | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVQVGRAA GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 217) |
| AAV9.VR4- | FTQSRGG | MAADGYLPDW LEDNLSEGIR EWWALKPGAP QPKANQQHQD NARGLVLPGY KYLGPGNGLD |
| peptide 6 | (SEQ ID | KGEPVNAADA AALEHDKAYD QQLKAGDNPY LKYNHADAEF QERLKEDTSF GGNLGRAVFQ |
| | NO: 6) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSFTQSRGG GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 218) |
| AAV9.VR4- | SRSDRPA | MAADGYLPDW LEDNLSEGIR EWWALKPGAP QPKANQQHQD NARGLVLPGY KYLGPGNGLD |
| peptide 7 | (SEQ ID | KGEPVNAADA AALEHDKAYD QQLKAGDNPY LKYNHADAEF QERLKEDTSF GGNLGRAVFQ |
| | NO: 7) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSRSDRPA GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 219) |
| AAV9.VR4- | VQVGRTS | MAADGYLPDW LEDNLSEGIR EWWALKPGAP QPKANQQHQD NARGLVLPGY KYLGPGNGLD |
| peptide 8 | (SEQ ID | KGEPVNAADA AALEHDKAYD QQLKAGDNPY LKYNHADAEF QERLKEDTSF GGNLGRAVFQ |
| | NO: 8) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VCSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVQVGRTS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 220) |
| AAV9.VR4- | SSGSQKF | MAADGYLPDW LEDNLSEGIR EWWALKPCAP QPKANQQHQD NARGLVLPCY KYLGPGNGLD |
| peptide 9 | (SEQ ID | KGEPVNAADA AALEHDKAYD QQLKAGDNPY LKYNHADAEF QERLKEDTSF GGNLGRAVFQ |
| | NO: 9) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSSGSQKE GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | CWVQNQCILP CMVWQDRDVY LQGPIWAKIP HTDGNFHPSP LMGGFCMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 221) |
| AAV9.VR4- | SGTVLRS | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 10 | NO: 10) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSGTVLRS CQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHPPPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 222) |
| AAV9.VR4- | SHTKSAT | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 11 | NO: 11) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSHTKSAT GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 223) |
| AAV9.VR4- | DQARVRI | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 12 | NO: 12) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSDQARVRI GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 224) |
| AAV9.VR4- | RGAVQK | MAADGYLPDW LEDNLSEGIR EWWALKPGAP QPKANQQHQD NARGLVLPGY KYLGPGNGLD |
| peptide | V | KGEPVNAADA AALEHDKAYD QQLKAGDNPY LKYNHADAEF QERLKEDTSF GGNLGRAVFQ |
| 13 | (SEQ ID | AKKRLLEPLG LVEEAAKTAP GKKRPVEQSP QEPDSSAGIG KSGAQPAKKR LNFCQTGDTE |
| | NO: 13) | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSRGAVQKV GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 225) |
| AAV9.VR4- | VGRDSRS | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 14 | NO: 14) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVGRDSRS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF CKQCTGRDNV DADKVMITNE EEIKTTNPVA TESYCQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 226) |
| AAV9.VR4- | SRSDRQF | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 15 | NO: 15) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | ECCLPPFPAD VFMIPQYCYL TLNDCSQAVG RSSFYCLEYF PSQMLRTCNN FQFSYEFENV |
| | | PFHSSYAHSQ SLDRLMNPLI DQYLYYLSKT INGSSRSDRQF GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 227) |
| AAV9.VR4- | VTRSQIM | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 16 | NO: 16) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP VFMIPQYGYL TLNDGSQAVG RSSFYCLEYF PSQMLRTGNN FQFSYEFENV |
| | | EGCLPPFPAD KRLNFKLFNI QVKEVTDNNG VKTIANNLTS TVQVFTDSDY QLPYVLGSAH |
| | | PFHSSYAHSQ SLDRLMNPLI DQYLYYLSKT INGSVTRSQIM GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 228) |
| AAV9.VR4- | QVGARH | MAADGYLPDW LEDNLSEGIR EWWALKPGAP QPKANQQHQD NARGLVLPGY KYLGPGNGLD |
| peptide | P | KGEPVNAADA AALEHDKAYD QQLKAGDNPY LKYNHADAEF QERLKEDTSF GGNLGRAVFQ |
| 17 | (SEQ ID | AKKRLLEPLG LVEEAAKTAP GKKRPVEQSP QEPDSSAGIG KSGAQPAKKR LNFGQTGDTE |
| | NO: 17) | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSQVGARHP GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 229) |
| AAV9.VR4- | GRAAASL | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 18 | NO: 18) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGRAAASL GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 230) |
| AAV9.VR4- | PSGVPIR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSE GGNLGRAVFQ |
| 19 | NO: 19) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPSGVPIR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 231) |
| AAV9.VR4- | MSYNQA | MAADGYLPDW LEDNLSEGIR EWWALKPGAP QPKANQQHQD NARGLVLPGY KYLGPGNGLD |
| peptide | T | KGEPVNAADA AALEHDKAYD QQLKAGDNPY LKYNHADAEF QERLKEDTSF GGNLGRAVFQ |
| 20 | (SEQ ID | AKKRLLEPLG LVEEAAKTAP GKKRPVEQSP QEPDSSAGIG KSGAQPAKKR LNFGQTGDTE |
| | NO: 20) | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSMSYNQAT GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 232) |
| AAV9.VR4- | ITGGVRV | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 21 | NO: 21) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSITGGVRV CQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 233) |
| AAV9.VR4- | SRSDRQV | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 22 | NO: 22) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSRSDRQV GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 234) |
| AAV9.VR4- | TSGVPIR | MAADGYLPDW LEDNLSEGIR EWWALKPGAP QPKANQQHQD NARGLVLPCY KYLGPGNGLD |
| peptide | (SEQ ID | KGEPVNAADA AALEHDKAYD QQLKAGDNPY LKYNHADAEF QERLKEDTSF GGNLGRAVFQ |
| 23 | NO: 23) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSTSGVPIR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 235) |
| AAV9.VR4- | AQVGRA | MAADGYLPDW LEDNLSEGIR EWWALKPGAP QPKANQQHQD NARGLVLPGY KYLGPGNGLD |
| peptide | S | KGEPVNAADA AALEHDKAYD QQLKAGDNPY LKYNHADAEF QERLKEDTSF GGNLGRAVFQ |
| 24 | (SEQ ID | AKKRLLEPLG LVEEAAKTAP GKKRPVEQSP QEPDSSAGIG KSGAQPAKKR LNFGQTGDTE |
| | NO: 24) | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSAQVGRAS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 236) |
| AAV9.VR4- | VKASHHS | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 25 | NO: 25) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WCYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVKASHHS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 237) |
| AAV9.VR4- | SHTKTVT | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 26 | NO: 26) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSHTKTVT GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 238) |
| AAV9.VR4- | GSVQTVR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 27 | NO: 27) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGSVQTVR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 239) |
| AAV9.VR4- | DTRIRSM | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 28 | NO: 28) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSDTRIRSM GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 240) |
| AAV9.VR4- | SRTKTAT | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 29 | NO: 29) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | ECCLPPFPAD VFMIPQYCYL TLNDCSQAVG RSSFYCLEYF PSQMLRTCNN FQFSYEFENV |
| | | PFHSSYAHSQ SLDRLMNPLI DQYLYYLSKT INGSSRTKTAT GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 241) |
| AAV9.VR4- | QVGARQ | MAADGYLPDW LEDNLSEGIR EWWALKPGAP QPKANQQHQD NARGLVLPGY KYLGPGNGLD |
| peptide | S | KGEPVNAADA AALEHDKAYD QQLKAGDNPY LKYNHADAEF QERLKEDTSF GGNLGRAVFQ |
| 30 | (SEQ ID | AKKRLLEPLG LVEEAAKTAP GKKRPVEQSP QEPDSSAGIG KSGAQPAKKR LNFGQTGDTE |
| | NO: 30) | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPEPAD VEMIPQYGYL TLNDGSQAVG RSSFYCLEYF PSQMLRTGNN FQFSYEFENV |
| | | PFHSSYAHSQ SLDRLMNPLI DQYLYYLSKT INGSQVGARQS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAEN KDKLNSEITQ YSTGQVSVEL EWELQKENSK RWNPELQYTS NYYKSNNVEF |
| | | AVNTEGVYSE PRPIGTRYLT RNL (SEQ ID NO: 242) |
| AAV9.VR4- | SVTSVRV | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 37 | NO: 37) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSVTSVRV GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 243) |
| AAV9.VR4- | MKYRGT | MAADGYLPDW LEDNLSEGIR EWWALKPGAP QPKANQQHQD NARGLVLPGY KYLGPGNGLD |
| peptide | D (SEQ ID | KGEPVNAADA AALEHDKAYD QQLKAGDNPY LKYNHADAEF QERLKEDTSF GGNLGRAVFQ |
| 45 | NO: 45) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSMKYRGTD GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 244) |
| AAV9.VR4- | SIAKNSA | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSE GGNLGRAVFQ |
| 76 | NO: 76) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSIAKNSA GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 245) |
| AAV9.VR4- | HMGQYG | MAADGYLPDW LEDNLSEGIR EWWALKPGAP QPKANQQHQD NARGLVLPGY KYLGPGNGLD |
| peptide | K (SEQ ID | KGEPVNAADA AALEHDKAYD QQLKAGDNPY LKYNHADAEF QERLKEDTSF GGNLGRAVFQ |
| 82 | NO: 82) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSHMGQYGK GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 246) |
| AAV9.VR4- | MRAVSQ | MAADGYLPDW LEDNLSEGIR EWWALKPGAP QPKANQQHQD NARGLVLPGY KYLGPGNGLD |
| peptide | L (SEQ ID | KGEPVNAADA AALEHDKAYD QQLKAGDNPY LKYNHADAEF QERLKEDTSF GGNLGRAVFQ |
| 109 | NO: 109) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGERP KRLNFKLFNI QVKEVTDNNG VKTIANNLTS TVQVETDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSMRAVSQL CQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNEHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 247) |
| AAV9.VR4- | PRQYVPG | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 110 | NO: 110) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PEHSSYAHSQ SLDRLMNPLI DQYLYYLSKT INGSPRQYVPG GQNQQTLKE SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 248) |
| AAV9.VR4- | RSSSGR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 111 | NO: 111) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSRSSSGR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 249) |
| AAV9.VR4- | VVKSTKS | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 112 | NO: 112) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSVVKSTKS GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 250) |
| AAV9.VR4- | PSSVQHR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 118 | NO: 118) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WCYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPSSVQHR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 251) |
| AAV9.VR4- | SSSVQHR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 119 | NO: 119) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSSSVQHR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 252) |
| AAV9.VR4- | SGMQER | MAADGYLPDW LEDNLSEGIR EWWALKPGAP QPKANQQHQD NARGLVLPGY KYLGPGNGLD |
| peptide | R (SEQ ID | KGEPVNAADA AALEHDKAYD QQLKAGDNPY LKYNHADAEF QERLKEDTSF GGNLGRAVFQ |
| 120 | NO: 120) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDYQLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSGMQERR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAEN KDKLNSFITQ YSTGQVSVEL EWELQKENSK RWNPELQYTS NYYKSNNVEF |
| | | AVNTEGVYSE PRPIGTRYLT RNL (SEQ ID NO: 253) |
| AAV9.VR4- | LERGNLE | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 121 | NO: 121) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSLERGNLE GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 254) |
| AAV9.VR4- | YRDQRV | MAADGYLPDW LEDNLSEGIR EWWALKPGAP QPKANQQHQD NARGLVLPGY KYLGPGNGLD |
| peptide | T (SEQ ID | KGEPVNAADA AALEHDKAYD QQLKAGDNPY LKYNHADAEF QERLKEDTSF GGNLGRAVFQ |
| 122 | NO: 122) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSYRDQRVT GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 255) |
| AAV9.VR4- | PSAVQHR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 123 | NO: 123) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSPSAVQHR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHPPPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 256) |
| AAV9.VR4- | YVGGRA | MAADGYLPDW LEDNLSEGIR EWWALKPGAP QPKANQQHQD NARGLVLPGY KYLGPGNGLD |
| peptide | V (SEQ ID | KGEPVNAADA AALEHDKAYD QQLKAGDNPY LKYNHADAEF QERLKEDTSF GGNLGRAVFQ |
| 124 | NO: 124) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSYVGGRAV GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 257) |
| AAV9.VR4- | IGSRGVA | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 125 | NO: 125) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSIGSRGVA GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 258) |
| AAV9.VR4- | SDVSRPR | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 126 | NO: 126) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VCSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSSDVSRPR GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 258) |
| AAV9.VR4- | GSVRQA | MAADCYLPDW LEDNLSEGIR EWWALKPCAP QPKANQQHQD NARGLVLPCY KYLGPGNGLD |
| peptide | A (SEQ ID | KGEPVNAADA AALEHDKAYD QQLKAGDNPY LKYNHADAEF QERLKEDTSF GGNLGRAVFQ |
| 127 | NO: 127) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSGSVRQAA GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | CWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 260) |
| AAV9.VR4- | QSPHTSQ | MAADGYLPDW LEDNLSEGIR EWWALKPGAP QPKANQQHQD NARGLVLPGY KYLGPGNGLD |
| peptide | (SEQ ID | KGEPVNAADA AALEHDKAYD QQLKAGDNPY LKYNHADAEF QERLKEDTSF GGNLGRAVFQ |
| 128 | NO: 128) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSQSPHTSQ CQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 261) |
| AAV9.VR4- | ASQAYH | MAADGYLPDW LEDNLSEGIR EWWALKPGAP QPKANQQHQD NARGLVLPGY KYLGPGNGLD |
| peptide | G (SEQ ID | KGEPVNAADA AALEHDKAYD QQLKAGDNPY LKYNHADAEF QERLKEDTSF GGNLGRAVFQ |
| 129 | NO: 129) | AKKRLLEPLG LVEEAAKTAP GKKRPVEQSP QEPDSSAGIG KSGAQPAKKR LNFGQTGDTE |
| | | SVPDPQPIGE PPAAPSGVGS LTMASGGGAP VADNNEGADG VGSSSGNWHC DSQWLGDRVI |
| | | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSASQAYHG GQNQQTLKF SVAGPSNMAV |
| | | QGRNYIPGPS YRQQRVSTTV TQNNNSEFAW PGASSWALNG RNSLMNPGPA MASHKEGEDR |
| | | FFPLSGSLIF GKQGTGRDNV DADKVMITNE EEIKTTNPVA TESYGQVATN HQSAQAQAQT |
| | | GWVQNQGILP GMVWQDRDVY LQGPIWAKIP HTDGNFHPSP LMGGFGMKHP PPQILIKNTP |
| | | VPADPPTAFN KDKLNSFITQ YSTGQVSVEI EWELQKENSK RWNPEIQYTS NYYKSNNVEF |
| | | AVNTEGVYSE PRPIGTRYLT RNL (SEQ ID NO: 262) |
| AAV9.VR4- | X₁-Q-V- | MAADGYLPDW LEDNLSEGIR EWWALKPGAP QPKANQQHQD NARGLVLPGY KYLGPGNGLD |
| peptide | X₂-X₃-X₄- | KGEPVNAADA AALEHDKAYD QQLKAGDNPY LKYNHADAEF QERLKEDTSF GGNLGRAVFQ |
| 135 | X₅, (SEQ | AKKRLLEPLG LVEEAAKTAP GKKRPVEQSP QEPDSSAGIG KSGAQPAKKR LNFGQTGDTE |
| | ID NO: | SVPDPQPIGE PPAAPSGVGS LTMASGGGAP VADNNEGADG VGSSSGNWHC DSQWLGDRVI |
| | 135) | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSX₁QVX₂X₃X₄X₅ GQNQQTLKF |
| | | SVAGPSNMAV QGRNYIPGPS YRQQRVSTTV TQNNNSEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRYST RNL (SEQ ID NO: 263) |
| | | wherein X₁ is V or A, X₂ is S, G, V or A, X₃ is R or H, X₄ is any |
| | | amino acid and X₅ is S, G, V or A |
| AAV9.VR4- | X₁-Q-V- | MAADGYLPDW LEDNLSEGIR EWWALKPGAP QPKANQQHQD NARGLVLPGY KYLGPGNGLD |
| peptide | X₂-X₃-X₄- | KGEPVNAADA AALEHDKAYD QQLKAGDNPY LKYNHADAEF QERLKEDTSF GGNLGRAVFQ |
| 136 | X₅, (SEQ | AKKRLLEPLG LVEEAAKTAP GKKRPVEQSP QEPDSSAGIG KSGAQPAKKR LNFGQTGDTE |
| | ID NO: | SVPDPQPIGE PPAAPSGVGS LTMASGGGAP VADNNEGADG VGSSSGNWHC DSQWLGDRVI |
| | 136) | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSX₁QVX₂X₃X₄X₅ GQNQQTLKF |
| | | SVAGPSNMAV QGRNYIPGPS YRQQRVSTTV TQNNNSEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRYST RNL (SEQ ID NO: 264) |
| | | wherein X₁ is V or A, X₂ is S, G, V or A, X₃ is R or H, X₄ is any |
| | | amino acid and Xs is S or A |
| AAV9.VR4- | X₁-Q-V- | MAADGYLPDW LEDNLSEGIR EWWALKPGAP QPKANQQHQD NARGLVLPGY KYLGPGNGLD |
| peptide | X₂-X₃-X₄- | KGEPVNAADA AALEHDKAYD QQLKAGDNPY LKYNHADAEF QERLKEDTSF GGNLGRAVFQ |
| 137 | X₅, (SEQ | AKKRLLEPLG LVEEAAKTAP GKKRPVEQSP QEPDSSAGIG KSGAQPAKKR LNFGQTGDTE |
| | ID NO: | SVPDPQPIGE PPAAPSCVGS LTMASGGGAP VADNNEGADC VCSSSCNWHC DSQWLGDRVI |
| | 137) | TTSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSX₁QVX₂X₃X₄X₅ GQNQQTLKF |
| | | SVAGPSNMAV QGRNYIPGPS YRQQRVSTTV TQNNNSEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK RWNPEIQYTS |
| | | NYYKSNNVEF AVNTEGVYSE PRPIGTRYST RNL (SEQ ID NO: 265) |
| | | wherein X₁ is V or A, X₂ is S, G, V or A, X₃ is R or H, X₄ is T, S, |
| | | V, Y, A or P and X₅ is S, G, V or A |
| AAV9.VR4- | X₁-Q-V- | MAADGYLPDW LEDNLSEGIR EWWALKPGAP QPKANQQHQD NARGLVLPGY KYLGPGNGLD |
| peptide | X₂-X₃-X₄- | KGEPVNAADA AALEHDKAYD QQLKAGDNPY LKYNHADAEF QERLKEDTSF GGNLGRAVFQ |
| 138 | X₅, (SEQ | AKKRLLEPLG LVEEAAKTAP GKKRPVEQSP QEPDSSAGIG KSGAQPAKKR LNFGQTGDTE |
| | ID NO: | SVPDPQPIGE PPAAPSGVGS LTMASGGGAP VADNNEGADG VGSSSGNWHC DSQWLGDRVI |
| | 138) | ITSTRTWALP TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TLNDGSQAVG RSSFYCLEYF PSQMLRTGNN FQFSYEFENV |
| | | PFHSSYAHSQ SLDRLMNPLI DQYLYYLSKT INGSX₁QVX₂X₃X₄X₅ GQNQQTLKF |
| | | SVAGPSNMAV QGRNYIPGPS YRQQRVSTTV TQNNNSEFAW PGASSWALNG RNSLMNPGPA |
| | | MASHKEGEDR FFPLSGSLIF GKQGTGRDNV DADKVMITNE EEIKTTNPVA TESYGQVATN |
| | | HQSAQAQAQT GWVQNQGILP GMVWQDRDVY LQGPIWAKIP HTDGNFHPSP LMGGFGMKHP |
| | | PPQILIKNTP VPADPPTAFN KDKLNSFITQ YSTCQVSVEI EWELQKENSK RWNPEIQYTS |
| | | NYYKSNNVEF AVNTEGVYSE PRPIGTRYST RNL (SEQ ID NO: 266) |
| | | wherein X₁ is V or A, X₂ is S, G, V or A, X₃ is R or H, X₄ is T, S, |
| | | V, Y, A or P and X₅ is S or A |
| AAVhu32. | 497NNN499 | MAADGYLPDW LEDTLSEGIR QWWKLKPGPP PPKPAERHKD DSRGLVLPGY KYLGPGNGLD |
| AAA | substitution | KGEPVNAADA AALEHDKAYD QQLKAGDNPY LKYNHADAEF QERLKEDTSF GGNLGRAVFQ |
| | to AAA | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLCSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGQNQQ TLKFSVAGPS NMAVQGRNYI |
| | | PGPSYRQQRV STTVTQAAAS EFAWPGASSW ALNGRNSLMN PGPAMASHKE GEDRFFPLSG |
| | | SLIFGKQGTG RDNVDADKVM ITNEEEIKTT NPVATESYGQ VATNHQSAQA QACTGWVQNQ |
| | | GILPGMVWQD RDVYLQGPIW AKIPHTDGNF HPSPLMGGFG MKHPPPQILI KNTPVPADPP |
| | | TAFNKDKLNS FITQYSTGQV SVEIEWELQK ENSKRWNPEI QYTSNYYKSN NVEFAVNTEG |
| | | VYSEPRPIGT RYLTRNL (SEQ ID NO: 267) |
| AAVhu32. | SGTVIRS | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 1 | NO: 1) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGER PKRLNEKLFN LQVKEVTDNN GVKTIANNLT STVQVETDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSGTVIRS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEC VYSEPRPICT RYLTRNL (SEQ ID NO: 268) |
| AAVhu32. | GRPAP | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 2 | NO: 2) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGRPAP GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 269) |
| AAVhu32. | KDQKIGP | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 3 | NO: 3) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYEDENRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSKDQKIGP GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 270) |
| AAVhu32. | TRQYVPG | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 4 | NO: 4) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSTRQYVPG GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVELEWELQK ENSKRWNPEL QYTSNYYKSN |
| | | NVEFAVNTEG VYSEPRPIGT RYLTRNL (SEQ ID NO: 271) |
| AAVhu32. | VQVGRA | MAADGYLPDW LEDTLSEGIR QWWKLKPGPP PPKPAERHKD DSRGLVLPGY KYLGPGNGLD |
| AAA.VR4- | A (SEQ ID | KGEPVNAADA AALEHDKAYD QQLKAGDNPY LKYNHADAEF QERLKEDTSF GGNLGRAVFQ |
| peptide 5 | NO: 5) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVQVGRAA GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 272) |
| AAVhu32. | FTQSRGG | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 6 | NO: 6) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRIMNPL IDQYLYYLSK TINGSFTQSRGG GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 273) |
| AAVhu32. | SRSDRPA | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 7 | NO: 7) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRSDRPA GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 274) |
| AAVhu32. | VQVGRTS | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 8 | NO: 8) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVQVGRTS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 275) |
| AAVhu32. | SSGSQKF | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide 9 | NO: 9) | AKKRLLEPLG LVEEAAKTAP GKKRPVEQSP QEPDSSAGIG KSGSQPAKKK LNFGQTGDTE |
| | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSSGSQKE GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 276) |
| AAVhu32. | SGTVLRS | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 10) | AKKRLLEPLC LVEEAAKTAP GKKRPVEQSP QEPDSSAGIC KSCSQPAKKK LNFCQTGDTE |
| 10 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSGTVLRS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 277) |
| AAVhu32. | SHTKSAT | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 11) | AKKRLLEPLG LVEEAAKTAP GKKRPVEQSP QEPDSSAGIG KSGSQPAKKK LNFGQTGDTE |
| 11 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSHTKSAT GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 278) |
| AAVhu32. | DQARVRI | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 12) | AKKRLLEPLG LVEEAAKTAP GKKRPVEQSP QEPDSSAGIG KSGSQPAKKK LNFGQTGDTE |
| 12 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYEDENRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSDQARVRI GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 279) |
| AAVhu32. | RGAVQK | MAADGYLPDW LEDTLSEGIR QWWKLKPGPP PPKPAERHKD DSRGLVLPGY KYLGPGNGLD |
| AAA.VR4- | V (SEQ ID | KGEPVNAADA AALEHDKAYD QQLKAGDNPY LKYNHADAEF QERLKEDTSF GGNLGRAVFQ |
| peptide | NO: 13) | AKKRLLEPLG LVEEAAKTAP GKKRPVEQSP QEPDSSAGIG KSGSQPAKKK LNFGQTGDTE |
| 13 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSRGAVQKV GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVELEWELQK ENSKRWNPEL QYTSNYYKSN |
| | | NVEFAVNTEG VYSEPRPIGT RYLTRNL (SEQ ID NO: 280) |
| AAVhu32. | VGRDSRS | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 14) | AKKRLLEPLG LVEEAAKTAP GKKRPVEQSP QEPDSSAGIG KSGSQPAKKK LNFGQTGDTE |
| 14 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVGRDSRS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 281) |
| AAVhu32. | SRSDRQF | MAADGYLPDW LEDTLSEGIR QWWKLKPGPP PPKPAERHKD DSRGLVLPGY KYLGPGNGLD |
| AAA.VR4- | (SEQ ID | |
| peptide | NO: 15) | KGEPVNAADA AALEHDKAYD QQLKAGDNPY LKYNHADAEF QERLKEDTSF GGNLGRAVFQ |
| 15 | and | AKKRLLEPLG LVEEAAKTAP GKKRPVEQSP QEPDSSAGIG KSGSQPAKKK LNFGQTGDTE |
| | 497NNN499 | SVPDPGQPIG EPPAAPSGVG SLTMASGGGA PVADNNEGAD GVGSSSGNWH CDSQWLGDRV |
| | substitution | ITTSTRTWAL PTYNNHLYKQ ISNSTSGGSS NDNAYFGYST PWGYFDFNRF HCHFSPRDWQ |
| | to AAA | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRSDRQF GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 282) |
| AAVhu32. | VTRSQIM | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 16) | AKKRLLEPLG LVEEAAKTAP GKKRPVEQSP QEPDSSAGIG KSGSQPAKKK LNFGQTGDTE |
| 16 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVTRSQIM GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 283) |
| AAVhu32. | QVGARH | MAADGYLPDW LEDTLSEGIR QWWKLKPGPP PPKPAERHKD DSRGLVLPGY KYLGPGNGLD |
| AAA.VR4- | P (SEQ ID | KGEPVNAADA AALEHDKAYD QQLKAGDNPY LKYNHADAEF QERLKEDTSF GGNLGRAVFQ |
| peptide | NO: 17) | AKKRLLEPLG LVEEAAKTAP GKKRPVEQSP QEPDSSAGIG KSGSQPAKKK LNFGQTGDTE |
| 17 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSQVGARHP GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 284) |
| AAVhu32. | GRAAASL | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 18) | AKKRLLEPLG LVEEAAKTAP GKKRPVEQSP QEPDSSAGIG KSGSQPAKKK LNFGQTGDTE |
| 18 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGRAAASL GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 285) |
| AAVhu32. | PSGVPIR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 19) | AKKRLLEPLC LVEEAAKTAP CKKRPVEQSP QEPDSSAGIG KSGSQPAKKK LNFCQTGDTE |
| 19 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPSGVPIR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 286) |
| AAVhu32. | MSYNQA | MAADGYLPDW LEDTLSEGIR QWWKLKPGPP PPKPAERHKD DSRGLVLPGY KYLGPGNGLD |
| AAA.VR4- | T (SEQ ID | KGEPVNAADA AALEHDKAYD QQLKAGDNPY LKYNHADAEF QERLKEDTSF GGNLGRAVFQ |
| peptide | NO: 20) | AKKRLLEPLG LVEEAAKTAP GKKRPVEQSP QEPDSSAGIG KSGSQPAKKK LNFGQTGDTE |
| 20 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSMSYNQAT GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSC SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 287) |
| AAVhu32. | ITGGVRV | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 21) | AKKRLLEPLG LVEEAAKTAP GKKRPVEQSP QEPDSSAGIG KSGSQPAKKK LNFGQTGDTE |
| 21 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYEDENRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSITGGVRV GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 288) |
| AAVhu32. | SRSDRQV | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 22) | AKKRLLEPLG LVEEAAKTAP GKKRPVEQSP QEPDSSAGIG KSGSQPAKKK LNFGQTGDTE |
| 22 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRSDRQV GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVELEWELQK ENSKRWNPEL QYTSNYYKSN |
| | | NVEFAVNTEG VYSEPRPIGT RYLTRNL (SEQ ID NO: 289) |
| AAVhu32. | TSGVPIR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 23) | AKKRLLEPLG LVEEAAKTAP GKKRPVEQSP QEPDSSAGIG KSGSQPAKKK LNFGQTGDTE |
| 23 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSTSGVPIR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 290) |
| AAVhu32. | AQVGRA | MAADGYLPDW LEDTLSEGIR QWWKLKPGPP PPKPAERHKD DSRGLVLPGY KYLGPGNGLD |
| AAA.VR4- | S (SEQ ID | KGEPVNAADA AALEHDKAYD QQLKAGDNPY LKYNHADAEF QERLKEDTSF GGNLGRAVFQ |
| peptide | NO: 24) | AKKRLLEPLG LVEEAAKTAP GKKRPVEQSP QEPDSSAGIG KSGSQPAKKK LNFGQTGDTE |
| 24 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSAQVGRAS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 291) |
| AAVhu32. | VKASHHS | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 25) | AKKRLLEPLG LVEEAAKTAP GKKRPVEQSP QEPDSSAGIG KSGSQPAKKK LNFGQTGDTE |
| 25 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVKASHHS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 292) |
| AAVhu32. | SHTKTVT | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 26) | AKKRLLEPLG LVEEAAKTAP GKKRPVEQSP QEPDSSAGIG KSGSQPAKKK LNFGQTGDTE |
| 26 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSHTKTVT GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 293) |
| AAVhu32. | GSVQTVR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 27) | AKKRLLEPLG LVEEAAKTAP GKKRPVEQSP QEPDSSAGIG KSGSQPAKKK LNFGQTGDTE |
| 27 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGSVQTVR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 294) |
| AAVhu32. | DTRIRSM | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 28) | AKKRLLEPLC LVEEAAKTAP CKKRPVEQSP QEPDSSAGIG KSCSQPAKKK LNFCQTGDTE |
| 28 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSDTRIRSM GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 295) |
| AAVhu32. | SRTKTAT | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 29) | AKKRLLEPLG LVEEAAKTAP GKKRPVEQSP QEPDSSAGIG KSGSQPAKKK LNFGQTGDTE |
| 29 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRTKTAT GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSC SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 296) |
| AAVhu32. | QVGARQ | MAADGYLPDW LEDTLSEGIR QWWKLKPGPP PPKPAERHKD DSRGLVLPGY KYLGPGNGLD |
| AAA.VR4- | S (SEQ ID | KGEPVNAADA AALEHDKAYD QQLKAGDNPY LKYNHADAEF QERLKEDTSF GGNLGRAVFQ |
| peptide | NO: 30) | AKKRLLEPLG LVEEAAKTAP GKKRPVEQSP QEPDSSAGIG KSGSQPAKKK LNFGQTGDTE |
| 30 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYEDENRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSQVGARQS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 297) |
| AAVhu32. | SVTSVRV | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 37) | AKKRLLEPLG LVEEAAKTAP GKKRPVEQSP QEPDSSAGIG KSGSQPAKKK LNFGQTGDTE |
| 37 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSVTSVRV GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVETEWELQK ENSKRWNPEL QYTSNYYKSN |
| | | NVEFAVNTEG VYSEPRPIGT RYLTRNL (SEQ ID NO: 298) |
| AAVhu32. | MKYRGT | MAADGYLPDW LEDTLSEGIR QWWKLKPGPP PPKPAERHKD DSRGLVLPGY KYLGPGNGLD |
| AAA.VR4- | D (SEQ ID | KGEPVNAADA AALEHDKAYD QQLKAGDNPY LKYNHADAEF QERLKEDTSF GGNLGRAVFQ |
| peptide | NO: 45) | AKKRLLEPLG LVEEAAKTAP GKKRPVEQSP QEPDSSAGIG KSGSQPAKKK LNFGQTGDTE |
| 45 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSMKYRGTD GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 299) |
| AAVhu32. | SIAKNSA | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 76) | AKKRLLEPLG LVEEAAKTAP GKKRPVEQSP QEPDSSAGIG KSGSQPAKKK LNFGQTGDTE |
| 76 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSIAKNSA GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 300) |
| AAVhu32. | HMGQYG | MAADGYLPDW LEDTLSEGIR QWWKLKPGPP PPKPAERHKD DSRGLVLPGY KYLGPGNGLD |
| AAA.VR4- | K (SEQ ID | KGEPVNAADA AALEHDKAYD QQLKAGDNPY LKYNHADAEF QERLKEDTSF GGNLGRAVFQ |
| peptide | NO:82) | AKKRLLEPLG LVEEAAKTAP GKKRPVEQSP QEPDSSAGIG KSGSQPAKKK LNFGQTGDTE |
| 82 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSHMGQYGK GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 301) |
| AAVhu32. | MRAVSQ | MAADGYLPDW LEDTLSEGIR QWWKLKPGPP PPKPAERHKD DSRGLVLPGY KYLGPGNGLD |
| AAA.VR4- | L (SEQ ID | KGEPVNAADA AALEHDKAYD QQLKAGDNPY LKYNHADAEF QERLKEDTSF GGNLGRAVFQ |
| peptide | NO: 109) | AKKRLLEPLG LVEEAAKTAP GKKRPVEQSP QEPDSSAGIG KSGSQPAKKK LNFGQTGDTE |
| 109 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVJGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSMRAVSQL GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 302) |
| AAVhu32. | PRQYVPG | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 110) | AKKRLLEPLG LVEEAAKTAP GKKRPVEQSP QEPDSSAGIG KSGSQPAKKK LNFGQTGDTE |
| 110 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPRQYVPG GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 303) |
| AAVhu32. | RSSSGR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 111) | AKKRLLEPLC LVEEAAKTAP CKKRPVEQSP QEPDSSAGIG KSCSQPAKKK LNFGQTGDTE |
| 111 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSRSSSGR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 304) |
| AAVhu32. | VVKSTKS | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 112) | AKKRLLEPLG LVEEAAKTAP GKKRPVEQSP QEPDSSAGIG KSGSQPAKKK LNFGQTGDTE |
| 112 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVJGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVVKSTKS GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFCKQCTC RDNVDADKVM ITNEEEIKTT NPVATESYCQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 305) |
| AAVhu32. | PSSVQHR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 118) | AKKRLLEPLG LVEEAAKTAP GKKRPVEQSP QEPDSSAGIG KSGSQPAKKK LNFGQTGDTE |
| 118 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYEDENRY HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPSSVQHR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 306) |
| AAVhu32. | SSSVQHR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 119) | AKKRLLEPLG LVEEAAKTAP GKKRPVEQSP QEPDSSAGIG KSGSQPAKKK LNFGQTGDTE |
| 119 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSSSVQHR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVELEWELQK ENSKRWNPEL QYTSNYYKSN |
| | | NVEFAVNTEG VYSEPRPIGT RYLTRNL (SEQ ID NO: 307) |
| AAVhu32. | SGMQER | MAADGYLPDW LEDTLSEGIR QWWKLKPGPP PPKPAERHKD DSRGLVLPGY KYLGPGNGLD |
| AAA.VR4- | R (SEQ ID | KGEPVNAADA AALEHDKAYD QQLKAGDNPY LKYNHADAEF QERLKEDTSF GGNLGRAVFQ |
| peptide | NO: 120) | AKKRLLEPLG LVEEAAKTAP GKKRPVEQSP QEPDSSAGIG KSGSQPAKKK LNFGQTGDTE |
| 120 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSGMQERR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 308) |
| AAVhu32. | LERGNLE | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 121) | AKKRLLEPLG LVEEAAKTAP GKKRPVEQSP QEPDSSAGIG KSGSQPAKKK LNFGQTGDTE |
| 121 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNEGSQAV GRSSFYCLEY FPSQMLRTGN NFQFSYEFEN |
| | | VPFHSSYAHS QSLDRLMNPL IDQYLYYLSK TINGSLERGNLE GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 309) |
| AAVhu32. | YRDVRQ | MAADGYLPDW LEDTLSEGIR QWWKLKPGPP PPKPAERHKD DSRGLVLPGY KYLGPGNGLD |
| AAA.VR4- | T (SEQ ID | KGEPVNAADA AALEHDKAYD QQLKAGDNPY LKYNHADAEF QERLKEDTSF GGNLGRAVFQ |
| peptide | NO: 122) | AKKRLLEPLG LVEEAAKTAP GKKRPVEQSP QEPDSSAGIG KSGSQPAKKK LNFGQTGDTE |
| 122 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSYRDVRQT GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 310) |
| AAVhu32. | PSAVQHR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 123) | AKKRLLEPLG LVEEAAKTAP GKKRPVEQSP QEPDSSAGIG KSGSQPAKKK LNFGQTGDTE |
| 123 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPSAVQHR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 311) |
| AAVhu32. | YVGGRA | MAADGYLPDW LEDTLSEGIR QWWKLKPGPP PPKPAERHKD DSRGLVLPGY KYLGPGNGLD |
| AAA.VR4- | V (SEQ ID | KGEPVNAADA AALEHDKAYD QQLKAGDNPY LKYNHADAEF QERLKEDTSF GGNLGRAVFQ |
| peptide | NO: 124) | AKKRLLEPLG LVEEAAKTAP GKKRPVEQSP QEPDSSAGIG KSGSQPAKKK LNFGQTGDTE |
| 124 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSYVGGRAV GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 312) |
| AAVhu32. | IGSRGVA | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 125) | AKKRLLEPLC LVEEAAKTAP CKKRPVEQSP QEPDSSAGIG KSCSQPAKKK LNFCQTGDTE |
| 125 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSIGSRGVA GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 313) |
| AAVhu32. | SDVSRPR | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 126) | AKKRLLEPLG LVEEAAKTAP GKKRPVEQSP QEPDSSAGIG KSGSQPAKKK LNFGQTGDTE |
| 126 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSDVSRPR GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSC SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 314) |
| AAVhu32. | GSVRQA | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 127) | AKKRLLEPLG LVEEAAKTAP GKKRPVEQSP QEPDSSAGIG KSGSQPAKKK LNFGQTGDTE |
| 127 | and | SVPDPGQPIG EPPAAPSGVG SLTMASGGGA PVADNNEGAD GVGSSSGNWH CDSQWLGDRV |
| | 497NNN499 | ITTSTRIWAL PTYNNHLYKQ ISNSTSGGSS NDNAYFGYST PWGYEDENRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGSVRQAA GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 315) |
| AAVhu32. | QSPHTSQ | MAADGYLPDW LEDTLSEGIR QWWKLKPGPP PPKPAERHKD DSRGLVLPGY KYLGPGNGLD |
| AAA.VR4- | (SEQ ID | KGEPVNAADA AALEHDKAYD QQLKAGDNPY LKYNHADAEF QERLKEDTSF GGNLGRAVFQ |
| peptide | NO: 128) | AKKRLLEPLG LVEEAAKTAP GKKRPVEQSP QEPDSSAGIG KSGSQPAKKK LNFGQTGDTE |
| 128 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSQSPHTSQ GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVETEWELQK ENSKRWNPEL QYTSNYYKSN |
| | | NVEFAVNTEG VYSEPRPIGT RYLTRNL (SEQ ID NO: 316) |
| AAVhu32. | ASQAYH | MAADGYLPDW LEDTLSEGIR QWWKLKPGPP PPKPAERHKD DSRGLVLPGY KYLGPGNGLD |
| AAA.VR4- | G (SEQ ID | KGEPVNAADA AALEHDKAYD QQLKAGDNPY LKYNHADAEF QERLKEDTSF GGNLGRAVFQ |
| peptide | NO: 129) | AKKRLLEPLG LVEEAAKTAP GKKRPVEQSP QEPDSSAGIG KSGSQPAKKK LNFGQTGDTE |
| 129 | and | SVPDPGQPIG EPPAAPSGVG SLTMASGGGA PVADNNEGAD GVGSSSGNWH CDSQWLGDRV |
| | 497NNN499 | ITTSTRTWAL PTYNNHLYKQ ISNSTSGGSS NDNAYFGYST PWGYFDFNRF HCHFSPRDWQ |
| | substitution | RLINNNWGFR PKRLNFKLFN IQVKEVTDNN GVKTIANNLT STVQVFTDSD YQLPYVLGSA |
| | to AAA | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSASQAYHG GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 317) |
| AAVhu32. | X₁-Q-V- | MAADGYLPDW LEDTLSEGIR QWWKLKPGPP PPKPAERHKD DSRGLVLPGY KYLGPGNGLD |
| AAA.VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 135 | ID NO: | SVPDPGQPIG EPPAAPSGVG SLTMASGGGA PVADNNEGAD GVGSSSGNWH CDSQWLGDRV |
| | 135) | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRIMNPL IDQYLYYLSK TINGSX₁QVX₂X₃X₄X₅ GQNQQ TLKFSVAGPS |
| | | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 318) |
| | | wherein X₁ is V or A, X₂ is G or V or A, X₃ is R or H, X₄ is any |
| | | amino acid and X₅ is S, G, V or A |
| AAVhu32. | X₁-Q-V- | MAADGYLPDW LEDTLSEGIR QWWKLKPGPP PPKPAERHKD DSRGLVLPGY KYLGPGNGLD |
| AAA.VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 136 | ID NO: | SVPDPGQPIG EPPAAPSGVG SLTMASGGGA PVADNNEGAD GVGSSSGNWH CDSQWLGDRV |
| | 136) | ITTSTRTWAL PTYNNHLYKQ ISNSTSGGSS NDNAYFGYST PWGYEDENRF HCHFSPRDWQ |
| | and | RLINNNWGFR PKRLNFKLFN IQVKEVTDNN GVKTIANNLT STVQVFTDSD YQLPYVLGSA |
| | 497NNN499 | HEGCLPPFPA DVFMIPQYGY LTLNDGSQAV GRSSFYCLEY FPSQMLRTGN NFQFSYEFEN |
| | substitution | VPFHSSYAHS QSLDRLMNPL IDQYLYYLSK TINGSX₁QVX₂X₃X₄X₅ GQNQQ TLKFSVAGPS |
| | to AAA | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 319) |
| | | wherein X₁ is V or A, X₂ is S, G, V or A, X₃ is R or H, X₄ is any |
| | | amino acid and X₅ is S or A |
| AAVhu32. | X₁-Q-V- | MAADGYLPDW LEDTLSEGIR QWWKLKPGPP PPKPAERHKD DSRGLVLPGY KYLGPGNGLD |
| AAA.VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 137 | ID NO: | SVPDPGQPIG EPPAAPSGVG SLTMASGGGA PVADNNEGAD GVGSSSGNWH CDSQWLGDRV |
| | 137) | ITTSTRTWAL PTYNNHLYKQ ISNSTSGGSS NDNAYFGYST PWGYFDFNRF HCHFSPRDWQ |
| | and | RLINNNWGFR PKRLNFKLFN IQVKEVTDNN GVKTIANNLT STVQVFTDSD YQLPYVLGSA |
| | 497NNN499 | HEGCLPPFPA DVFMIPQYGY LTLNDGSQAV GRSSFYCLEY FPSQMLRTGN NFQFSYEFEN |
| | substitution | VPFHSSYAHS QSLDRLMNPL IDQYLYYLSK TINGSX₁QVX₂X₃X₄X₅ GQNQQ TLKFSVAGPS |
| | to AAA | NMAVQGRNYI PGPSYRQQRV STTVTQAAAS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 320) |
| | | wherein X₁ is V or A, X₂ is S, G, V or A, X₃ is R or H, X₄ is T, S, |
| | | V, Y, A or P and X₅ is S, G, V or A |
| AAVhu32. | X₁-Q-V- | MAADGYLPDW LEDTLSEGIR QWWKLKPGPP PPKPAERHKD DSRGLVLPGY KYLGPGNGLD |
| AAA.VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 138 | ID NO: | SVPDPGQPIG EPPAAPSGVG SLTMASGGGA PVADNNEGAD GVGSSSGNWH CDSQWLGDRV |
| | 138) | ITTSTRTWAL PTYNNHLYKQ ISNSTSGGSS NDNAYFGYST PWGYFDFNRF HCHFSPRDWQ |
| | and | RLINNNWGFR PKRLNFKLFN IQVKEVTDNN GVKTIANNLT STVQVFTDSD YQLPYVLGSA |
| | 497NNN499 | HEGCLPPFPA DVFMIPQYGY LTLNDGSQAV GRSSFYCLEY FPSQMLRTGN NFQFSYEFEN |
| | substitution | VPFHSSYAHS QSLDRLMNPL IDQYLYYLSK TINGSX₁QVX₂X₃X₄X₅ GQNQQ TLKFSVAGPS |
| | to AAA | NMAVQGRNYI PGPSYRQQRV STTVTQAAAS EFAWPGASSW ALNGRNSLMN PGPAMASHKE |
| | | GEDRFFPLSC SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 321) |
| | | wherein X₁ is V or A, X₂ is S, G, V or A, X₃ is R or H, X₄ is T, S, |
| | | V, Y, A or P and X₅ is S or A |
| AAVhu32. | SGTVIRS | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 1 | NO: 1) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSGTVIRS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 322) |
| AAVhu32. | GRPAP | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 2 | NO: 2) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGRPAP GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTCQV SVEIEWELQK ENSKRWNPEI QYTSNYYKSN |
| | | NVEFAVNTEG VYSEPRPIGT RYLTRNL (SEQ ID NO: 323) |
| AAVhu32. | KDQKIGP | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 3 | NO: 3) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSKDQKIGP GQNQQ TLKFSVAGPS |
| | | NMAVQGRNYI PCPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVELEWELQK ENSKRWNPEL QYTSNYYKSN |
| | | NVEFAVNTEG VYSEPRPIGT RYLTRNL (SEQ ID NO: 324) |
| AAVhu32. | TRQYVPG | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 4 | NO: 4) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSTRQYVPG GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 325) |
| AAVhu32. | VQVGRA | MAADGYLPDW LEDTLSEGIR QWWKLKPGPP PPKPAERHKD DSRGLVLPGY KYLGPGNGLD |
| VR4- | A (SEQ ID | KGEPVNAADA AALEHDKAYD QQLKAGDNPY LKYNHADAEF QERLKEDTSF GGNLGRAVFQ |
| peptide 5 | NO: 5) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVQVGRAA GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 326) |
| AAVhu32. | FTQSRGG | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 6 | NO: 6) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSFTQSRGG GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 327) |
| AAVhu32. | SRSDRPA | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 7 | NO: 7) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRSDRPA GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 328) |
| AAVhu32. | VQVGRTS | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 8 | NO: 8) | AKKRLLEPLG LVEEAAKTAP GKKRPVEQSP QEPDSSAGIG KSGSQPAKKK LNFGQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVQVGRTS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 329) |
| AAVhu32. | SSGSQKF | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide 9 | NO: 9) | AKKRLLEPLC LVEEAAKTAP CKKRPVEQSP QEPDSSAGIG KSGSQPAKKK LNFCQTGDTE |
| | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSSGSQKF GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 330) |
| AAVhu32. | SGTVLRS | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 10) | AKKRLLEPLG LVEEAAKTAP GKKRPVEQSP QEPDSSAGIG KSGSQPAKKK LNFGQTGDTE |
| 10 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSGTVLRS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 331) |
| AAVhu32. | SHTKSAT | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 11) | AKKRLLEPLG LVEEAAKTAP GKKRPVEQSP QEPDSSAGIG KSGSQPAKKK LNFGQTGDTE |
| 11 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDCSQAV GRSSFYCLEY FPSQMLRTGN NFQFSYEFEN |
| | | VPFHSSYAHS QSLDRLMNPL IDQYLYYLSK TINGSSHTKSAT GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 332) |
| AAVhu32. | DQARVRI | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 12) | AKKRLLEPLG LVEEAAKTAP GKKRPVEQSP QEPDSSAGIG KSGSQPAKKK LNFGQTGDTE |
| 12 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSDQARVRI GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 333) |
| AAVhu32. | RGAVQK | MAADGYLPDW LEDTLSEGIR QWWKLKPGPP PPKPAERHKD DSRGLVLPGY KYLGPGNGLD |
| VR4- | V (SEQ ID | KGEPVNAADA AALEHDKAYD QQLKAGDNPY LKYNHADAEF QERLKEDTSF GGNLGRAVFQ |
| peptide | NO: 13) | AKKRLLEPLG LVEEAAKTAP GKKRPVEQSP QEPDSSAGIG KSGSQPAKKK LNFGQTGDTE |
| 13 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSRGAVQKV GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 334) |
| AAVhu32. | VGRDSRS | MAADGYLPDW LEDTLSEGIR QWWKLKPGPP PPKPAERHKD DSRGLVLPGY KYLGPGNGLD |
| VR4- | (SEQ ID | KCEPVNAADA AALEHDKAYD QQLKAGDNPY LKYNHADAEF QERLKEDTSF GGNLGRAVFQ |
| peptide | NO: 14) | AKKRLLEPLG LVEEAAKTAP GKKRPVEQSP QEPDSSAGIG KSGSQPAKKK LNFGQTGDTE |
| 14 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVGRDSRS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 335) |
| AAVhu32. | SRSDRQF | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAET QERLKEDTSE GGNLGRAVFQ |
| peptide | NO: 15) | AKKRLLEPLG LVEEAAKTAP GKKRPVEQSP QEPDSSAGIG KSGSQPAKKK LNFGQTGDTE |
| 15 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRSDRQF GQNQQ TLKFSVAGPS |
| | | NMAVQGRNYI PCPSYRQQRV STTVTQNNNS EFAWPCASSW ALNGRNSLMN PCPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 336) |
| AAVhu32. | VTRSQIM | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 16) | AKKRLLEPLG LVEEAAKTAP GKKRPVEQSP QEPDSSAGIG KSGSQPAKKK LNFGQTGDTE |
| 16 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVTRSQIM GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 337) |
| AAVhu32. | QVGARH | MAADGYLPDW LEDTLSEGIR QWWKLKPGPP PPKPAERHKD DSRGLVLPGY KYLGPGNGLD |
| VR4- | P (SEQ ID | KGEPVNAADA AALEHDKAYD QQLKAGDNPY LKYNHADAEF QERLKEDTSF GGNLGRAVFQ |
| peptide | NO: 17) | AKKRLLEPLG LVEEAAKTAP GKKRPVEQSP QEPDSSAGIG KSGSQPAKKK LNFGQTGDTE |
| 17 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSQVGARHP CQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 338) |
| AAVhu32. | GRAAASL | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 18) | AKKRLLEPLG LVEEAAKTAP GKKRPVEQSP QEPDSSAGIG KSGSQPAKKK LNFGQTGDTE |
| 18 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGRAAASL GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 339) |
| AAVhu32. | PSGVPIR | MAADGYLPDW LEDTLSEGIR QWWKLKPCPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 19) | AKKRLLEPLG LVEEAAKTAP GKKRPVEQSP QEPDSSAGIG KSGSQPAKKK LNFGQTGDTE |
| 19 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFCESYEFEN |
| | | VPFHSSYAHS QSLDRLMNPL IDQYLYYLSK TINGSPSGVPIR GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 340) |
| AAVhu32. | MSYNQA | MAADGYLPDW LEDTLSEGIR QWWKLKPGPP PPKPAERHKD DSRGLVLPGY KYLGPGNGLD |
| VR4- | T (SEQ ID | KGEPVNAADA AALEHDKAYD QQLKAGDNPY LKYNHADAEF QERLKEDTSF GGNLGRAVFQ |
| peptide | NO: 20) | AKKRLLEPLG LVEEAAKTAP GKKRPVEQSP QEPDSSAGIG KSGSQPAKKK LNFGQTGDTE |
| 20 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSMSYNQAT GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 341) |
| AAVhu32. | ITGGVRV | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 21) | AKKRLLEPLG LVEEAAKTAP GKKRPVEQSP QEPDSSAGIG KSGSQPAKKK LNFGQTGDTE |
| 21 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSITGGVRV GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 342) |
| AAVhu32. | SRSDRQV | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 22) | AKKRLLEPLG LVEEAAKTAP GKKRPVEQSP QEPDSSAGIG KSGSQPAKKK LNFGQTGDTE |
| 22 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRSDRQV GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 343) |
| AAVhu32. | TSGVPIR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 23) | AKKRLLEPLG LVEEAAKTAP GKKRPVEQSP QEPDSSAGIG KSGSQPAKKK LNFGQTGDTE |
| 23 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSTSGVPIR GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 344) |
| AAVhu32. | AQVGRA | MAADGYLPDW LEDTLSEGIR QWWKLKPGPP PPKPAERHKD DSRGLVLPGY KYLGPGNGLD |
| VR4- | S (SEQ ID | KGEPVNAADA AALEHDKAYD QQLKAGDNPY LKYNHADAEF QERLKEDTSF GGNLGRAVFQ |
| peptide | NO: 24) | AKKRLLEPLG LVEEAAKTAP GKKRPVEQSP QEPDSSAGIG KSGSQPAKKK LNFGQTGDTE |
| 24 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSAQVGRAS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 345) |
| AAVhu32. | VKASHHS | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 25) | AKKRLLEPLG LVEEAAKTAP GKKRPVEQSP QEPDSSAGIG KSGSQPAKKK LNFGQTGDTE |
| 25 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVKASHHS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVBADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 346) |
| AAVhu32. | SHTKTVT | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 26) | AKKRLLEPLG LVEEAAKTAP GKKRPVEQSP QEPDSSAGIG KSGSQPAKKK LNFGQTGDTE |
| 26 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVEMIPQYGY LTLNDGSQAV GRSSFYCLEY FPSQMLRIGN NEQFSYEFEN |
| | | VPFHSSYAHS QSLDRLMNPL IDQYLYYLSK TINGSSHTKTVT GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAENKDKLNS FITQYSTGQV SVELEWELQK ENSKRWNPEL QYTSNYYKSN |
| | | NVEFAVNTEG VYSEPRPIGT RYLTRNL (SEQ ID NO: 347) |
| AAVhu32. | GSVQTVR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 27) | AKKRLLEPLG LVEEAAKTAP GKKRPVEQSP QEPDSSAGIG KSGSQPAKKK LNFGQTGDTE |
| 27 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGSVQTVR GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 348) |
| AAVhu32. | DTRIRSM | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 28) | AKKRLLEPLG LVEEAAKTAP GKKRPVEQSP QEPDSSAGIG KSGSQPAKKK LNFGQTGDTE |
| 28 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSDTRIRSM GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 349) |
| AAVhu32. | SRTKTAT | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAET QERLKEDTSE GGNLGRAVFQ |
| peptide | NO: 29) | AKKRLLEPLG LVEEAAKTAP GKKRPVEQSP QEPDSSAGIG KSGSQPAKKK LNFGQTGDTE |
| 29 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSRTKTAT GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 350) |
| AAVhu32. | QVGARQ | MAADGYLPDW LEDTLSEGIR QWWKLKPGPP PPKPAERHKD DSRGLVLPGY KYLGPGNGLD |
| VR4- | S (SEQ ID | KGEPVNAADA AALEHDKAYD QQLKAGDNPY LKYNHADAEF QERLKEDTSF GGNLGRAVFQ |
| peptide | NO: 30) | AKKRLLEPLG LVEEAAKTAP GKKRPVEQSP QEPDSSAGIG KSGSQPAKKK LNFGQTGDTE |
| 30 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLCSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSQVGARQS GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 351) |
| AAVhu32. | SVTSVRV | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 37) | AKKRLLEPLG LVEEAAKTAP GKKRPVEQSP QEPDSSAGIG KSGSQPAKKK LNFGQTGDTE |
| 37 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGER PKRLNFKLFN LQVKEVIDNN GVKTIANNLT STVQVETDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSVTSVRV CQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 352) |
| AAVhu32. | MKYRGT | MAADGYLPDW LEDTLSEGIR QWWKLKPGPP PPKPAERHKD DSRGLVLPGY KYLGPGNGLD |
| VR4- | D (SEQ ID | KGEPVNAADA AALEHDKAYD QQLKAGDNPY LKYNHADAEF QERLKEDTSF GGNLGRAVFQ |
| peptide | NO: 45) | AKKRLLEPLG LVEEAAKTAP GKKRPVEQSP QEPDSSAGIG KSGSQPAKKK LNFGQTGDTE |
| 45 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSMKYRGTD GQNQQ TLKESVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 353) |
| AAVhu32. | SIAKNSA | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 76) | AKKRLLEPLG LVEEAAKTAP GKKRPVEQSP QEPDSSAGIG KSGSQPAKKK LNFGQTGDTE |
| 76 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSIAKNSA GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 354) |
| AAVhu32. | HMGQYG | MAADGYLPDW LEDTLSEGIR QWWKLKPGPP PPKPAERHKD DSRGLVLPGY KYLGPGNGLD |
| VR4- | K (SEQ ID | KGEPVNAADA AALEHDKAYD QQLKAGDNPY LKYNHADAEF QERLKEDTSF GGNLGRAVFQ |
| peptide | NO:82) | AKKRLLEPLG LVEEAAKTAP GKKRPVEQSP QEPDSSAGIG KSGSQPAKKK LNFGQTGDTE |
| 82 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSHMGQYGK GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 355) |
| AAVhu32. | MRAVSQ | MAADGYLPDW LEDTLSEGIR QWWKLKPGPP PPKPAERHKD DSRGLVLPGY KYLGPGNGLD |
| VR4- | L (SEQ ID | KGEPVNAADA AALEHDKAYD QQLKAGDNPY LKYNHADAEF QERLKEDTSF GGNLGRAVFQ |
| peptide | NO: 109) | AKKRLLEPLG LVEEAAKTAP GKKRPVEQSP QEPDSSAGIG KSGSQPAKKK LNFGQTGDTE |
| 109 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWCYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSMRAVSQL GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 356) |
| AAVhu32. | PRQYVPG | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 110) | AKKRLLEPLG LVEEAAKTAP GKKRPVEQSP QEPDSSAGIG KSGSQPAKKK LNFGQTGDTE |
| 110 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPRQYVPG GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 357) |
| AAVhu32. | RSSSGR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 111) | AKKRLLEPLG LVEEAAKTAP GKKRPVEQSP QEPDSSAGIG KSGSQPAKKK LNFGQTGDTE |
| 111 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSRSSSGR GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 358) |
| AAVhu32. | VVKSTKS | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 112) | AKKRLLEPLG LVEEAAKTAP GKKRPVEQSP QEPDSSAGIG KSGSQPAKKK LNFGQTGDTE |
| 112 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSVVKSTKS GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 359) |
| AAVhu32. | PSSVQHR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 118) | AKKRLLEPLG LVEEAAKTAP GKKRPVEQSP QEPDSSAGIG KSGSQPAKKK LNFGQTGDTE |
| 118 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPSSVQHR GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 360) |
| AAVhu32. | SSSVQHR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 119) | AKKRLLEPLG LVEEAAKTAP GKKRPVEQSP QEPDSSAGIG KSGSQPAKKK LNFGQTGDTE |
| 119 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSSSVQHR GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 361) |
| AAVhu32. | SGMQER | MAADGYLPDW LEDTLSEGIR QWWKLKPGPP PPKPAERHKD DSRGLVLPGY KYLGPGNGLD |
| VR4- | R (SEQ ID | KGEPVNAADA AALEHDKAYD QQLKAGDNPY LKYNHADAEF QERLKEDTSF GGNLGRAVFQ |
| peptide | NO: 120) | AKKRLLEPLG LVEEAAKTAP GKKRPVEQSP QEPDSSAGIG KSGSQPAKKK LNFGQTGDTE |
| 120 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSGMQERR GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 362) |
| AAVhu32. | LERGNLE | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 121) | AKKRLLEPLG LVEEAAKTAP GKKRPVEQSP QEPDSSAGIG KSGSQPAKKK LNFGQTGDTE |
| 121 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSLERGNLE GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 363) |
| AAVhu32. | YRDVRQ | MAADGYLPDW LEDTLSEGIR QWWKLKPGPP PPKPAERHKD DSRGLVLPGY KYLGPGNGLD |
| VR4- | T (SEQ ID | KGEPVNAADA AALEHDKAYD QQLKAGDNPY LKYNHADAEF QERLKEDTSF GGNLGRAVFQ |
| peptide | NO: 122) | AKKRLLEPLG LVEEAAKTAP GKKRPVEQSP QEPDSSAGIG KSGSQPAKKK LNFGQTGDTE |
| 122 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSYRDVRQT GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 364) |
| AAVhu32. | PSAVQHR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 123) | AKKRLLEPLG LVEEAAKTAP GKKRPVEQSP QEPDSSAGIG KSGSQPAKKK LNFGQTGDTE |
| 123 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSPSAVQHR GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 365) |
| AAVhu32. | YVGGRA | MAADGYLPDW LEDTLSEGIR QWWKLKPGPP PPKPAERHKD DSRGLVLPGY KYLGPGNGLD |
| VR4- | V (SEQ ID | KGEPVNAADA AALEHDKAYD QQLKAGDNPY LKYNHADAEF QERLKEDTSF GGNLGRAVFQ |
| peptide | NO: 124) | AKKRLLEPLG LVEEAAKTAP GKKRPVEQSP QEPDSSAGIG KSGSQPAKKK LNFGQTGDTE |
| 124 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYEDENRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSYVGGRAV GQNQQ TLKFSVAGPS |
| | | NMAVQGRNYI PGPSYRQQRV STTVTQNNNS EFAWPGASSW ALNGRNSLMN PGPAMASHKE |
| | | GEDRFEPLSG SLIFGKQGTG RDNVLADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 366) |
| AAVhu32. | IGSRGVA | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 125) | AKKRLLEPLG LVEEAAKTAP GKKRPVEQSP QEPDSSAGIG KSGSQPAKKK LNFGQTGDTE |
| 125 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSIGSRGVA GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 367) |
| AAVhu32. | SDVSRPR | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 126) | AKKRLLEPLG LVEEAAKTAP GKKRPVEQSP QEPDSSAGIG KSGSQPAKKK LNFGQTGDTE |
| 126 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSSDVSRPR GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 368) |
| AAVhu32. | GSVRQA | MAADGYLPDW LEDTLSEGIR QWWKLKPGPP PPKPAERHKD DSRGLVLPGY KYLGPGNGLD |
| VR4- | A (SEQ ID | KGEPVNAADA AALEHDKAYD QQLKAGDNPY LKYNHADAEF QERLKEDTSF GGNLGRAVFQ |
| peptide | NO: 127) | AKKRLLEPLC LVEEAAKTAP CKKRPVEQSP QEPDSSAGIG KSGSQPAKKK LNFCQTGDTE |
| 127 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSGSVRQAA GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 369) |
| AAVhu32. | QSPHTSQ | MAADGYLPDW LEDTLSEGIR QWWKLKPGPP PPKPAERHKD DSRGLVLPGY KYLGPGNGLD |
| VR4- | (SEQ ID | KGEPVNAADA AALEHDKAYD QQLKAGDNPY LKYNHADAEF QERLKEDTSF GGNLGRAVFQ |
| peptide | NO: 128) | AKKRLLEPLG LVEEAAKTAP GKKRPVEQSP QEPDSSAGIG KSGSQPAKKK LNFGQTGDTE |
| 128 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSQSPHTSQ GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 370) |
| AAVhu32. | ASQAYH | MAADGYLPDW LEDTLSEGIR QWWKLKPGPP PPKPAERHKD DSRGLVLPGY KYLGPGNGLD |
| VR4- | G (SEQ ID | KGEPVNAADA AALEHDKAYD QQLKAGDNPY LKYNHADAEF QERLKEDTSF GGNLGRAVFQ |
| peptide | NO: 129) | AKKRLLEPLG LVEEAAKTAP GKKRPVEQSP QEPDSSAGIG KSGSQPAKKK LNFGQTGDTE |
| 129 | | SVPDPGQPIG EPPAAPSGVG SLTMASGGGA PVADNNEGAD GVGSSSGNWH CDSQWLGDRV |
| | | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSASQAYHG GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 371) |
| AAVhu32. | X₁-Q-V- | MAADGYLPDW LEDTLSEGIR QWWKLKPGPP PPKPAERHKD DSRGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 135 | ID NO: | SVPDPGQPIG EPPAAPSGVG SLTMASGGGA PVADNNEGAD GVGSSSGNWH CDSQWLGDRV |
| | 135) | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSX₁QVX₂X₃X₄X₅ GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 372) |
| | | wherein X₁ is V or A, X₂ is G or V or A, X₃ is R or H, X₄ is any |
| | | amino acid and Xs is S, G, V or A |
| AAVhu32. | X₁-Q-V- | MAADGYLPDW LEDTLSEGIR QWWKLKPGPP PPKPAERHKD DSRGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 136 | ID NO: | SVPDPGQPIG EPPAAPSGVG SLTMASGGGA PVADNNEGAD GVGSSSGNWH CDSQWLGDRV |
| | 136) | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSX₁QVX₂X₃X₄X₅ GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 373) |
| | | wherein X₁ is V or A, X₂ is S, G, V or A, X₃ is R or H, X₄ is any |
| | | amino acid and X₅ is S or A |
| AAVhu32. | X₁-Q-V- | MAADGYLPDW LEDTLSEGIR QWWKLKPGPP PPKPAERHKD DSRGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 137 | ID NO: | SVPDPGQPIG EPPAAPSGVG SLTMASGGGA PVADNNEGAD GVGSSSGNWH CDSQWLGDRV |
| | 137) | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSX₁QVX₂X₃X₄X₅ GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 374) |
| | | wherein X₁ is V or A, X₂ is S, G, V or A, X₃ is R or H, X₄ is T, S, |
| | | V, Y, A or P and X₅ is S, G, V or A |
| AAVhu32. | X₁-Q-V- | MAADGYLPDW LEDTLSEGIR QWWKLKPGPP PPKPAERHKD DSRGLVLPGY KYLGPGNGLD |
| VR4- | X₂-X₃-X₄- | KGEPVNAADA AALEHDKAYD QQLKAGDNPY LKYNHADAEF QERLKEDTSF GGNLGRAVFQ |
| peptide | X₅, (SEQ | AKKRLLEPLG LVEEAAKTAP GKKRPVEQSP QEPDSSAGIG KSGSQPAKKK LNFGQTGDTE |
| 138 | ID NO: | SVPDPGQPIG EPPAAPSGVG SLTMASGGGA PVADNNEGAD GVGSSSGNWH CDSQWLGDRV |
| | 138) | ITTSTRTWAL PTYNNHLYKQ ISNSTSGGSS NDNAYFGYST PWGYFDFNRF HCHFSPRDWQ |
| | | RLINNNWGFR PKRLNFKLFN IQVKEVTDNN GVKTIANNLT STVQVFTDSD YQLPYVLGSA |
| | | HEGCLPPFPA DVFMIPQYGY LTLNDGSQAV GRSSFYCLEY FPSQMLRTGN NFQFSYEFEN |
| | | VPFHSSYAHS QSLDRLMNPL IDQYLYYLSK TINGSX₁QVX₂X₃X₄X₅ GQNQQ TLKFSVAGPS |
| | | NMAVQGRNYI PGPSYRQQRV STTVTQNNNS EFAWPGASSW ALNGRNSLMN PGPAMASHKE |
| | | GEDRFFPLSG SLIFGKQGTG RDNVDADKVM ITNEEEIKTT NPVATESYGQ VATNHQSAQA |
| | | QAQTGWVQNQ GILPGMVWQD RDVYLQGPIW AKIPHTDGNF HPSPLMGGFG MKHPPPQILI |
| | | KNTPVPADPP TAFNKDKLNS FITQYSTGQV SVEIEWELQK ENSKRWNPEI QYTSNYYKSN |
| | | NVEFAVNTEG VYSEPRPIGT RYLTRNL (SEQ ID NO: 375) |
| | | wherein X₁ is V or A, X₂ is S, G, V or A, X₃ is R or H, X₄ is T, S, |
| | | V, Y, A or P and X₅ is S or A |
| AAV5- | RHVSASD | MSFVDHPPDW LEEVGEGLRE FLGLEAGPPK PKPNQQHQDQ ARGLVLPGYN YLGPGNGLDR |
| peptide | (SEQ ID | GEPVNRADEV AREHDISYNE QLEAGDNPYL KYNHADAEFQ EKLADDTSFG GNLGKAVFQA |
| 113 | NO: 113) | KKRVLEPFGL VEEGAKTAPT GKRIDDHFPK RKKARTEEDS KPSTSSDAEA GPSGSQQLQI |
| | | PAQPASSLGA DTMSAGGGGP LGDNNQGADG VGNASGDWHC DSTWMGDRVV TKSTRTWVLP |
| | | SYNNHQYREI KSGSVDGSNA NAYFGYSTPW GYFDFNRFHS HWSPRDWQRL INNYWGFRPR |
| | | SLRVKIFNIQ VKEVTVQDST TTIANNLTST VQVFTDDDYQ LPYVVGNGTE GCLPAFPPQV |
| | | FTLPQYGYAT LNRDNTENPT ERSSFFCLEY FPSKMLRTGN NFEFTYNFEE VPFHSSFAPS |
| | | QNLFKLANPL VDQYLYRFVS TNNTGGVQFN KNLAGRYANT YKNWFPGPMG RTCGWNLGSG |
| | | VNRASVSAFA TTNRMELEGA SYQVPPQPNG MTNNLQGSNT YALENTMIFN SQPANPGTTA |
| | | TYLEGNMLIT SESETQPVNR VAYNVGGQMA TNNQSSTTRHVSASD AP ATGTYNLQEI |
| | | VPGSVWMERD VYLQGPIWAK IPETGAHFHP SPAMGGFGLK HPPPMMLIKN TPVPGNITSF |
| | | SDVPVSSFIT QYSTGQVTVE MEWELKKENS KRWNPEIQYT NNYNDPQFVD FAPDSTGEYR |
| | | TTRPIGTRYL TRPL (SEQ ID NO: 376) |
| AAV5- | VRSDRD | MSFVDHPPDW LEEVGEGLRE FLGLEAGPPK PKPNQQHQDQ ARGLVLPGYN YLGPGNGLDR |
| peptide | Q (SEQ | GEPVNRADEV AREHDISYNE QLEAGDNPYL KYNHADAEFQ EKLADDTSFG GNLGKAVFQA |
| 114 | ID NO: | KKRVLEPFGL VEEGAKTAPT GKRIDDHFPK RKKARTEEDS KPSTSSDAEA GPSGSQQLQI |
| | 114) | PAQPASSLGA DTMSAGGGGP LGDNNQGADG VGNASGDWHC DSTWMGDRVV TKSTRTWVLP |
| | | SYNNHQYREI KSGSVDGSNA NAYFGYSTPW GYFDFNRFHS HWSPRDWQRL INNYWGFRPR |
| | | SLRVKIFNIQ VKEVTVQDST TTIANNLTST VQVFTDDDYQ LPYVVGNGTE GCLPAFPPQV |
| | | FTLPQYGYAT LNRDNTENPT ERSSFFCLEY FPSKMLRTGN NFEFTYNFEE VPFHSSFAPS |
| | | QNLFKLANPL VDQYLYRFVS TNNTGGVQFN KNLAGRYANT YKNWFPGPMG RT@GWNSGSG |
| | | VNRASVSAFA TTNRMELEGA SYQVPPQPNG MTNNLQGSNT YALENTMIFN SQPANPGTTA |
| | | TYLEGNMLIT SESETQPVNR VAYNVGGQMA TNNQSSTTVRSDRDQ AP ATGTYNLQEI |
| | | VPGSVWMERD VYLQGPIWAK IPETGAHFHP SPAMGGFGLK HPPPMMLIKN TPVPGNITSF |
| | | SDVPVSSFIT QYSTGQVTVE MEWELKKENS KRWNPEIQYT NNYNDPQFVD FAPDSTGEYR |
| | | TTRPIGTRYL TRPL (SEQ ID NO: 377) |
| AAV5- | TVVTSIN | MSFVDHPPDW LEEVGEGLRE FLGLEAGPPK PKPNQQHQDQ ARGLVLPGYN YLGPGNGLDR |
| peptide | (SEQ ID | GEPVNRADEV AREHDISYNE QLEAGDNPYL KYNHADAEFQ EKLADDTSFG GNICKAVFQA |
| 115 | NO: 115) | KKRVLEPFGL VEEGAKTAPT GKRIDDHFPK RKKARTEEDS KPSTSSDAEA GPSGSQQLQI |
| | | PAQPASSLGA DTMSAGGGGP LGDNNQGADG VGNASGDWHC DSTWMGDRVV TKSTRTWVLP |
| | | SYNNHQYREI KSGSVDGSNA NAYFGYSTPW GYFDFNRFHS HWSPRDWQRL INNYWGFRPR |
| | | SLRVKIFNIQ VKEVTVQDST TTIANNLTST VQVFTDDDYQ LPYVVGNGTE GCLPAFPPQV |
| | | FTLPQYGYAT LNRDNTENPT ERSSFFCLEY FPSKMLRTGN NFEFTYNFEE VPFHSSFAPS |
| | | QNLFKLANPL VDQYLYRFVS TNNTGGVQFN KNLAGRYANT YKNWFPGPMG RT@GWNSGSG |
| | | VNRASVSAFA TTNRMELEGA SYQVPPQPNG MTNNLQGSNT YALENTMIFN SQPANPGTTA |
| | | TYLEGNMLIT SESETQPVNR VAYNVGGQMA TNNQSSTTTVVTSIN AP ATGTYNLQEI |
| | | VPGSVWMERD VYLQGPIWAK IPETGAHFHP SPAMGGFGLK HPPPMMLIKN TPVPGNITSF |
| | | SDVPVSSFIT QYSTGQVTVE MEWELKKENS KRWNPEIQYT NNYNDPQFVD FAPDSTGEYR |
| | | TTRPIGTRYL TRPL (SEQ ID NO: 378) |

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A recombinant adeno-associated virus (rAAV) capsid protein comprising a peptide insertion of at least 4 and up to 7 contiguous amino acids, said peptide insertion being immediately after an amino acid residue corresponding to one of amino acids 451 to 461 of AAV9 capsid protein of FIG. 1, wherein said peptide insertion has an amino acid sequence of one of SEQ ID NOs: 1-138,
and wherein said capsid protein is a wild-type capsid protein or a variant capsid protein having up to three amino acid substitutions, and
wherein an rAAV vector prepared from the capsid protein comprising the peptide insertion has enhanced tropism to a target tissue compared to an rAAV vector prepared from a capsid protein without the peptide insertion or a reference capsid protein.

2. The rAAV capsid protein of claim 1, wherein said capsid protein is from at least one AAV type selected from AAV type 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype hu.31 (AAVhu.31), serotype hu.32 (AAVhu.32), serotype rh10 (AAVrh10), serotype rh20 (AAVrh20), serotype hu.37 (AVVhu.37), serotype rh39 (AAVrh39), and serotype rh74 (AAVrh74), or said variant capsid protein is AAV9.AAA or AAVhu.32.AAA or a capsid protein that has 90%, 95%, or 99% sequence identity thereto.

3. The rAAV capsid protein of claim 1 or 2, wherein said peptide insertion occurs immediately after one of the amino acid residues within:
(a) 450-459 of AAV1 capsid amino acid sequence (SEQ ID NO: 139);
(b) 449-458 of AAV2 capsid amino acid sequence (SEQ ID NO: 140);
(c) 449-459 of AAV3 capsid amino acid sequence (SEQ ID NO: 141);
(d) 443-453 of AAV4 capsid amino acid sequence (SEQ ID NO: 142);
(e) 442-445 of AAV5 capsid amino acid sequence (SEQ ID NO: 143);
(f) 450-459 of AAV6 capsid amino acid sequence (SEQ ID NO: 144);
(g) 451-461 of AAV7 capsid amino acid sequence (SEQ ID NO: 145);
(h) 451-461 of AAV8 capsid amino acid sequence (SEQ ID NO: 146);
(i) 451-461 of AAV9 capsid amino acid sequence (SEQ ID NO: 151);
(j) 451-461 of AAVhu.32 capsid amino acid sequence (SEQ ID NO: 148);
(k) 452-461 of AAVrh10 capsid amino acid sequence (SEQ ID NO: 152);
(l) 452-461 of AAVrh20 capsid amino acid sequence (SEQ ID NO: 155);
(m) 452-461 of AAVhu.37 capsid amino acid sequence (SEQ ID NO: 153);
(n) 452-461 of AAVrh39 capsid amino acid sequence (SEQ ID NO: 150);
(o) 452-461 of AAVrh74 capsid amino acid sequence (SEQ ID NO: 156 or SEQ ID NO: 157); or
(p) 452-461 of AAV9.AAA capsid amino acid sequence (SEQ ID NO: 158) as depicted in FIG. 1.

4. The rAAV capsid protein of claim 3, wherein said peptide insertion occurs after an amino acid residue corresponding to one of amino acids 1451, N452, G453, S454, G455, Q456, N457, Q458, Q459, T460, or L461 of the AAV9 capsid or variant AAV9.AAA capsid.

5. The rAAV capsid protein of claim 4, wherein said peptide insertion occurs after an amino acid residue corresponding to amino acid S454 of the AAV9 capsid protein, the AAV.32 capsid protein, variant AAV9.AAA capsid protein, or variant AAVhu.32.AAA capsid protein.

6. The rAAV capsid protein of any one of claims 1-5 wherein the peptide is 7 amino acids.

7. The rAAV capsid protein of any one of claims 1-6 wherein the reference capsid protein is AAV9, AAV9.AAA, AAVhu32, or AAVhu32.AAA.

8. The rAAV capsid protein of any one of claims 1 through 7, wherein said peptide insertion comprises an amino acid sequence of at least 4 and up to 7 contiguous amino acids of SEQ ID NO: 135, SEQ ID NO: 136, SEQ ID NO: 137 or SEQ ID NO: 138, where SEQ ID NO: 135 is X₁-Q-V-X₂-X₃-X₄-X₅, wherein X₁ is V or A, X₂ is S, G, V or A, X₃ is R or H, X₄ is any amino acid and X₅ is S, G, V or A, where SEQ ID NO: 136 is X₁-Q-V-X₂-X₃-X₄-X₅, wherein X₁ is V or A, X₂ is S, G, V or A, X₃ is R or H, X₄ is any amino acid and X₅ is S or A, where SEQ ID NO: 137 is X₁-Q-V-X₂-X₃-X₄-X₅, wherein X₁ is V or A, X₂ is S, G, V or A, X₃ is R or H, X₄ is T, S, V, Y, A or P and X₅ is S, G, V or A, or where SEQ ID NO: 138 is X₁-Q-V-X₂-X₃-X₄-X₅, wherein X₁ is V or A, X₂ is S, G, V or A, X₃ is R or H, X₄ is T, S, V, Y, A or P and X₅ is S or A.

9. The rAAV capsid protein of claim 8, wherein the peptide insertion has an amino acid sequence of VQVGRAA (SEQ ID NO: 5), VQVGRTS (SEQ ID NO: 8), AQVGRAS (SEQ ID NO: 24), VQVGRVS (SEQ ID NO: 36), VQVGRSS (SEQ ID NO: 44), VQVGRYS (SEQ ID NO: 130), VQVGRAS (SEQ ID NO: 131), VQVGRPS (SEQ ID NO: 132), VQVVRPS (SEQ ID NO: 133) or VQVGHAS (SEQ ID NO: 134).

10. The rAAV capsid protein of claim 8 or 9 which has an amino acid sequence of SEQ ID No. 263, 264, 265 or 266.

11. The rAAV capsid protein of any one of claims 1-7, wherein said peptide insertion comprises an amino acid sequence of at least 4 and up to 7 contiguous amino acids of SGTVIRS (SEQ ID NO: 1), TRQYVPG (SEQ ID NO: 4), VQVGRTS (SEQ ID NO: 8), RGAVQKV (SEQ ID NO: 13), GQVHQAR (SEQ ID NO: 32), TSGGQIR (SEQ ID NO: 38), HMGHSGK (SEQ ID NO: 88), MRAVSQL (SEQ ID NO: 109), PRQYVPG (SEQ ID NO: 110), RSSSGR (SEQ ID NO: 111), VVKSTKS (SEQ ID NO: 112), RHVSASD (SEQ ID NO: 113), VRSDRDQ (SEQ ID NO: 114), or TVVTSIN (SEQ ID NO: 115).

12. The rAAV capsid protein of claim 11 wherein said peptide insertion is SGTVIRS (SEQ ID NO: 1), TRQYVPG (SEQ ID NO: 4), VQVGRTS (SEQ ID NO: 8), RGAVQKV (SEQ ID NO: 13), GQVHQAR (SEQ ID NO: 32), TSGGQIR (SEQ ID NO: 38), HMGHSGK (SEQ ID NO: 88), MRAVSQL (SEQ ID NO: 109), PRQYVPG (SEQ ID NO: 110), RSSSGR (SEQ ID NO: 111), VVKSTKS (SEQ ID NO: 112), RHVSASD (SEQ ID NO: 113), VRSDRDQ (SEQ ID NO: 114), or TVVTSIN (SEQ ID NO: 115).

13. The rAAV capsid protein of any one of claims 1 through 7, wherein said peptide insertion comprises an amino acid sequence of at least 4 and up to 7 contiguous amino acids of AQVGRAS (SEQ ID NO: 24), SVTSVRV (SEQ ID NO: 37), SIAKNSA (SEQ ID NO: 76), DGRRIGV (SEQ ID NO: 116), TSGERRG (SEQ ID NO: 117), PSSVQHR (SEQ ID NO: 118), SSSVQHR (SEQ ID NO: 119), SGMQERR (SEQ ID NO: 120), LERGNLE (SEQ ID NO: 121), YRDVRQT (SEQ ID NO: 122), PSAVQHR (SEQ ID NO: 123), YVGGRAV (SEQ ID NO: 124), IGSRGVA (SEQ ID NO: 125), SDVSRPR (SEQ ID NO: 126), GSVRQAA (SEQ ID NO: 127), QSPHTSQ (SEQ ID NO: 128), or ASQAYHG (SEQ ID NO: 128).

14. The rAAV capsid protein of claim 13, wherein said peptide insertion is AQVGRAS (SEQ ID NO: 24), SVTSVRV (SEQ ID NO: 37), SIAKNSA (SEQ ID NO: 76), DGRRIGV (SEQ ID NO: 116), TSGERRG (SEQ ID NO: 117), PSSVQHR (SEQ ID NO: 118), SSSVQHR (SEQ ID NO: 119), SGMQERR (SEQ ID NO: 120), LERGNLE (SEQ ID NO: 121), YRDVRQT (SEQ ID NO: 122), PSAVQHR (SEQ ID NO: 123), YVGGRAV (SEQ ID NO: 124), IGSRGVA (SEQ ID NO: 125), SDVSRPR (SEQ ID NO: 126), GSVRQAA (SEQ ID NO: 127), QSPHTSQ (SEQ ID NO: 128), or ASQAYHG (SEQ ID NO: 128).

15. The rAAV capsid protein of claim 14, wherein said peptide insertion is AQVGRAS (SEQ ID NO: 24), PSSVQHR (SEQ ID NO: 118), YVGGRAV (SEQ ID NO: 124), IGSRGVA (SEQ ID NO: 125) or GSVRQAA (SEQ ID NO: 127).

16. The rAAV capsid protein of claim 14, wherein said peptide insertion is DGRRIGV (SEQ ID NO: 116), SSSVQHR (SEQ ID NO: 119), YRDVRQT (SEQ ID NO: 122) or PSAVQHR (SEQ ID NO: 123).

17. The rAAV capsid protein of claim 1, having the amino acid sequence of any one of the amino acid sequences of SEQ ID NO: 159-266 and 268-375.

18. The rAAV capsid protein of any one of the preceding claims, wherein said target tissue with enhanced tropism is skeletal muscle or heart muscle.

19. The rAAV capsid protein of claim 18, wherein an rAAV vector prepared from the capsid protein exhibits an at least 2 fold, 5 fold, 10 fold, 15 fold, 20 fold, or 25 fold greater transduction of skeletal muscle and/or heart muscle than an rAAV vector prepared from the capsid protein without the peptide insert or the reference capsid protein.

20. The rAAV capsid protein of any one of the preceding claims wherein the target tissue is CNS neurons.

21. The rAAV capsid protein of claim 20 wherein an rAAV vector prepared from the capsid protein has an at least 2 fold, 5 fold, 10 fold, 15 fold, 20 fold, or 25 fold greater transduction of CNS neurons than an rAAV vector prepared from the capsid protein without the peptide insert or the reference capsid protein.

22. The rAAV capsid protein of any one of the preceding claims wherein an rAAV vector prepared from the capsid protein has reduced transduction of liver, heart or astrocytes than an rAAV vector prepared from the capsid protein without the peptide insert or the reference capsid protein.

23. The rAAV capsid protein of claim 22 wherein the rAAV vector prepared from the capsid protein has an at least 2 fold, 5 fold, 10 fold, 15 fold, 20 fold, 25 fold or 40 fold lower transduction of liver than an rAAV vector prepared from the capsid protein without the peptide insert or the reference capsid protein.

24. The rAAV capsid protein of any one of claims 19, 21, 22, or 23 wherein the reference capsid protein is AAV9 capsid or AAVhu.32 capsid.

25. A nucleic acid comprising a nucleotide sequence encoding the rAAV capsid protein of any one of the preceding claims, or encoding an amino acid sequence sharing at least 80% identity therewith.

26. The nucleic acid of claim 25 which encodes the rAAV capsid protein of any one of the preceding claims.

27. A packaging cell capable of expressing the nucleic acid of claim 25 or 26 to produce AAV vectors comprising the capsid protein encoded by said nucleotide sequence.

28. A rAAV vector comprising the capsid protein of any one of claims 1-24.

29. The rAAV vector of claim 28, further comprising a rAAV genome comprising a transgene flanked by AAV ITR sequences.

30. A pharmaceutical composition comprising the rAAV vector of claim 28 or 29 and a pharmaceutically acceptable carrier.

31. A method of delivering a transgene to a cell, said method comprising contacting said cell with the rAAV vector of claim 28 or 29; or the rAAV vector of claim 28 or 29 for use in delivering a transgene to a cell, wherein said cell is contacted with the vector.

32. A method of delivering a transgene to a target tissue of a subject in need thereof, said method comprising administering to said subject the rAAV vector of claim 28 or 29; or the rAAV vector of claim 28 or 29 for use in delivering a transgene to a target tissue of a subject in need thereof, wherein the vector is administered to said subject.

33. The method, or rAAV vector for use, according to claim 32, wherein said rAAV vector is administered systemically, intravenously, intrathecally, intra-nasally, intra-peritoneally, intravitreally, via lumbar puncture or via the cisterna magna.

34. The method, or rAAV vector for use, according to claim 32 or claim 33, wherein said target tissue is muscle, retina or CNS.

35. A method of making a recombinant AAV (rAAV) vector library comprising
(i) producing a starting plasmid have a gene expression cassette comprising a nucleic acid sequence encoding an AAV capsid protein, wherein a stop codon is placed at a target insertion site within the nucleic acid sequence encoding the AAV capsid protein;
(ii) providing a population of nucleic acids which encode a repertoire of peptides to produce a peptide library;
(iii) creating individual plasmids based on the starting plasmid 1) each comprising the nucleic acid encoding the AAV capsid with a nucleic acid encoding a random peptide from the peptide library inserted at the target insertion site of the nucleic acid encoding the capsid protein thus replacing the stop codon, and 2) each plasmid further containing a nucleic acid encoding a barcode for identification of the nucleic acid encoding the capsid and peptide insert placed before the 5′- or after the 3′-end of the nucleic acid encoding the capsid;
(iv) collecting the individual plasmids;
(v) transfecting a population of cells with the collection of individual plasmids and one or more plasmids carrying an rAAV genome comprising a transgene and necessary genes;
(vi) culturing the population of transfected cells under appropriate conditions to produce a collection of rAAV vectors each comprising the AAV capsid having the peptide insert encapsidating the rAAV genome comprising the transgene; and
(vii) harvesting the rAAV vector library.

36. The method of claim 35, wherein the capsid gene encodes an AAV1 capsid protein (SEQ ID NO: 139), an AAV4 capsid protein (SEQ ID NO: 142), an AAV5 capsid protein (SEQ ID NO: 143), an AAV8 capsid protein (SEQ ID NO: 146), an AAV9 capsid protein (SEQ ID NO: 151), an AAV9.AAA capsid protein (SEQ ID No: 158), AAVhu.32 (SEQ ID NO: 148), AAVhu.32.AAA (SEQ ID NO. 267), an AAV3B capsid protein (SEQ ID NO: 154) or an AAVhu37 capsid protein (SEQ ID NO: 153).

37. The method of any one of claims 34 to 36, wherein the target insertion site is at or after an amino acid residue within VR-4 or VR-8 of the capsid protein

38. The method of claim 37, wherein after the target insertion site is after an amino acid corresponding to S454 of AAV9 (SEQ ID NO: 151).

39. The method of any one of claims 34 to 38 wherein the peptide is a 7 amino acid peptide.

40. An rAAV vector library made by the method of any one of claims 34 to 39.

41. An rAAV vector library of claim 40 wherein at least 80%, 85%, 90%, 95%, 98%, 99% or 100% of the rAAV vectors in the library have a capsid with a peptide insert.


SEQUENCE LISTING (378 sequences)

SEQ ID NO: 1 - protein, 7 aa, synthetic construct
SGTVIRS

SEQ ID NO: 2 - protein, 5 aa, synthetic construct
GRPAP

SEQ ID NO: 3 - protein, 7 aa, synthetic construct
KDQKIGP

SEQ ID NO: 4 - protein, 7 aa, synthetic construct
TRQYVPG

SEQ ID NO: 5 - protein, 7 aa, synthetic construct
VQVGRAA

SEQ ID NO: 6 - protein, 7 aa, synthetic construct
FTQSRGG

SEQ ID NO: 7 - protein, 7 aa, synthetic construct
SRSDRPA

SEQ ID NO: 8 - protein, 7 aa, synthetic construct
VQVGRTS

SEQ ID NO: 9 - protein, 7 aa, synthetic construct
SSGSQKF

SEQ ID NO: 10 - protein, 7 aa, synthetic construct
SGTVLRS

SEQ ID NO: 11 - protein, 7 aa, synthetic construct
SHTKSAT

SEQ ID NO: 12 - protein, 7 aa, synthetic construct
DQARVRI

SEQ ID NO: 13 - protein, 7 aa, synthetic construct
RGAVQKV

SEQ ID NO: 14 - protein, 7 aa, synthetic construct
VGRDSRS

SEQ ID NO: 15 - protein, 7 aa, synthetic construct
SRSDRQF

SEQ ID NO: 16 - protein, 7 aa, synthetic construct
VTRSQIM

SEQ ID NO: 17 - protein, 7 aa, synthetic construct
QVGARHP

SEQ ID NO: 18 - protein, 7 aa, synthetic construct
GRAAASL

SEQ ID NO: 19 - protein, 7 aa, synthetic construct
PSGVPIR

SEQ ID NO: 20 - protein, 7 aa, synthetic construct
MSYNQAT

SEQ ID NO: 21 - protein, 7 aa, synthetic construct
ITGGVRV

SEQ ID NO: 22 - protein, 7 aa, synthetic construct
SRSDRQV

SEQ ID NO: 23 - protein, 7 aa, synthetic construct
TSGVPIR

SEQ ID NO: 24 - protein, 7 aa, synthetic construct
AQVGRAS

SEQ ID NO: 25 - protein, 7 aa, synthetic construct
VKASHHS

SEQ ID NO: 26 - protein, 7 aa, synthetic construct
SHTKTVT

SEQ ID NO: 27 - protein, 7 aa, synthetic construct
GSVQTVR

SEQ ID NO: 28 - protein, 7 aa, synthetic construct
DTRIRSM

SEQ ID NO: 29 - protein, 7 aa, synthetic construct
SRTKTAT

SEQ ID NO: 30 - protein, 7 aa, synthetic construct
QVGARQS

SEQ ID NO: 31 - protein, 7 aa, synthetic construct
PSGQRLH

SEQ ID NO: 32 - protein, 7 aa, synthetic construct
GQVHQAR

SEQ ID NO: 33 - protein, 7 aa, synthetic construct
TSGVQIR

SEQ ID NO: 34 - protein, 7 aa, synthetic construct
SIAKTSS

SEQ ID NO: 35 - protein, 7 aa, synthetic construct
SRADRHV

SEQ ID NO: 36 - protein, 7 aa, synthetic construct
VQVGRVS

SEQ ID NO: 37 - protein, 7 aa, synthetic construct
SVTSVRV

SEQ ID NO: 38 - protein, 7 aa, synthetic construct
TSGGQIR

SEQ ID NO: 39 - protein, 7 aa, synthetic construct
SHIKTAT

SEQ ID NO: 40 - protein, 7 aa, synthetic construct
SHIKTST

SEQ ID NO: 41 - protein, 7 aa, synthetic construct
GNIKYGS

SEQ ID NO: 42 - protein, 7 aa, synthetic construct
SRSDRHV

SEQ ID NO: 43 - protein, 7 aa, synthetic construct
TSGAPIR

SEQ ID NO: 44 - protein, 7 aa, synthetic construct
VQVGRSS

SEQ ID NO: 45 - protein, 7 aa, synthetic construct
MKYRGTD

SEQ ID NO: 46 - protein, 7 aa, synthetic construct
HFAGKSV

SEQ ID NO: 47 - protein, 7 aa, synthetic construct
IKYRGTD

SEQ ID NO: 48 - protein, 7 aa, synthetic construct
SIAKHSS

SEQ ID NO: 49 - protein, 7 aa, synthetic construct
SHTRTAT

SEQ ID NO: 50 - protein, 7 aa, synthetic construct
SGTGIRA

SEQ ID NO: 51 - protein, 7 aa, synthetic construct
SIAKHGA

SEQ ID NO: 52 - protein, 7 aa, synthetic construct
GRAAATL

SEQ ID NO: 53 - protein, 7 aa, synthetic construct
SGTVLRA

SEQ ID NO: 54 - protein, 7 aa, synthetic construct
ASAKHAA

SEQ ID NO: 55 - protein, 7 aa, synthetic construct
AHTKTAT

SEQ ID NO: 56 - protein, 7 aa, synthetic construct
VKASHAS

SEQ ID NO: 57 - protein, 7 aa, synthetic construct
SNTKHAT

SEQ ID NO: 58 - protein, 7 aa, synthetic construct
VPVGRAS

SEQ ID NO: 59 - protein, 7 aa, synthetic construct
SISKHSA

SEQ ID NO: 60 - protein, 7 aa, synthetic construct
QKGRNPD

SEQ ID NO: 61 - protein, 7 aa, synthetic construct
QSAVRHP

SEQ ID NO: 62 - protein, 7 aa, synthetic construct
VQGGRAS

SEQ ID NO: 63 - protein, 8 aa, synthetic construct
VKNSSGTF

SEQ ID NO: 64 - protein, 7 aa, synthetic construct
HFTHQSK

SEQ ID NO: 65 - protein, 7 aa, synthetic construct
DKRGVIV

SEQ ID NO: 66 - protein, 7 aa, synthetic construct
HMGQSGK

SEQ ID NO: 67 - protein, 7 aa, synthetic construct
GQVHQSR

SEQ ID NO: 68 - protein, 7 aa, synthetic construct
SHTKPAT

SEQ ID NO: 69 - protein, 7 aa, synthetic construct
KDSGGYR

SEQ ID NO: 70 - protein, 7 aa, synthetic construct
RRVSNVD

SEQ ID NO: 71 - protein, 7 aa, synthetic construct
ARVTASI

SEQ ID NO: 72 - protein, 7 aa, synthetic construct
GNIKSGS

SEQ ID NO: 73 - protein, 7 aa, synthetic construct
DKRGVIG

SEQ ID NO: 74 - protein, 7 aa, synthetic construct
SIAKTAA

SEQ ID NO: 75 - protein, 7 aa, synthetic construct
QSVGRYS

SEQ ID NO: 76 - protein, 7 aa, synthetic construct
SIAKNSA

SEQ ID NO: 77 - protein, 8 aa, synthetic construct
VSGAVRVT

SEQ ID NO: 78 - protein, 7 aa, synthetic construct
PSSKARQ

SEQ ID NO: 79 - protein, 7 aa, synthetic construct
MARPQSI

SEQ ID NO: 80 - protein, 7 aa, synthetic construct
TVRGREQ

SEQ ID NO: 81 - protein, 7 aa, synthetic construct
SHTRTST

SEQ ID NO: 82 - protein, 7 aa, synthetic construct
HMGQYGK

SEQ ID NO: 83 - protein, 7 aa, synthetic construct
TVATSKI

SEQ ID NO: 84 - protein, 7 aa, synthetic construct
RAVTLGV

SEQ ID NO: 85 - protein, 7 aa, synthetic construct
FHTKTAT

SEQ ID NO: 86 - protein, 5 aa, synthetic construct
QQAQR

SEQ ID NO: 87 - protein, 7 aa, synthetic construct
SIARHSA

SEQ ID NO: 88 - protein, 7 aa, synthetic construct
HMGHSGK

SEQ ID NO: 89 - protein, 7 aa, synthetic construct
ATRGVIG

SEQ ID NO: 90 - protein, 7 aa, synthetic construct
SKTFAAS

SEQ ID NO: 91 - protein, 7 aa, synthetic construct
VVAGAMR

SEQ ID NO: 92 - protein, 7 aa, synthetic construct
SSGAQKF

SEQ ID NO: 93 - protein, 7 aa, synthetic construct
EMTSRHV

SEQ ID NO: 94 - protein, 7 aa, synthetic construct
RRVSTVD

SEQ ID NO: 95 - protein, 7 aa, synthetic construct
ITGEVRG

SEQ ID NO: 96 - protein, 7 aa, synthetic construct
IQGVHSK

SEQ ID NO: 97 - protein, 5 aa, synthetic construct
SPARA

SEQ ID NO: 98 - protein, 5 aa, synthetic construct
AGEGP

SEQ ID NO: 99 - protein, 5 aa, synthetic construct
AGPGA

SEQ ID NO: 100 - protein, 5 aa, synthetic construct
RQARR

SEQ ID NO: 101 - protein, 7 aa, synthetic construct
GNIQSRS

SEQ ID NO: 102 - protein, 7 aa, synthetic construct
IIHNQAK

SEQ ID NO: 103 - protein, 7 aa, synthetic construct
PKNVKTT

SEQ ID NO: 104 - protein, 7 aa, synthetic construct
DMNSRHV

SEQ ID NO: 105 - protein, 7 aa, synthetic construct
ENRGIIG

SEQ ID NO: 106 - protein, 7 aa, synthetic construct
YTKTSSV

SEQ ID NO: 107 - protein, 7 aa, synthetic construct
LVNQQKS

SEQ ID NO: 108 - protein, 7 aa, synthetic construct
PSSKDRH

SEQ ID NO: 109 - protein, 7 aa, synthetic construct
MRAVSQL

SEQ ID NO: 110 - protein, 7 aa, synthetic construct
PRQYVPG

SEQ ID NO: 111 - protein, 6 aa, synthetic construct
RSSSGR

SEQ ID NO: 112 - protein, 7 aa, synthetic construct
VVKSTKS

SEQ ID NO: 113 - protein, 7 aa, synthetic construct
RHVSASD

SEQ ID NO: 114 - protein, 7 aa, synthetic construct
VRSDRDQ

SEQ ID NO: 115 - protein, 7 aa, synthetic construct
TVVTSIN

SEQ ID NO: 116 - protein, 7 aa, synthetic construct
DGRRIGV

SEQ ID NO: 117 - protein, 7 aa, synthetic construct
TSGERRG

SEQ ID NO: 118 - protein, 7 aa, synthetic construct
PSSVQHR

SEQ ID NO: 119 - protein, 7 aa, synthetic construct
SSSVQHR

SEQ ID NO: 120 - protein, 7 aa, synthetic construct
SGMQERR

SEQ ID NO: 121 - protein, 7 aa, synthetic construct
LERGNLE

SEQ ID NO: 122 - protein, 7 aa, synthetic construct
YRDVRQT

SEQ ID NO: 123 - protein, 7 aa, synthetic construct
PSAVQHR

SEQ ID NO: 124 - protein, 7 aa, synthetic construct
YVGGRAV

SEQ ID NO: 125 - protein, 7 aa, synthetic construct
IGSRGVA

SEQ ID NO: 126 - protein, 7 aa, synthetic construct
SDVSRPR

SEQ ID NO: 127 - protein, 7 aa, synthetic construct
GSVRQAA

SEQ ID NO: 128 - protein, 7 aa, synthetic construct
QSPHTSQ

SEQ ID NO: 129 - protein, 7 aa, synthetic construct
ASQAYHG

SEQ ID NO: 130 - protein, 7 aa, synthetic construct
VQVGRYS

SEQ ID NO: 131 - protein, 7 aa, synthetic construct
VQVGRAS

SEQ ID NO: 132 - protein, 7 aa, synthetic construct
VQVGRPS

SEQ ID NO: 133 - protein, 7 aa, synthetic construct
VQVVRPS

SEQ ID NO: 134 - protein, 7 aa, synthetic construct
VQVGHAS

SEQ ID NO: 135-138 - skipped (4 entries)

SEQ ID NO: 139 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 140 - protein, 733 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTR

SEQ ID NO: 141 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 142 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 143 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 144 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 145 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 146 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 147 - protein, 736 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 148 - protein, 737 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 149 - protein, 13 aa, synthetic construct
AAAAAAAAAAAAA

SEQ ID NO: 150 - protein, 13 aa, synthetic construct
AAAAAAAAAAAAA

SEQ ID NO: 151 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 152 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 153 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 154 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 155 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 156 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 157 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 158 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 159 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVIRSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 160 - protein, 741 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRPAPGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 161 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSKDQKIGPGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 162 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTRQYVPGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 163 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRAAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 164 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSFTQSRGGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 165 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRPAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 166 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRTSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 167 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSGSQKFGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 168 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVLRSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 169 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKSATGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 170 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDQARVRIGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 171 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRGAVQKVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 172 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVGRDSRSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 173 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQFGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 174 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVTRSQIMGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 175 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARHPGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 176 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRAAASLGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 177 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSGVPIRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 178 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMSYNQATGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 179 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSITGGVRVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 180 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 181 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTSGVPIRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 182 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSAQVGRASGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 183 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVKASHHSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 184 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKTVTGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 185 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVQTVRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 186 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDTRIRSMGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 187 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRTKTATGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 188 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARQSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 189 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSVTSVRVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 190 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMKYRGTDGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 191 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSIAKNSAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 192 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHMGQYGKGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 193 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMRAVSQLGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 194 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPRQYVPGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 195 - protein, 742 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRSSSGRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 196 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVVKSTKSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 197 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSSVQHRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 198 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSSVQHRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 199 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGMQERRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 200 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLERGNLEGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 201 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYRDQRVTGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 202 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSAVQHRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 203 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYVGGRAVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 204 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSIGSRGVAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 205 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSDVSRPRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 206 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVRQAAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 207 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQSPHTSQGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 208 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASQAYHGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 209 - protein, 743 aa, synthetic construct
  VARIANT 455: note = X is V or A
  VARIANT 458: note = X is S, G, V or A
  VARIANT 459: note = X is R or H
  VARIANT 460: note = X is any amino acid
  VARIANT 461: note = X is S, G, V or A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSXQVXXXXGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 210 - protein, 743 aa, synthetic construct; identical to SEQ ID NO: 209
  VARIANT 455: note = X is V or A
  VARIANT 458: note = X is S, G, V or A
  VARIANT 459: note = X is R or H
  VARIANT 460: note = X is any amino acid
  VARIANT 461: note = X is S or A

SEQ ID NO: 211 - protein, 743 aa, synthetic construct; identical to SEQ ID NO: 209
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S, G, V or A

SEQ ID NO: 212 - protein, 743 aa, synthetic construct; identical to SEQ ID NO: 209
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S or A

SEQ ID NO: 213 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVIRSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 214 - protein, 741 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRPAPGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 215 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSKDQKIGPGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 216 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTRQYVPGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 217 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRAAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 218 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSFTQSRGGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 219 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRPAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 220 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRTSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 221 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSGSQKFGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 222 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVLRSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 223 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKSATGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 224 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDQARVRIGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 225 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRGAVQKVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 226 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVGRDSRSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 227 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQFGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 228 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVTRSQIMGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 229 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARHPGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 230 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRAAASLGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 231 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSGVPIRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 232 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMSYNQATGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 233 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSITGGVRVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 234 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 235 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTSGVPIRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 236 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSAQVGRASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 237 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVKASHHSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 238 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKTVTGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 239 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVQTVRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 240 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDTRIRSMGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 241 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRTKTATGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 242 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARQSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 243 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSVTSVRVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 244 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMKYRGTDGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 245 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSIAKNSAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 246 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHMGQYGKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 247 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMRAVSQLGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 248 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPRQYVPGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 249 - protein, 742 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRSSSGRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 250 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVVKSTKSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 251 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSSVQHRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 252 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSSVQHRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 253 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGMQERRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 254 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLERGNLEGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 255 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYRDQRVTGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 256 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSAVQHRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 257 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYVGGRAVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 258 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSIGSRGVAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 259 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSDVSRPRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 260 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVRQAAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 261 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQSPHTSQGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 262 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASQAYHGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 263 - protein, 743 aa, synthetic construct
  VARIANT 455: note = X is V or A
  VARIANT 458: note = X is S, G, V or A
  VARIANT 459: note = X is R or H
  VARIANT 460: note = X is any amino acid
  VARIANT 461: note = X is S, G, V or A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSXQVXXXXGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 264 - protein, 743 aa, synthetic construct; identical to SEQ ID NO: 263
  VARIANT 455: note = X is V or A
  VARIANT 458: note = X is S, G, V or A
  VARIANT 459: note = X is R or H
  VARIANT 460: note = X is any amino acid
  VARIANT 461: note = X is S or A

SEQ ID NO: 265 - protein, 743 aa, synthetic construct; identical to SEQ ID NO: 263
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S, G, V or A

SEQ ID NO: 266 - protein, 743 aa, synthetic construct; identical to SEQ ID NO: 263
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S or A

SEQ ID NO: 267 - protein, 737 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 268 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVIRSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 269 - protein, 742 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRPAPGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 270 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSKDQKIGPGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 271 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTRQYVPGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 272 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRAAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 273 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSFTQSRGGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 274 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRPAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 275 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRTSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 276 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSGSQKFGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 277 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVLRSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 278 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKSATGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 279 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDQARVRIGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 280 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRGAVQKVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 281 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVGRDSRSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 282 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQFGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 283 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVTRSQIMGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 284 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARHPGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 285 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRAAASLGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 286 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSGVPIRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 287 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMSYNQATGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 288 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSITGGVRVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 289 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 290 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTSGVPIRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 291 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSAQVGRASGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 292 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVKASHHSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 293 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKTVTGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 294 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVQTVRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 295 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDTRIRSMGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 296 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRTKTATGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 297 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARQSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 298 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSVTSVRVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 299 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMKYRGTDGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 300 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSIAKNSAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 301 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHMGQYGKGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 302 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMRAVSQLGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 303 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPRQYVPGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 304 - protein, 743 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRSSSGRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 305 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVVKSTKSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 306 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSSVQHRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 307 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSSVQHRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 308 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGMQERRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 309 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLERGNLEGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 310 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYRDVRQTGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 311 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSAVQHRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 312 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYVGGRAVGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 313 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSIGSRGVAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 314 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSDVSRPRGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 315 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVRQAAGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 316 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQSPHTSQGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 317 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASQAYHGGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 318 - protein, 744 aa, synthetic construct
  VARIANT 455: note = X is V or A
  VARIANT 458: note = X is S, G, V or A
  VARIANT 459: note = X is R or H
  VARIANT 460: note = X is any amino acid
  VARIANT 461: note = X is S, G, V or A
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSXQVXXXXGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 319 - protein, 744 aa, synthetic construct; identical to SEQ ID NO: 318
  VARIANT 455: note = X is V or A
  VARIANT 458: note = X is S, G, V or A
  VARIANT 459: note = X is R or H
  VARIANT 460: note = X is any amino acid
  VARIANT 461: note = X is S or A

SEQ ID NO: 320 - protein, 751 aa, synthetic construct
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S, G, V or A
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSXQVXXXXGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLSEQIDNO

SEQ ID NO: 321 - protein, 744 aa, synthetic construct; identical to SEQ ID NO: 318
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S or A

SEQ ID NO: 322 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVIRSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 323 - protein, 742 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRPAPGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 324 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSKDQKIGPGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 325 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTRQYVPGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 326 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRAAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 327 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSFTQSRGGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 328 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRPAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 329 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVQVGRTSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 330 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSGSQKFGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 331 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGTVLRSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 332 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKSATGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 333 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDQARVRIGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 334 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRGAVQKVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 335 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVGRDSRSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 336 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQFGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 337 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVTRSQIMGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 338 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARHPGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 339 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGRAAASLGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 340 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSGVPIRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 341 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMSYNQATGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 342 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSITGGVRVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 343 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRSDRQVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 344 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTSGVPIRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 345 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSAQVGRASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 346 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVKASHHSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 347 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSHTKTVTGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 348 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVQTVRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 349 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDTRIRSMGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 350 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSRTKTATGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 351 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQVGARQSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 352 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSVTSVRVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 353 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMKYRGTDGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 354 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSIAKNSAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 355 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHMGQYGKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 356 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSMRAVSQLGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 357 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPRQYVPGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 358 - protein, 743 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRSSSGRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 359 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSVVKSTKSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 360 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSSVQHRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 361 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSSSVQHRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 362 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSGMQERRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 363 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLERGNLEGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 364 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYRDVRQTGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 365 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPSAVQHRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 366 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSYVGGRAVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 367 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSIGSRGVAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 368 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSDVSRPRGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 369 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGSVRQAAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 370 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSQSPHTSQGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 371 - protein, 744 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASQAYHGGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 372 - protein, 744 aa, synthetic construct
  VARIANT 455: note = X is V or A
  VARIANT 458: note = X is S, G, V or A
  VARIANT 459: note = X is R or H
  VARIANT 460: note = X is any amino acid
  VARIANT 461: note = X is S, G, V or A
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSXQVXXXXGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 373 - protein, 744 aa, synthetic construct; identical to SEQ ID NO: 372
  VARIANT 455: note = X is V or A
  VARIANT 458: note = X is S, G, V or A
  VARIANT 459: note = X is R or H
  VARIANT 460: note = X is any amino acid
  VARIANT 461: note = X is S or A

SEQ ID NO: 374 - protein, 744 aa, synthetic construct; identical to SEQ ID NO: 372
  VARIANT 455: note = X is V or A
  VARIANT 458: note = X is S, G, V or A
  VARIANT 459: note = X is R or H
  VARIANT 460: note = X is T, S, V, Y, A or P
  VARIANT 461: note = X is S, G, V or A

SEQ ID NO: 375 - protein, 744 aa, synthetic construct; identical to SEQ ID NO: 372
  VARIANT 455: note = X is V or A
  VARIANT 458: note = X is S, G, V or A
  VARIANT 459: note = X is R or H
  VARIANT 460: note = X is T, S, V, Y, A, or P
  VARIANT 461: note = X is S or A

SEQ ID NO: 376 - protein, 731 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTRHVSASDAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 377 - protein, 731 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTVRSDRDQAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 378 - protein, 731 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTTVVTSINAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL